(19) United States
(12) Patent Application Publication
(10) Pub. No.: US 2002/0045175 A1
(43) Pub. Date: Apr. 18, 2002

(54) Gene recombination and hybrid protein development

(76) Inventors: Zhen-Gang Wang, Pasadena, CA (US); Christopher A. Voigt, Pasadena, CA (US); Stephen L. Mayo, Pasadena, CA (US); Frances H. Arnold, Pasadena, CA (US)

Correspondence Address: DARBY & DARBY, 805 THIRD AVENUE, 27TH FLR., NEW YORK, NY, 10022, US

(21) Appl. No.: 09/863,765

(22) Filed: May 23, 2001

Related U.S. Application Data
(60) Provisional application No. 60/207,048, filed on May 23, 2000.
(60) Provisional application No. 60/235,960, filed on Sep. 27, 2000.
(60) Provisional application No. 60/283,567, filed on Apr. 13, 2001.

(51) Int. Cl. C12Q 1/68; G01N 33/53; G06F 19/00; G01N 33/48; G01N 33/50

(52) U.S. Cl. 435/6; 435/7.1; 702/19; 702/20

(57) ABSTRACT

The invention relates to improved methods for directed evolution of polymers, including directed evolution of nucleic acids and proteins. Specifically, the methods of the invention include analytical methods for identifying “crossover locations” in a polymer. Crossovers at these locations are less likely to disrupt desirable properties of the protein, such as stability or functionality. The invention further provides improved methods for directed evolution wherein the polymer is selectively recombined at the identified “crossover locations”. Crossover disruption profiles can be used to identify preferred crossover locations. Structural domains of a biopolymer can also be identified and analyzed, and domains can be organized into schema. Schema disruption profiles can be calculated, for example based on conformational energy or interatomic distances, and these can be used to identify preferred or candidate crossover locations. Computer systems for implementing analytical methods of the invention are also provided.

This application claims priority under 35 U.S.C.§119(e) to co-pending U.S. Provisional Patent Application Ser. No. 60/207,048 (filed May 23, 2000), No. 60/235,960 (filed Sep. 27, 2000) and No. 60/283,567 (filed Apr. 13, 2001).

Numerous references, including patents, patent applications and various publications are cited and discussed in this specification. The citation and/or discussion of such references is provided to clarify the description of the invention and is not an admission that any such reference is “prior art” to the invention described herein. All references cited and discussed in this specification are incorporated by reference in their entirety and to the same extent as if each reference was individually incorporated by reference.

### 1. FIELD OF THE INVENTION

The invention relates to biomolecular engineering and design, including methods for the design and engineering of biopolymers such as proteins and nucleic acids.

More particularly, the invention relates to improved methods for in vivo and in vitro directed evolution of biopolymers, such as polypeptides (e.g. proteins) and oligonucleotides (e.g. DNA and RNA). The invention is particularly suited to techniques which generate hybrid biopolymers by recombining sequences of biopolymer building blocks, such as sequences of amino acid residues or nucleic acid residues, from more than one parent biopolymer (e.g. from two or more parent genes). This can be referred to as “crossing” two or more parents to produce recombinant offspring. Each location in the offspring where the biopolymer sequence changes or “crosses over” from one parent to another is called a “crossover location” or a “cut point.” A related term, known in the genetic algorithm literature, is “schema.” In the context of protein engineering, a schema is a representation or arrangement of polymer building blocks, such as nucleic or amino acid residues, or recognizable structural domains or energetic conformations, in which each building block contributes more or less to the structural integrity, form, function, or fitness of the polymer. In a recombination experiment, parents may have similar or different schema, and the offspring may preserve or disrupt, the schema of one or more parents. In a preferred embodiment of the invention, schema that are common to two or more parents are preserved in recombinant offspring.

The invention provides computational methods for predicting beneficial recombinations of biopolymers, e.g. the fragments, locations or schema of two or more parent genes which can advantageously be recombined. Directed evolution methods can be selected and applied to favor identified recombinations. By applying cut points at locations that preserve schema, the recombinant mutant library has a larger fraction of folded, stable hybrids or chimeras. Because the stability of the wild type is preserved, it is more likely that mutants exist in this library that have improvements in the desired properties.

For example, recombinant protocols can be modeled in silico to predict crossover locations which will tend to preserve and not disrupt, advantageous schema. The computational or in silico techniques of the invention can be used to determine preferred crossover locations. Residues of one or more biopolymers are identified (e.g. nucleotide residues of a nucleic acid or amino acid residues of a polypeptide) where crossover recombination may produce beneficial results, such as one or more improved properties. Preferably, improvements are obtained while minimally disrupting a desired biopolymer property, such as stability or functionality. Disruption is less likely when biopolymers are cut and recombined at structurally tolerant crossover sites determined according to the invention. Crossover locations on parent biopolymers are identified which tend to have little or no impact on the stability of the three-dimensional structure of the biopolymer, represented e.g., as schema, according to specified thresholds or parameters. These locations can be used as candidate crossover locations for recombination experiments. Alternatively, sets of interacting residues or schema can be identified which are collectively crucial or important to the structure of the biopolymer, according to specified threshold or parameters. Crossovers that disrupt these sets of beneficially interacting residues or schema are not desirable because they lead to destabilized structures, and thus can be ruled out.

These techniques provide a targeted approach for obtaining mutant or hybrid biopolymers with improved properties using directed evolution. For example, the invention is useful in the design of in vitro recombination experiments where nucleic acid sequences that encode two or more different parent proteins may be recombined to create hybrid sequences. Unlike other directed evolution methods, such as family shuffling, that require high sequence identity or similarity (e.g. 70% or higher), the invention can be applied to parent proteins of low sequence similarity, e.g. less than 50%, or of no sequence similarity (0%). For example, cut points for the recombination of proteins are selected based on preserving three-dimensional or conformational structure or structural motifs. Common structures or domains can be identified independently of amino acid sequence, or without requiring overall sequence similarity. Widely different sequences may code for the same or similar structures or schema. Different proteins with different functions may have similar structures. Such proteins can be identified and selected as parents for crossover recombination, at selected cut points which preserve or minimize disruption of common structures. This improves the likelihood of producing mutants with functions or properties from more than one parent. For example, a protease of high activity may be recombined, at selected cut points, with a second structurally similar protein of high thermal stability, to produce a thermostable protease with high activity. By focusing on structural similarity and by minimizing structural disruption, the invention provides mutants having new or improved properties, without needing to rely on serendipitous results from random recombinations of parents having a high sequence similarity. Recombination based on hybridization or sequence identity can be called “homologous” recombination. Recombination that is not based on sequence identity can be called “non-homologous” recombination. The invention encompasses both methods, which can be used independently, or together.

### 2. BACKGROUND OF THE INVENTION

The invention is concerned with polymers, primarily biopolymers such as polynucleotides (chains of nucleic acids, e.g. DNA and RNA) and polypeptides (chains of amino acids, e.g. proteins and enzymes). More particularly, the invention provides improved hybrid proteins and methods of obtaining them by crossover recombination.

Proteins are polypeptides that are useful to living organisms. For example, they provide structures in the body, do physical or chemical work, or act as catalysts for chemical reactions (i.e. as enzymes). Proteins are made by cells according to genetic information encoded, transcribed and translated by polynucleotides (DNA and RNA). It is often desirable to modify proteins so that they have new or improved properties. For example, a protein may be altered to increase its biological activity (e.g. its potency as an enzyme), or to improve its stability under different environmental conditions (e.g. temperature), or to change its function (e.g. to catalyze a different chemical reaction).

Nature makes these kinds of alterations in many ways, including for example genetic mutations, or changes due to the recombination of genetic material such as occurs from sexual reproduction. Changes that are beneficial tend to be preserved from generation to generation, while truly harmful changes may disappear over time, in a process called evolution. Changes which are neutral, i.e. neither helpful nor harmful, may also be preserved by default. This is a very long process, and tends to produce random changes which are then tested for survival by the environment. Scientists looking for proteins with improved properties have had the very difficult task of searching for changes in proteins at random, from the vast numbers of potential natural sources that are available. Changes that are desirable may not be produced or preserved by nature. Breeding experiments can be done to provide additional sources for genetic variation, tending toward traits of interest, but these techniques also are exceedingly slow, costly, and resource intensive. They are very inefficient, and may not produce desired results. For example, proteins that act as enzymes to break down other proteins can be used as stain-removing ingredients of a laundry detergent, but these proteins may have to work at higher temperatures than in nature.

Identifying proteins with desirable characteristics from nature, such as enzymes with improved heat resistance (thermal stability) or other fitness characteristics, has been a haphazard and difficult process. Accordingly, there has been a need for new ways to modify proteins, or the polynucleotides which encode them, to produce new proteins with improved properties or fitness. Two separate techniques commonly used to alter the properties of proteins and other biological molecules are directed evolution and computational design. The invention brings these techniques together, and in particular provides guided processes of genetic diversity that reduce the sequence space to be searched, are less prone to random results, and are more prone to produce proteins with improved fitness. According to the invention, preferred or optimal cut points for recombination, fragment sizes, and recombination strategies are provided. Structural information about parent proteins, such as knowledge of epitopes or active sites, or results of prior mutagenesis experiments, can be used to improve the outcome of protein evolution experiments. Other factors, such as library size and landscape data (e.g. structure/function relationships) can also be taken into account. Principles of statistical mechanics are applied to genetic algorithms, to produce computational models of evolutionary processes. These models correlate with observations and experiments in directed evolution, and can be adapted to different experimental designs. The computational models can also be used to provide a protein design model, which generates candidate recombinants in silico more rapidly than conventional in vitro methods, thus allowing experimental parameters to be rapidly tested and optimized.

Directed Evolution

Directed evolution techniques attempt to alter the properties of a biopolymer (e.g., a protein or a nucleic acid) by accumulating stepwise improvements through iterations of random mutagenesis, recombination and screening. See, e.g., Moore & Arnold, Nature Biotechnology 1996, 14:458; Miyazaki et al., J. Mol. Biol. 2000, 297:1015-1026; Arnold, Adv. Protein Chem. 2000, 55:ix-xi. Broadly speaking, these methods work by speeding up the natural processes of evolution. Changes in genetic material (e.g. mutations) are rapidly and artificially induced, typically in cells that can be easily and quickly grown in cell culture (e.g. outside the body). The resulting mutants are rapidly evaluated to identify new or improved properties or changes of interest.

In a typical in vitro protein evolution experiment, a naturally occurring or wild-type protein is identified, and its sequence is altered to produce diversity, for example by mutation or recombination. This results in large numbers of mutant proteins, which are screened according to appropriate fitness criteria, for example, the most active mutants that are reasonably stable may be selected. One or more of these mutants may then be selected as a parent for another round of evolution. This process may be repeated as desired, for example until no further improvements in fitness are observed.

Genetic recombination methods have been widely applied to accelerate in vitro protein evolution. Examples include DNA shuffling, random-priming recombination, and the staggered extensionprocess (StEP). See e.g., Stemmer, Proc. Natl. Acad. Sci., 91 :10747 (1994); Stemmer, Nature, 370:389 (1994); Zhao & Arnold, Nucleic Acids Res., 25:1307 (1997); Zhao et al., Nature Biotechnology, 49:290 (1998); Crameri et al.,, Nature, 391:288 (1998), Volkov et al., Methods Enzymol, 382:447-456 (2000).

Some of the advantages of directed evolution methods are that they can be used with large polymers, for example proteins with more than 500 amino acids; they produces unique and unexpected results; and polymers can be evolved to achieve several goals simultaneously. Some disadvantages are that directed evolution is limited by the genetic code. For example, there are sixty-four 3-base nucleic acid codons that code for 20 amino acids. A single mutation in a codon may not be enough for a wild-type amino acid to be changed into all 19 other possible amino acids. Often, two or more DNA mutations in the codon are required. In directed evolution experiments, the DNA mutation rate is small and the gene is large, so the probability of obtaining two neighboring DNA mutations is small. Practically, this means that not all amino acid mutations are possible using random mutagenesis alone. Nevertheless the number of hybrids which can be produced is vast, but even then they can not be made and screened as readily as would be desired. It is also difficult to produce simultaneous non-additive arrangements of sequences. A non-additive effect means that two or more simultaneous mutations have to be made in order to observe a fitness improvement. Often, the individual mutations lead to a decreased fitness. Because the mutation rate is small and the gene is large, there is a very small probability of obtaining the precise multiple-mutant needed to observe a non-additive change, and one that provides a benefit or fitness improvement.

Computational Design

Computational design, by contrast, has developed separately from directed evolution and is a fundamentally different approach. See, Street & Mayo, Structure 7:R105 (1999). Unlike the essentially random approach of directed evolution, computational design attempts to predict and then make the changes or mutations that will be beneficial or useful. Thus, the general objective of computational design is to identify particular interactions in a protein (or other biopolymer) that lead to desirable properties, and then modify the biopolymer sequence to optimize those interactions. For example, a force-field model can be used to quantitatively describe interactions between amino acid residues in a protein. An amino acid sequence may then be computed, at least in theory, to globally optimize these interactions. See e.g., Malakaukas & Mayo, Nature Structural Biology, 5:470 (1998); Dahiyat & Mayo, Science, 278:82 (1997).

Some of the advantages of computational protein design are that very large numbers of sequences can be screened in silico, e.g. 10²⁰⁻³⁰⁰; multiple mutations can be considered simultaneously; and all possible amino acid substitutions (the entire possible sequence space) can be searched. Some disadvantages are that computational requirements increase exponentially with larger polymer sequences; at least some structural information (e.g. a defined secondary sequence) is needed; and certain unique or unexpected possibilities may be overlooked because the polymer backbone is held constant for the calculations. In addition, it takes considerable if not restrictive computing power and computation time to calculate detailed energies between all possible amino acid combinations.

The Sequence Space

Computational design can effectively search a large sequence space, that is, a large number of sequences (e.g., >10²⁶). See, Dahiyat & Mayo, Science 278:82 (1997). However, the technique is currently limited by the size of the biopolymer. The largest full sequence design accomplished to date is a 28-mer zinc finger protein (id.). Partial designs can be done to improve the stability of proteins up to about 70 amino acids. Moreover, the technique currently is based on calculating the molecule's conformational energy, i.e. the relative energy of the molecule's folded and unfolded states. Thus, current computational methods have only been used to improve a molecule's stability. The technique has not been used to improve other properties of biopolymers, such as activity, selectivity, efficiency, or other characteristics of biological fitness.

Directed evolution methods, by contrast, have the benefit of improving any property in a molecule that can be detected and/or captured by a screen, for example catalytic activity of an enzyme. One effective and widely used directed evolution method involves production of a library of mutants from a parent sequence, e.g., by using error-prone PCR to produce random point mutations. Moore & Arnold, Nature Biotechnology, 14:458(1996); Miyazaki et al., J. Mol. Biol., 297:1015-1026 (2000). However, the technique is limited by several factors, one of which is the practical size of the screen. Zhao & Arnold, Curr. Op. St. Biol., 7, 480-485 (1997). Increasing the number of mutants screened enables the user to sample a larger fraction of possible sequences (a larger sequence space) and therefore provides better improvements in the properties of interest. However, the most mutants that may be observed in any practical screen or selection is between about 10³ to 10¹², depending upon the specific screening method. In comparison, however, an average protein of 300 residues will have at least 10³⁹⁰ possible amino acid combinations. Thus, any practical screening or selection assay can only search a small fraction of the possible sequences.

Moreover, the probability that any single random mutation will improve a property of the parent sequence is small, and the probability of improvement decreases rapidly when multiple simultaneous mutations are made. Furthermore, the negligible probability that two or three mutations occur in a single codon and the significant biases of error-prone PCR severely restrict the possible amino acid substitutions which may be searched. Again, there is a need to reduce the sequence space which must be searched in order to obtain desirable hybrids.

Family Shuffling (Recombination of Divergent Homologous Sequences)

Accumulating point mutations in a single sequence is an effective fine-tuning mechanism for directed evolution, but other methods can also be used to create molecular diversity, e.g. polymer sequences from which useful sequences can be identified by screening or selection. Mutations can be produced in vitro using error-prone PCR methods. Beneficial mutations can then be combined using genetic recombination methods. For example, a parent (e.g. wild-type) can be mutated to create a mutant library, which is then screened for desirable mutants. These mutants can then be used as parent genes in recombination experiments. The mutant parents are cut into fragments and the fragments are recombined to provide a library of recombinant mutants. The recombinant mutants can then be screened for beneficial or improved properties.

Recombination can be done without mutagenesis of a common parent. For example, two or more different but related parent genes can be recombined in a method known as “family shuffling” or “DNA shuffling.” Related sequences, e.g. from divergent homologous genes, can be cut and recombined to make hybrid genes. These methods generally rely on an assumption that the parent genes share closely related structures. See, e.g., Stemmer, Nature, 370:389 (1994); Volkov, A. A., et al., Methods Enzymol., 382:447-456 (2000); Crameri et al., Nature, 391:288 (1998). The shuffling process creates a library of many new genes which code for proteins with sequence information from any or all parents. For example, the first half of the sequence might come from one parent, while the second half might come from another. Another hybrid might have the first 20 nucleotides from one parent, the next 500 from another parent, and the last nucleotides from a third parent. The point at which a sequences derived from one parent switches to a sequence derived from another parent is called a crossover. There may be one or more crossovers in a given sequence.

A library of such hybrid genes might contain millions or trillions of different genes containing different patterns of crossovers. In family shuffling, genes from multiple parents and even from different species can be recombined, operations that do not occur in nature but which may nonetheless be useful for rapid adaptation. DNA shuffling is being used to generate improved proteins, and notably, proteins with features not present in one or all parent proteins, or not even known to occur in nature. See, Affholter & Arnold, “Engineering a revolution,” Chemistry in Britain, 35: 48-51 (1999); Ness et al., “Molecular Breeding—the natural approach to enzyme design,” Advances in Protein Chemistry, 55:261-292 (2000); Schmidt-Dannert, et al., “Molecular breeding of carotenoid biosynthetic pathways,” Nature Biotechnology, 18, 750-753 (2000).

DNA shuffling methods rely on hybridization between portions of the parent genes and can therefore only recombine closely related sequences, usually of more than 70% sequence identity. Furthermore, these methods generate crossovers between one parent sequence and another only in regions of the gene where there is high identity between the two sequences. Stated another way, recombination based on DNA sequence similarity requires overlap in the DNA between parents for a crossover to occur. The DNA of the parents is fragmented, and in order for the fragments to reanneal, they need to share some overlap to allow for DNA hybridization. The StEP protocol does not require as much overlap as the DNA shuffling protocol originally proposed by Stemmer. A variety of other shuffling techniques are also known, some of which do not require sequence identity or alignments. These include for example the ITCHY protocol. Ostermeier et al., Bioorganic & Medicinal Chem. 7:2139-2144 (1999); Ostermeier et al., Nature Biotechnol. 17:1205-1209 (1999).

Many proteins having similar three-dimensional structures show low or even no discemable sequence identity or similarity. Rational design (Mitra et al., Biochemistry, 32: 12959-12967 (1993); Shimoji et al., Biochemistry, 37: 8848-8852 (1998)), computational approaches (Bogarad & Deem, Proc. Natl. Acad. Sci. USA, 96:2591-95(1999)), and combinatorial methods (Ostermier et al., Nature Biotechnology, 17:1205-1209 (1999)) have shown that functional proteins can be obtained by recombination of such distantly related or low sequence similarity parent sequences. Accordingly there is a need for methods that can provide stable and functional hybrids from recombined parents having low or no sequence similarity or identity, but having three-dimensional structures in common.

Recombination can be performed using so-called “non-homologous” methods that do not need sequence identity or overlap, because the experimental protocol relies on other properties, and does not require DNA hybridization between the parents. Generally, two parents are recombined with a single crossover point using such methods. If recombination is restricted to a single crossover point between two parents, the crossover disruption of the recombinant mutants may be very substantially increased, leading to a library of less-stable mutants. According to the invention, non-homologous recombination protocols can be modeled or used together with improved and targeted computational methods to calculate crossover disruption profiles. These can be applied to favorably restrict crossover locations, minimize disruption, and select crossover regions and mutants that are more likely to be stable, and/or exhibit improved fitness.

Functional Crossover Locations

Random selection of crossover sites, as in conventional family shuffling, does not favor sites that are more likely to produce functional and improved mutants. Accordingly, methods of selecting promising crossover sites are needed. It has been empirically observed that functional shuffled sequences do not contain an even distribution of crossover locations throughout the sequence. For example, the crossover locations of some in vitro recombinant mutants are strongly biased towards the N- and C-termini of the resulting functional proteins. Ness et al., Nature Biotechnology, 1999, 17:893-896. Many of these crossovers at the termini do not, however, lead to functional improvements.

Sequence Databases

Given the explosive growth in the gene databases due to the exhaustive sequencing of large numbers of organisms, the sequences of homologous genes are easily accessible. However, to date, there is no rigorous method in the art to quantitatively use the information in sequence databases to identify optimal starting parents for recombination (e.g. shuffling) experiments. A method to rapidly and quantitatively use such information is desirable. It is further desirable to have methods that predict where crossover locations in recombination experiments are likely to generate functional proteins which also may have new and useful properties. Such methods would be useful for the creation of more diversity in a recombinant library, with a reduction in the numbers of mutants needed to be produced and screened. Methods that would address these and/or other problems in the art would allow the acceleration of in vitro protein evolution and would accelerate the creation of new proteins (e.g. enzymes) with novel and useful properties. This is of particular interest to those interested in improved protein-based drugs, and in the use of enzymes in industrial processes where enzymes must function in non-native environments or must catalyze non-native chemical reactions.

Thus, there is presently a need in the art for improved methods of designing biopolymers such as proteins and nucleic acids. Moreover, there exists a need for better methods for improving one or more properties of a biopolymer. There further exists a need for improved methods of directed evolution that overcome, at least partially, any one or more of the above-described problems in the art. For example, there is a need in the art to identify regions in the sequence of a molecule (e.g., a biopolymer such as a protein or nucleic acid) where crossover recombination is likely to generate a library of stable mutants or chimeras that can be screened for one or more beneficial and/or improved properties.

### 3. SUMMARY OF THE INVENTION

Applicants have discovered that producing mutant biopolymers by crossover recombination at certain cut point or locations is more likely to preserve stability and/or a desired property of the polymer, such as functionality, than crossovers in other areas. The crossover locations are identified by examining at what locations a crossover disrupts a schema structural domain or a minimum of coupling interactions between amino acid side chains of the polymer (e.g. polypeptide). The invention provides novel techniques for identifying residue locations where crossovers would disrupt a minimum of schema or coupling interactions in a polypeptide. These methods are straightforward and are computationally tractable.

Accordingly, a skilled artisan can readily use the methods to identify residues of a particular polymer sequence that permit crossover recombination with minimal disruption. The artisan may selectively recombine polymers at the identified crossover locations to generate recombinant mutants that are likely to be functional, and which can be screened for properties of interest. Such mutants are more likely to have one or more properties of interest that are improved over the properties of the parent polymer. Thus, by selectively recombining parent genes at identified crossover locations e.g. in silico, a skilled artisan may more readily and efficiently identify novel sequences with improved properties than if the artisan used randomized methods or conventional shuffling.

The invention therefore provides methods for selecting residues of a biopolymer sequence for crossover recombination by obtaining or determining which locations disrupt a structural domain or a minimal amount of coupling interactions in the amino acid sequence, and selecting the identified crossover locations. The polymers may be any type of polymer, including biopolymers such as, but not limited to, nucleic acids (comprising a sequence of nucleotide residues) and proteins or polypeptides (comprising a sequence of amino acid residues).

The invention also provides methods for the directed evolution of biopolymers. Two or more parent sequences are provided, each for example having one or more properties of interest, and one or more possible crossover locations. One or more recombinant polymers may then be generated from the parent polymer sequences, in which two or more of the parents are recombined at one or more selected crossover locations. These mutants are preferably screened for the one or more properties of interest. Mutants are selected where one or more properties of interest is modified and preferably is improved. In certain embodiments, the methods of the invention are iteratively repeated, and selected mutants are used as parent polymer sequences in subsequent iterations of the method.

The invention can also be used to identify optimal parent molecules (e.g. preferred parent genes) for recombination. Similar or structurally related parent molecules can be evaluated to determine which are more likely, when altered, to produce desirable improvements. For example, optimal parents can be mined from sequence databases, e.g. using disruption energy as a measure.

Computer systems are also provided that may be used to implement the analytical methods of the invention, including methods of identifying crossover locations in a polymer sequence and/or selecting such residues for mutation (e.g., as part of a directed evolution method). These computer systems comprise a processor interconnected with a memory that contains one or more software components. In particular, the one or more software components include programs that cause the processor to implement steps of the analytical methods described herein. The software components may further comprise additional programs and/or files including, for example, sequence or structural databases of polymers.

Computer program products are further provided, which comprise a computer readable medium, such as one or more floppy disks, compact discs (e.g., CD-ROMS or RW-CDS), DVDs, data tapes, etc., that have one or more software components encoded thereon in computer readable form. In particular, the software components may be loaded into the memory of a computer system and may then cause a processor of the computer system to execute steps of the analytical methods described herein. The software components may include additional programs and/or files including databases, e.g., of polymer sequences and/or structures.

### 4. BRIEF DESCRIPTION OF THE DRAWINGS

FIG. 1 is a flow diagram illustrating exemplary recombination embodiments of the methods of the invention.

FIG. 1A illustrates a method for determining a schema disruption profile.

FIG. 1B illustrates a method for modeling an experimental recombinant protocol.

FIG. 2 is a schematic illustration and graphical representation of crossover disruption.

FIG. 3 is a gene alignment for β-lactamase-like genes, (1) Enterobacter cloacae, (2) Citrobacter freundii, (3) Yersinia enterocolitica and (4) Klebsiella pneumonia. SWISPROT or TrEMBL accession numbers for the protein sequences and GenBank accession numbers for the DNA sequences are given.

FIG. 4A is an in silico probability distribution for all crossover locations calculated from a recombination algorithm for the four β-lactamase sequences of FIG. 3.

FIG. 4B is an in silico probability distribution of crossover locations for β-lactamase when screened for crossover locations that meet a set threshold. In this example, recombinant mutants are below the threshold E_{c}=14. The dark horizontal bars on the x-axis indicate the crossovers observed in prior in vitro experiment. Crameri et al., Nature, 391:288 (1998). These curves were calculated using Method 1 of the invention, described below. FIGS. 4C and 4D are similar to FIGS. 4A and 4B, but were calculated using Method 2 of the invention, described below.

FIG. 5 is a crossover disruption plot for non-homologous recombination experiments, using the ITCHY protocol, with glycinamide ribonucleotide transformylase. The sequence range 50-100, where recombinations were restricted in the experiments, is shown on the x-axis. The crossover disruption is shown on the y-axis.

FIG. 6 shows a probability distribution for schema disruption in computationally generated recombinant mutants. The probability distribution of the schema disruption is plotted for the recombinant mutants that contain at least three parents and is normalized by the total number of mutants. Each distribution represents the schema disruption of the portion of the recombinant mutants that contain each parent sequence: (1) Enterobacter cloacae, (2) Citrobacter freundii, (3) Yersinia enterocolitica, and (4) Klebsiella pneumoniae. The portion of the distribution that corresponds to the low-schema disruption is to the left of the black line (Schema Disruption, Sᵢ<18). In this region, the Klebsiella pneumonia (4) sequence corresponds with the least-disruptive schema. The addition of the Yersinia enterocolitica (3) sequence causes the most schema disruption, explaining why it was not observed in the functional hybrid proteins found in DNA shuffling experiments. The inset bar graph shows the integral between the schema disruption cutoff and zero. This represents the fraction of low-disruption schema associated with each parent.

FIG. 7 is an example of an in vitro method of overlap extension re-assembly, targeting identified crossover locations. The appropriate fragments may be obtained by split-pool synthesis.

FIG. 8A shows a fragment reassembly method using a parental template. The resulting products are subjected to heteroduplex recombination (Volkov et al., Nucl. Acids Res., 27:18 (1999)) to create libraries of genes within regions of non-identity. More complexity can be introduced by the addition of more fragments during template assembly.

FIG. 9 shows the preparation of gene fragments prepared by PCR with primers directed to regions targeted for crossovers.

FIG. 10 shows recombination directed to specific sites using crossover primers in DNA shuffling.

FIG. 11 shows an exemplary computer system that may be used to implement analytical methods of the invention.

FIG. 12 is a flow diagram illustrating one embodiment of a recombinant search algorithm of the invention, based on sequence identity.

FIG. 13 is a diagrammatic illustration of a computational algorithm used to generate recombinant mutants by DNA shuffling. (A) First, cut points are distributed randomly across the gene with probability p_{c}. In this diagram, the arrows mark cut points and the thatched line represent regions of sequence similarity between parents. (B) A parent is picked at random to determine the first fragment. The next fragment is chosen amongst the parents that share adequate sequence identity (including the parent of the previous fragment) with equal probability. (C) The complete library of recombinant mutants that can be generated by the cut pattern shown.

FIG. 14 is a flow chart of an exemplary algorithm for directed evolution experiments.

FIG. 15 shows a quantitative comparison of the energy (x-axis) and distance (y-axis) based calculations of crossover disruption for Transformylase. An energy cutoff of 0.2 kcal/mol and a distance cutoff of 4.0 angstroms were used. The data fits a linear correlation with R²=0.91.

FIG. 16 shows a comparison of crossover disruption calculations for Transformylase based on the distance (top) and energy (bottom) definitions of coupling. An energy cutoff of 0.2 kcal/mol and a distance cutoff of 4.0 angstroms were used. The qualitative shapes of both plots are similar.

FIG. 17 shows the crossover disruption of inserted phytase domains. The distance cut off d_{c} was set to 3.0 angstroms and the crossover disruption was normalized according to Equation (3). The experimental parameters are as reported by Lehmann and co-workers (2001).

FIG. 18 is a schematic of the hierarchal process of protein folding. First, the unfolded polypeptide rapidly collapses (“bursts”) into substructures. Next, the substructures condense to form the tertiary structure of the native protein. It is undesirable for crossovers to disrupt compact units that nucleate the remaining structure (“building blocks” or “schema”).

FIG. 19 is a schematic demonstrating the utility of a contact map in identifying compact units of substructure. A representative contact map is on the left. The graph on the right is a statistical study of the average length of contiguous residues that can fold into a sphere of the indicated diameter (Gilbert 1998). This information can be used in the following way. If a 15-residue segment can fold into a sphere with a diameter of 21 angstroms, then this segment could be considered as being of average compactness. However, if a 20-residue segment can fold into a sphere of 21 angstroms, this is considered as having a significantly above-average compactness. This is visualized on the contact map as a triangle on the diagonal formed by the cut points required to generate the segment. If the segment fits into a sphere of the specified diameter, then the triangle will be entirely white (interacting).

FIG. 20 is a comparison of (A) the Go-algorithm (using a diameter size dᵣₒₛₛ=21 angstroms) with (B) the 1 d crossover disruption profile oftransformylase. The Go-algorithm predicts that there are three domain-forming regions in the structure, whereas the 1d crossover disruption profile (threshold energy of 0.2 kcal/mol) demonstrates that one of these domain-forming regions is not sampled because it causes too much disruption.

FIG. 21 is a two-dimensional contact map of beta-lactamase using dᵣₒₛₛ=21. Black regions indicate resides that are further than 21 angstroms apart and white residues indicate residues that are closer than 21 angstroms. The lines indicate the approximate locations of crossovers observed experimentally by Crameri et al (1998).

FIG. 22 provides an analytical description of Go's algorithm for determining domains based on the contact map. The domain diameter dᵣₒₛₛ=21 for these calculations and Equation (8) is used to determine the domain-forming ability of each residue. Low regions in this graph indicate suitable places for domain boundaries. The thick black horizontal lines indicate the approximate domain boundaries identified by this method and the thin vertical lines demarcate the regions where crossovers were observed experimentally by Crameri et al (1998). The domain algorithm identifies some of the general structure of where the crossover occurs, but makes a poor prediction overall.

FIG. 23 shows an algorithm that combines the concept of disrupting a domain with the concept of disrupting coupling interactions. First, all fragments of size nₘᵢₙ and greater are identified in the structure. Next, the fragments that fold into a sphere of diameter dᵣₒₛₛ and are coupled to the remainder of the structure above a threshold disruption value are separated. Finally, the schema disruption value of all the residues involved in the interacting compact unit are incremented by one, indicating that crossovers that occur in this region will disrupt a “building block,” and therefore be destabilizing.

FIG. 24 shows the schema disruption profile as determined from the transformylase structure. (A) No sequence identity was considered (Pᵢ=Pⱼ=1 in Equation 3). The parameters are d_{c}=4. 0, E_{c,thesh}=4.0. (B) Sequence identity is considered (Equation 3). The parameters are d_{c}=4.0 and E_{c,thresh}=1.0. The normalization of crossover disruption in both graphs was according to Equation (6).

FIG. 25 shows the schema disruption profile as determined from the beta-lactamase structure compared with the experimentally observed crossover points (thick horizontal bars) (Crameri et al., 1998). (A) The profile as determined from the domain algorithm alone with dᵣₒₛₛ=21 angstroms and nₘᵢₙ=15 residues (Equation 9). (B) The profile with disruptive domains removed where the crossover disruption was normalized as in Equation (3). The crossover disruption threshold was set to be E_{c,thresh}=0.007 (corresponding to a Z-score of 0.1). No sequence identity was considered (Pᵢ=Pⱼ=1 in Equation 3). (C) The profile with disruptive domains removed where the crossover disruption was normalized as in Equation (6). The crossover disruption threshold was set to be E_{c,thresh}=4.0 (corresponding to a Z-score of 0.4). No sequence identity was considered (Pᵢ=Pⱼ=1 in Equation 3). (D) The same profile as in (C), except sequence identity is considered (Equation 3). The crossover disruption threshold was set to be E_{c,thresh}=0.6 (corresponding to a Z-score of 0.2).

FIG. 26A shows a schema disruption calculation of the P450 2C5 structure. Equation (10) was used to generate the graph and the crossover disruption normalization scheme of Equation (3) was used. The parameters for this calculation are d_{c}=4.0, E_{c,thresh}=0.005 (corresponding to a Z-score of 0.3). The red lines indicate where experimentally generated single cut point recombination events led to folded chimeras (Pikuleva et al, 1996). The arrow indicates the location of the crossover that resulted in a folded P450cam-P450 2C9 chimera (Shimoji et al, 1998). Note that not all of the residues were resolved in the structure, so the numbering starts at 30 (e.g., residue 1 in the graph is residue 30) and residues 212-222 are missing.

FIG. 26B shows a schema disruption calculation of the P450cam structure. Equation (10) was used to generate the graph and the crossover disruption normalization scheme of Equation (3) was used. The parameters for this calculation are d_{c}=4.0, E_{c, thresh}=0.007 (corresponding to a Z-score of 0.65). The red line indicates the location of the crossover that resulted in a folded P450cam-P450 2C9 chimera (Shimoji et al, 1998). Note that not all residues were resolved in the structure: residue 1 in the graph is residue 7 in the structure. No sequence identity was considered for either P450 5 calculation (Pᵢ=Pⱼ=1 in Equation 3).

FIGS. 27A and 27B illustrate a method for determining optimal parents for crossover recombination by analyzing the schema disruption experiment for a DNA shuffling experiment with beta-lactamase (Crameri et al., 1998). The parents in this example are: (1) Enterobacter cloacae, (2) Citrobacter freundii, (3) Yersinia enterocolitica, and (4) Klebsiella pneumoniae.

### 5. DETAILED DESCRIPTION OF THE INVENTION

The invention overcomes problems in the prior art and provides novel methods which can be used for directed evolution of biopolymers such as proteins and nucleic acids. 15 In particular, the invention provides methods which can be used to identify candidate locations in a biopolymer for crossovers, such that the biopolymer (e.g., polypeptide) will likely retain stability and functionality while allowing crossovers to occur. By generating hybrids that are recombined at selected candidate crossover locations or cut points, mutant or hybrid polymers having one or more improved properties may be more readily identified while simultaneously reducing the number(s) of mutants screened.

Details of the invention are described below, including specific examples. These examples are provided to illustrate embodiments of the invention. However, the invention is not limited to the particular embodiments, and many modifications and variations of the invention will be apparent to those skilled in the art. Such modifications and variations are also part of the invention.

5.1 Definitions

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and how to use them. The scope an meaning of any use of a term will be apparent from the specific context in which the term is used.

Molecular Biology

The term “molecule” means any distinct or distinguishable structural unit of matter comprising one or more atoms, and includes, for example, polypeptides and polynucleotides.

The term “polymer” means any substance or compound that is composed of two or more building blocks (‘mers’) that are repetitively linked together. For example, a “dimer” is a compound in which two building blocks have been joined togther; a “trimer” is a compound in which three building blocks have been joined together; etc.

A “biopolymer” is any polymer having an organic or biochemical utility or that is produced by a cell. Preferred biopolymers include, but are not limited to, polynucleotides, polypeptides and polysaccharides.

The term “polynucleotide” or “nucleic acid molecule” refers to a polymeric molecule having a backbone that supports bases capable of hydrogen bonding to typical polynucleotides, wherein the polymer backbone presents the bases in a manner to permit such hydrogen bonding in a specific fashion between the polymeric molecule and a typical polynucleotide (e.g., single-stranded DNA). Such bases are typically inosine, adenosine, guanosine, cytosine, uracil and thymidine. Polymeric molecules include “double stranded” and “single stranded” DNA and RNA, as well as backbone modifications thereof (for example, methylphosphonate linkages).

Thus, a “polynucleotide” or “nucleic acid” sequence is a series of nucleotide bases (also called “nucleotides”), generally in DNA and RNA, and means any chain of two or more nucleotides. A nucleotide sequence frequently carries genetic information, including the information used by cellular machinery to make proteins and enzymes. The terms include genomic DNA, cDNA, RNA, any synthetic and genetically manipulated polynucleotide, and both sense and antisense polynucleotides. This includes single- and double-stranded molecules; i.e., DNA-DNA, DNA-RNA, and RNA-RNA hybrids as well as “protein nucleic acids” (PNA) formed by conjugating bases to an amino acid backbone. This also includes nucleic acids containing modified bases, for example, thio-uracil, thio-guanine and fluoro-uracil.

The polynucleotides herein may be flanked by natural regulatory sequences, or may be associated with heterologous sequences, including promoters, enhancers, response elements, signal sequences, polyadenylation sequences, introns, 5′- and 3′-non-coding regions and the like. The nucleic acids may also be modified by many means known in the art. Non-limiting examples of such modifications include methylation, “caps”, substitution of one or more of the naturally occurring nucleotides with an analog, and intemucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.) and alkylators to name a few. The polynucleotides may be derivatized by formation of a methyl or ethyl phosphotriester or an alkyl phosphoramidite linkage. Furthermore, the polynucleotides herein may also be modified with a label capable of providing a detectable signal, either directly or indirectly. Exemplary labels include radioisotopes, fluorescent molecules, biotin and the like. Other non-limiting examples of modification which may be made are provided, below, in the description of the invention.

The term “oligonucleotide” refers to a nucleic acid, generally of at least 10, preferably at least 15, and more preferably at least 20 nucleotides, preferably no more than 100 nucleotides, that is hybridizable to a genomic DNA molecule, a cDNA molecule, or an mRNA molecule encoding a gene, mRNA, cDNA, or other nucleic acid of interest. Oligonucleotides can be labeled, e.g., with ³²P-nucleotides or nucleotides to which a label, such as biotin or a fluorescent dye (for example, Cy3 or Cy5) has been covalently conjugated. In one embodiment, an oligonucleotide can be used as PCR primers. Oligonucleotides therefore have many practical uses that are well known in the art. For example, a labeled oligonucleotide can be used as a probe to detect the presence of a nucleic acid. Generally, oligonucleotides are prepared synthetically, preferably on a nucleic acid synthesizer. Accordingly, oligonucleotides can be prepared with non-naturally occurring phosphoester analog bonds, such as thioester bonds, etc.

A “polypeptide” is a chain of chemical building blocks called amino acids that are linked together by chemical bonds called “peptide bonds”. The term “protein” refers to polypeptides that contain the amino acid residues encoded by a gene or by a nucleic acid molecule (e.g., an mRNA or a cDNA) transcribed from that gene either directly or indirectly. Optionally, a protein may lack certain amino acid residues that are encoded by a gene or by an mRNA. For example, a gene or mRNA molecule may encode a sequence of amino acid residues on the N-terminus of a protein (i.e., a signal sequence) that is cleaved from, and therefore may not be part of, the final protein. A protein or polypeptide, including an enzyme, may be a “native” or “wild-type”, meaning that it occurs in nature; or it may be a “mutant”, “variant” or “modified”, meaning that it has been made, altered, derived, or is in some way different or changed from a native protein or from another mutant.

“Amplification” of a polynucleotide denotes the use of polymerase chain reaction (PCR) to increase the concentration of a particular DNA sequence within a mixture of DNA sequences. For a description of PCR see Saiki et al., Science 1988, 239:487.

A “gene” is a sequence of nucleotides which code for a functional “gene product”. Generally, a gene product is a functional protein. However, a gene product can also be another type of molecule in a cell, such as an RNA (e.g., a tRNA or a rRNA). For the purposes of the invention, a gene product also refers to an mRNA sequence which may be found in a cell. For example, measuring gene expression levels according to the invention may correspond to measuring mRNA levels. A gene may also comprise regulatory (i.e., non-coding) sequences as well as coding sequences. Exemplary regulatory sequences include promoter sequences, which determine, for example, the conditions under which the gene is expressed. The transcribed region of the gene may also include untranslated regions including introns, a 5′-untranslated region (5′-UTR) and a 3′-untranslated region (3′-UTR).

A “coding sequence” or a sequence “encoding” an expression product, such as a RNA, polypeptide, protein or enzyme, is a nucleotide sequence that, when expressed, results in the production of that RNA, polypeptide, protein or enzyme; i.e., the nucleotide sequence “encodes” that RNA or it encodes the amino acid sequence for that polypeptide, protein or enzyme.

A “promoter sequence” is a DNA regulatory region capable of binding RNA polymerase in a cell and initiating transcription of a downstream (3′ direction) coding sequence. A promoter sequence is typically bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently found, for example, by mapping with nuclease S1), as well as protein binding domains (consensus sequences) responsible for the binding of RNA polymerase.

A coding sequence is “under the control of” or is “operatively associated with” transcriptional and translational control sequences in a cell when RNA polymerase transcribes the coding sequence into RNA, which is then trans-RNA spliced (if it contains introns) and, if the sequence encodes a protein, is translated into that protein.

The term “express” and “expression” means allowing or causing the information in a gene or DNA sequence to become manifest, for example producing RNA (such as rRNA or mRNA) or a protein by activating the cellular functions involved in transcription and translation of a corresponding gene or DNA sequence. A DNA sequence is expressed by a cell to form an “expression product” such as an RNA (e.g., a mRNA or a rRNA) or a protein. The expression product itself, e.g., the resulting RNA or protein, may also said to be “expressed” by the cell.

The term “transfection” means the introduction of a foreign nucleic acid into a cell. The term “transformation” means the introduction of a “foreign” (i.e., extrinsic or extracellular) gene, DNA or RNA sequence into a host cell so that the host cell will express the introduced gene or sequence to produce a desired substance, in this invention typically an RNA coded by the introduced gene or sequence, but also a protein or an enzyme coded by the introduced gene or sequence. The introduced gene or sequence may also be called a “cloned” or “foreign” gene or sequence, may include regulatory or control sequences (e.g., start, stop, promoter, signal, secretion or other sequences used by a cell's genetic machinery). The gene or sequence may include nonfunctional sequences or sequences with no known function. A host cell that receives and expresses introduced DNA or RNA has been “transformed” and is a “transformant” or a “clone”. The DNA or RNA introduced to a host cell can come from any source, including cells of the same genus or species as the host cell or cells of a different genus or species.

The terms “vector”, “cloning vector” and “expression vector” mean the vehicle by which a DNA or RNA sequence (e.g., a foreign gene) can be introduced into a host cell so as to transform the host and promote expression (e.g., transcription and translation) of the introduced sequence. Vectors may include plasmids, phages, viruses, etc. and are discussed in greater detail below.

A “cassette” refers to a DNA coding sequence or segment of DNA that codes for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct.” A common type of vector is a “plasmid”, which generally is a self-contained molecule of double-stranded DNA, usually of bacterial origin, that can readily accept additional (foreign) DNA and which can readily introduced into a suitable host cell. A large number of vectors, including plasmid and fungal vectors, have been described for replication and/or expression in a variety of eukaryotic and prokaryotic hosts.

The term “host cell” means any cell of any organism that is selected, modified, transformed, grown or used or manipulated in any way for the production of a substance by the cell. For example, a host cell may be one that is manipulated to express a particular gene, a DNA or RNA sequence, a protein or an enzyme. Host cells can further be used for screening or other assays that are described infra. Host cells may be cultured in vitro or one or more cells in a non-human animal (e.g., a transgenic animal or a transiently transfected animal).

The term “expression system” means a host cell and compatible vector under suitable conditions, e.g. for the expression of a protein coded for by foreign DNA carried by the vector and introduced to the host cell. Common expression systems include E. coli host cells and plasmid vectors, insect host cells such as Sf9, Hi5 or S2 cells and Baculovirus vectors, Drosophila cells (Schneider cells) and expression systems, fish cells and expression systems (including, for example, RTH-149 cells from rainbow trout, which are available from the American Type Culture Collection and have been assigned the accession no. CRL-1710) and mammalian host cells and vectors.

The terms “mutant” and “mutation” mean any change in a particular polymer sequence (also sometimes referred to herein as a “parent sequence”). Mutations may include, but are not limited to, changes in the nucleotide sequence of a nucleic acid (including changes in the sequence of a gene), and also changes in the amino acid sequence of a protein or polypeptide. Thus, in the invention these terms may refer to a difference of even one residue (e.g. one nucleic or amino acid), but more typically refer to recombined sequences that are substantially different from their parents. That is, a “mutant” includes the offspring of recombined parent sequences, as by combining (for example) genetic material from two parent genes. A mutant may also be referred to as a “hybrid” or a “variant.”

The term “chimera” is synonymous with “recombinant mutant” and refers to an offspring gene which contains genetic material from one or more parents.

The methods of the invention may include steps of comparing parent sequences to each other or a parent sequence to one or more mutants. Such comparisons typically comprise alignments of polymer sequences, e.g., using sequence alignment programs and/or algorithms that are well known in the art (for example, BLAST, FASTA and MEGALIGN, to name a few). The skilled artisan can readily appreciate that, in such alignments, where a mutation contains a residue insertion or deletion, the sequence alignment will introduce a “gap” (typically represented by a dash, “-”, or “Δ”) in the polymer sequence not containing the inserted or deleted residue. Thus, for example, in an embodiment where a mutation introduces a single amino acid deletion in a parent sequence at amino acid residue i, an alignment of the parent and mutant polypeptide sequences will introduce a gap in the mutant sequence that aligns with amino acid residue i of the parent. In such embodiments, therefore, amino acid residue i in the mutant sequence is preferably said to be a “gap” or “deletion”.

The term “heterologous” refers to a combination of elements not naturally occurring. For example, chimeric RNA molecules may comprise an rRNA sequence and a heterologous RNA sequence which is not part of the rRNA sequence. In this context, the heterologous RNA sequence refers to an RNA sequence that is not naturally located within the ribosomal RNA sequence. Alternatively, the heterologous RNA sequence may be naturally located within the ribosomal RNA sequence, but is found at a location in the rRNA sequence where it does not naturally occur. As another example, heterologous DNA refers to DNA that is not naturally located in the cell, or in a chromosomal site of the cell. Preferably, heterologous DNA includes a gene foreign to the cell. A heterologous expression regulatory element is a regulatory element operatively associated with a different gene than the one it is operatively associated with in nature.

The term “homologous” refers to the relationship between two biopolymers (e.g. polypeptides or oligonucleotides) that possess a common evolutionary origin. This includes, without limitation, proteins from superfamilies (e.g., the immunoglobulin superfamily) in the same species of organism, as well as homologous proteins from different species of organism (for example, myosin light chain polypeptide, etc.; see, Reeck et al., Cell 1987, 50:667). Such proteins (and their encoding nucleic acids) have sequence homology, as reflected by their sequence similarity, or regions of sequence similarity, however expressed. For example, “homology” can be expressed as sequence similarity in terms of percent sequence identity or by the presence of specific residues or motifs and conserved positions.

The terms “sequence similarity” and “sequence identity”, in all their grammatical forms, refers to the degree of identity or correspondence between nucleic acid or amino acid sequences that may or may not share a common evolutionary origin (see, Reeck et al., supra). However, in common usage and in the instant application, the term “homologous”, particularly when modified with an adverb such as “highly”, may refer to sequence similarity and may or may not relate to a common evolutionary origin.

The term “recombination” and variant spellings thereof, encompasses both “homologous” and “non-homologous” recombination. In its most basic form, recombination is the exchange of biopolymer fragments between two biopolymer sequences. As defined in this invention, sequences may be recombined at the amino acid or nucleic acid level.

The term “homologous recombination” refers to the exchange of biopolymer fragments between two or more biopolymer sequences at locations where the sequences exhibit regions of sequence homology. In more general biological terms, recombination refers to the insertion of a modified or foreign DNA sequence contained by a first vector into another DNA sequence contained in second vector, or a chromosome of a cell. The first vector targets a specific chromosomal site for homologous recombination. For homologous recombination, the first vector will contain sufficiently long region of homology to sequences of the second vector or chromosome to allow complementary binding and incorporation of DNA from the first vector into the DNA of the second vector, or the chromosome.

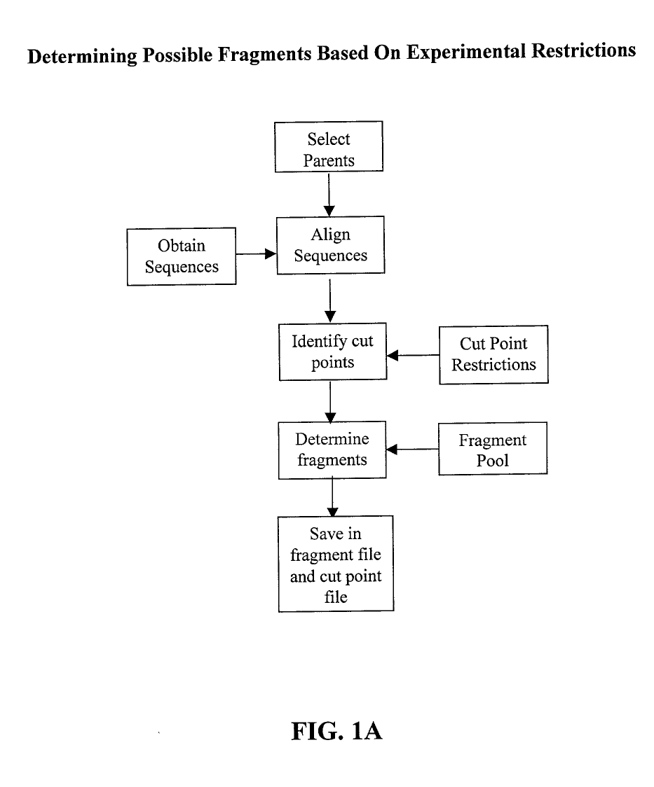

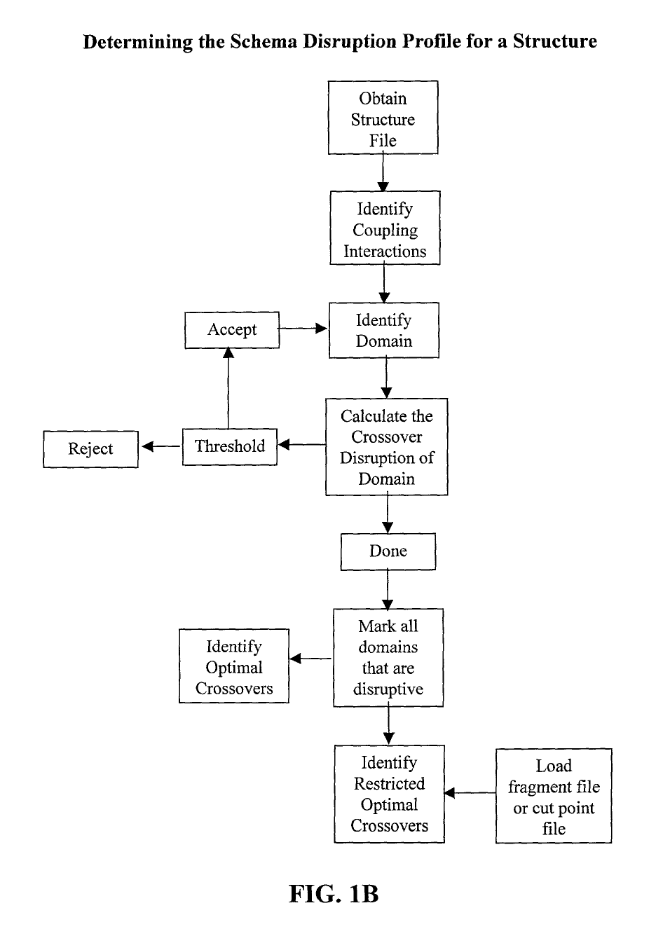

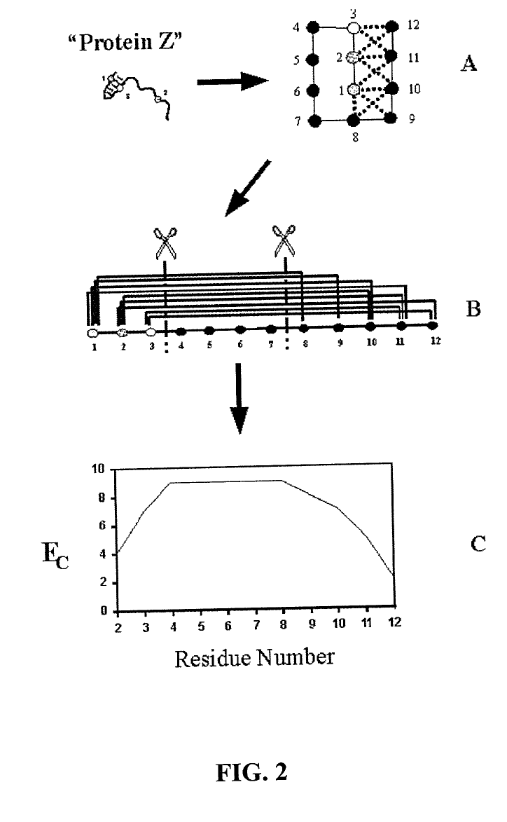

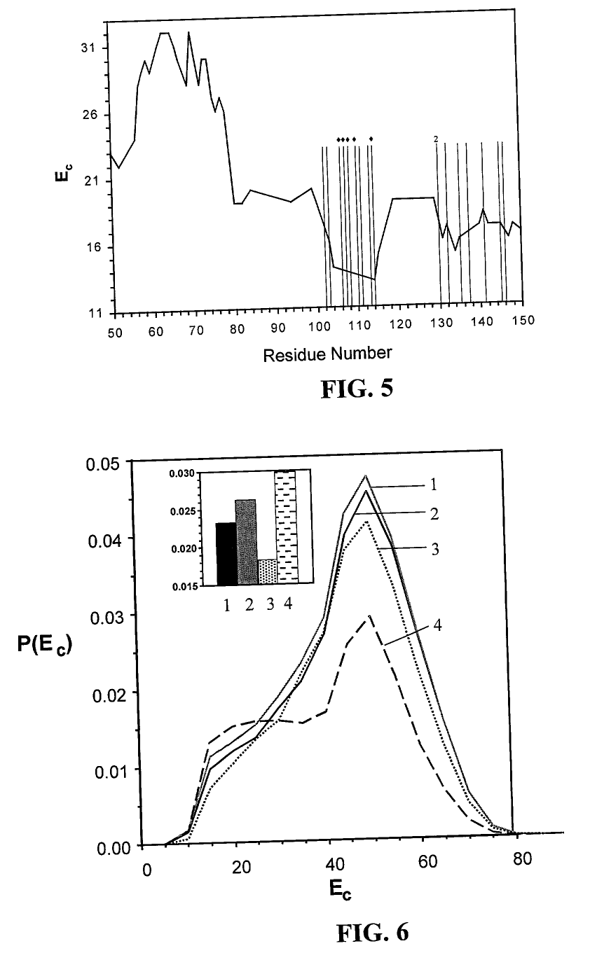

According to the invention, the sequence similarity of biopolymers being recombined can be high, low, or none, and indeed can range from less than 50% (e.g., 0% to as high as 100%. Where parent sequences are homologous, i.e. have some threshold of sequence identity, alignments may be used to aid in the selection of cut points and fragments for recombination. Alignments are also used for certain recombination protocols, such as DNA shuffling, which can be modeled according to the invention. However, other recombinations do not require alignments, such as the ITCHY protocol, and these also can be modeled to calculate a schema disruption profile. A model of non-homologous (non-sequence identity) recombination is illustrated by FIG. 1A and FIG. 5, discussed infra. Crossovers can be calculated for 0% sequence identity, as long as the parents fold into the same (or similar) structures. Cut points are determined as in FIG. 2, which does not require or imply sequence identity.

The term “non-homologous recombination” refers to the exchange of biopolymer fragments between two biopolymer sequences that are not homologous, or that do not share sequence identity, for example according to a given threshold. As used herein, non- homologous biopolymers, like homologous biopolymers, may or may not have a common evolutionary origin, and in preferred embodiments they do have a common evolutionary origin. However, non-homologous biopolymers, unlike homologous biopolymers, have no sequence identity, or the sequence identity (if any) is less than a given minimum.

In certain embodiments of the invention, biopolymers or fragments thereof may be selected for recombination based on any suitable energy or structural data, not necessarily homology or sequence identity. For example, cut points or schema may be selected based on structural input such as interatomic distances, without regard for sequence identity. That is, the biopolymers may or may not have any, or a given degree, of sequence identity. Optimal schema (and fragments) can be determined from this data without regard for the recombination or shuffling protocol. In addition, alignment data from homologous sequences or regions, if any, can be used as additional structural input to further refine the selected schema and optimal fragments for recombination.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., supra). The conditions of temperature and ionic strength determine the “stringency” of the hybridization. For preliminary screening for homologous nucleic acids, low stringency hybridization conditions, corresponding to a Tm (melting temperature) of 55° C., can be used, e.g., 5× SSC, 0.1% SDS, 0.25% milk, and no formamide; or 30% formamide, 5× SSC, 0.5% SDS). Moderate stringency hybridization conditions correspond to a higher Tm, e.g., 40% formamide, with 5× or 6× SCC. High stringency hybridization conditions correspond to the highest Tm, e.g., 50% formamide, 5× or 6× SCC. SCC is a 0.1 SM NaC 1, 0.01 SM Na-citrate. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived (see Sambrook et al., supra, 9.50-9.51). For hybridization with shorter nucleic acids, i.e., oligonucleotides, the position of mismatches becomes more important, and the length of the oligonucleotide determines its specificity (see Sambrook et al., supra, 11.7-11.8). A minimum length for a hybridizable nucleic acid is at least about 10 nucleotides; preferably at least about 15 nucleotides; and more preferably the length is at least about 20 nucleotides.

Unless specified, the term “standard hybridization conditions” refers to a Tₘ of about 55° C., and utilizes conditions as set forth above. In a preferred embodiment, the Tₘ is 60° C.; in a more preferred embodiment, the Tₘ is 65° C. In a specific embodiment, “high stringency” refers to hybridization and/or washing conditions at 68° C. in 0.2× SSC, at 42° C. in 50% formamide, 4× SSC, or under conditions that afford levels of hybridization equivalent to those observed under either of these two conditions.

Suitable hybridization conditions for oligonucleotides (e.g., for oligonucleotide probes or primers) are typically somewhat different than for full-length nucleic acids (e.g., full-length cDNA), because of the oligonucleotides' lower melting temperature. Because the melting temperature of oligonucleotides will depend on the length of the oligonucleotide sequences involved, suitable hybridization temperatures will vary depending upon the oligonucleotide molecules used. Exemplary temperatures may be 37° C. (for 14-base oligonucleotides), 48° C. (for 17-base oligonucleotides), 55° C. (for 20-base oligonucleotides) and 60° C. (for 23-base oligonucleotides). Exemplary suitable hybridization conditions for oligonucleotides include washing in 6× SSC/0.05% sodium pyrophosphate, or other conditions that afford equivalent levels of hybridization.

The term “isolated” means that the referenced material is removed from the environment in which it is normally found. Thus, an isolated biological material can be free of cellular components, i.e., components of the cells in which the material is found or produced. In the case of nucleic acid molecules, an isolated nucleic acid includes a PCR product, an isolated mRNA, a cDNA, or a restriction fragment. In another embodiment, an isolated nucleic acid is preferably excised from the chromosome in which it may be found, and more preferably is no longer joined to non-regulatory, non-coding regions, or to other genes, located upstream or downstream of the gene contained by the isolated nucleic acid molecule when found in the chromosome. In yet another embodiment, the isolated nucleic acid lacks one or more introns. Isolated nucleic acid molecules include sequences inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified.

The term “purified” refers to material that has been isolated under conditions that reduce or eliminate the presence of unrelated materials, i.e., contaminants, including native materials from which the material is obtained. For example, a purified protein is preferably substantially free of other proteins or nucleic acids with which it is associated in a cell; a purified nucleic acid molecule is preferably substantially free of proteins or other unrelated nucleic acid molecules with which it can be found within a cell. The term “substantially free” is used operationally, in the context of analytical testing of the material. Preferably, purified material substantially free of contaminants is at least 50% pure; more preferably, at least 90% pure, and more preferably still at least 99% pure. Purity can be evaluated by chromatography, gel electrophoresis, immunoassay, composition analysis, biological assay, and other methods known in the art.

Methods for purification are well-known in the art. For example, nucleic acids can be purified by precipitation, chromatography (including preparative solid phase chromatography, oligonucleotide hybridization, and triple helix chromatography), ultracentrifugation, and other means. Polypeptides and proteins can be purified by various methods including, without limitation, preparative disc-gel electrophoresis, isoelectric focusing, HPLC, reversed-phase HPLC, gel filtration, ion exchange and partition chromatography, precipitation and salting-out chromatography, extraction, and countercurrent distribution. For some purposes, it is preferable to produce the polypeptide in a recombinant system in which the protein contains an additional sequence tag that facilitates purification, such as, but not limited to, a polyhistidine sequence, or a sequence that specifically binds to an antibody, such as FLAG and GST. The polypeptide can then be purified from a crude lysate of the host cell by chromatography on an appropriate solid-phase matrix. Alternatively, antibodies produced against the protein or against peptides derived therefrom can be used as purification reagents. Cells can be purified by various techniques, including centrifugation, matrix separation (e.g., nylon wool separation), panning and other immunoselection techniques, depletion (e.g., complement depletion of contaminating cells), and cell sorting (e.g., fluorescence activated cell sorting or FACS). Other purification methods are possible. A purified material may contain less than about 50%, preferably less than about 75%, and most preferably less than about 90%, of the cellular components with which it was originally associated. The “substantially pure” indicates the highest degree of purity which can be achieved using conventional purification techniques known in the art.

In preferred embodiments, the terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

Molecular Physics

The term “sequence space” refers to the set of all possible sequences of residues for a polymer having a specified length. Thus, for example, the sequence space for a protein or polypeptide 300 amino acid residues in length is the group consisting of all sequences of 300 amino acid residues, e.g. 20³⁰⁰=10³⁹⁰ sequences of 300 amino acids. Similarly, the sequences space of a nucleic acid 300 nucleotides in length is the group consisting of all sequences of 300 nucleotides, etc.

“Conformational energy” refers generally to the energy associated with a particular “conformation”, or three-dimensional structure, of a polymer, such as the energy associated with the conformation of a particular protein or nucleic acid. Interactions that tend to stabilize a macromolecule such as a polymer (e.g., a protein or nucleic acid) have energies that are quantitatively represented in this specification as negative energy values, whereas interactions that destabilize a polymer have positive energy values. Thus, the conformational energy for any stable polymer is quantitatively represented by a negative conformational energy value. Generally, the conformational energy for a particular polymer will be related to that polymer's stability. In particular, polymers and other macromolecules that have a lower (i.e., more negative) conformational energy are typically more stable, e.g., at higher temperatures (i.e., they have greater “thermal stability”). Accordingly, the conformational energy of a polymer may also be referred to as the polymer's “stabilization energy”.

Typically, the conformational energy is calculated using an energy “force-field” that calculates or estimates the energy contribution from various interactions which depend upon the conformation of a polymer. The force-field is comprised of terms that include the conformational energy of the alpha-carbon backbone, side chain—backbone interactions, and side chain-side chain interactions. Typically, interactions with the backbone or side chain include terms for bond rotation, bond torsion, and bond length. The backbone-side chain and side chain-side chain interactions include van der Waals interactions, hydrogen-bonding, electrostatics and solvation terms. Electrostatic interactions may include coulombic interactions, dipole interactions and quadrapole interactions). Other similar terms may also be included. Force-fields that may be used to determine the conformational energy for a polymer are well known in the art and include the CHARMM (see, Brooks et al., J. Comp. Chem. 1983,4:187-217; MacKerell et al., in The Encyclopedia of Computational Chemistry, Vol. 1:271-277, John Wiley & Sons, Chichester, 1998 ), AMBER (see, Cornell et al., J. Amer. Chem. Soc. 1995, 117:5179; Woods et al., J. Phys. Chem. 1995, 99:3832-3846; Weiner et al., J. Comp. Chem. 1986, 7:230; and Weiner et al., J. Amer. Chem. Soc. 1984, 106:765) and DREIDING (Mayo et al., J. Phys. Chem. 1990, 94:8897) force-fields, to name a few.

In a preferred implementation, the hydrogen bonding and electrostatics terms are as described in Dahiyat & Mayo, Science 1997 278:82). The force field can also be described to include atomic conformational terms (bond angles, bond lengths, torsions), as in other references. See e.g., Nielsen J E, Andersen K V, Honig B, Hooft R W W, Klebe G, Vriend G, & Wade R C, “Improving macromolecular electrostatics calculations,” Protein Engineering, 12: 657662(1999); Stikoff D, Lockhart D J, Sharp K A & Honig B, “Calculation of electrostatic effects at the amino-terminus of an alpha-helix,” Biophys. J., 67: 2251-2260 (1994); Hendscb ZS, Tidor B, “Do salt bridges stabilize proteins—a continuum electrostatic analysis,” Protein Science, 3: 211-226 (1994); Schneider J P, Lear J D, DeGrado W F, “A designed buried salt bridge in a heterodimeric coil,” J. Am. Chem. Soc., 119: 5742-5743 (1997); Sidelar C V, Hendsch Z S, Tidor B, “Effects of salt bridges on protein structure and design,” Protein Science, 7: 1898-1914 (1998). Solvation terms could also be included. See e.g., Jackson S E, Moracci M, elMastry N, Johnson C M, Fersht A R, “Effect of Cavity-Creating Mutations in the Hydrophobic Core of Chymotrypsin Inhibitor 2,” Biochemistry, 32: 11259-11269 (1993); Eisenberg, D & McLachlan A D, “Solvation Energy in Protein Folding and Binding,” Nature, 319: 199-203 (1986); Street A G & Mayo S L, “Pairwise Calculation of Protein Solvent-Accessible Surface Areas,” Folding & Design, 3: 253-258 (1998); Eisenberg D & Wesson L, “Atomic solvation parameters applied to molecular dynamics of proteins in solution,” Protein Science, 1: 227-235 (1992); Gordon & Mayo, supra.

“Coupled residues” are residues in a polymer that interact, through any mechanism. The interaction between the two residues is therefore referred to as a “coupling interaction”. Coupled residues generally contribute to polymer fitness through the coupling interaction. Typically, the coupling interaction is a physical or chemical interaction, such as an electrostatic interaction, a van der Waals interaction, a hydrogen bonding interaction, or a combination thereof. As a result of the coupling interaction, changing the identity of either residue will affect the fitness of the polymer, particularly if the change disrupts the coupling interaction between the two residues. Coupling interactions may also preferably be described by a distance parameter between residues in a polymer. If the residues are within a certain cutoff distance, they are considered interacting. This approach provides good results and can be computed relatively quickly.

If a coupling interaction is considered disrupted by crossover recombination, a “crossover disruption” (E_{c}) parameter for each mutant can be determined. The “crossover disruption” (E_{c}) of a mutant is determined by the number of disrupted coupled interactions caused by the crossover from one sequence to another. Coupled, pairwise interactions between amino acids from different parent sequences are summed, while the interactions within fragments and shared between fragments from the same parent are not counted. Candidate or optimal crossover locations on genes correspond to locations that permit recombination with minimal disruption of coupling interactions, e.g. without disrupting parental clusters of favorably interacting DNA residues (building blocks or schema) in the parental genes.

A “crossover disruption profile” is the crossover disruption that would result if a crossover occurred at a given residue (or each residue) of a biopolymer sequence. The term “crossover” refers to a recombination process in which an exchange of polymer sequences occurs between two linear polymer sequences, e.g. any point at which the genetic material from two parents is switched in an offspring.

A “schema disruption” is the disruption of a set of residues that interact in a collectively beneficial way. For example, it may be harmful to the recombinant mutant sequences if the residues participating in a schema come from different parents. Schema disruption is a combination of the disruption of independent structural elements (domains) or structural elements that cause a breaking of coupling interactions. See e.g., Holland, Adaptation in Natural and Artificial Systems, University of Michigan Press, Ann Arbor, Mich. (1975).

Thus, schema are clusters of amino acids in the structure that interact in some positive way. For example, they may interact through hydrogen-bonds to stabilize the structure or they may interact to perform the catalytic function of a protein (enzyme). When these clusters of interacting residues are separated by recombination (because some come from one parent and others come from a different parent), this has a detrimental effect on the protein—e.g. by destabilizing it, or making it non-functional. An objective of the invention is to minimize and prevent schema disruption, e.g. by modeling the recombination of parent fragments to preserve schema in the resulting mutants.

A “domain disruption” is the disruption of a compact structural domain or folding unit of a biopolymer, e.g. a protein.

Schema disruption and domain disruption may also be profiled, in a manner akin to crossover disruption profiles.

The “crossover probability”, which is also denoted here by the symbol P_{c}, is the probability that a crossover will occur between two given nucleic or amino acid sequences (for example, between two homologous genes). Crossover probability is related to the experimental average fragment size in recombination experiments, and is a parameter that can be influenced or controlled in certain recombination protocols. For example, crossover probability can be controlled in DNA shuffling according to the time that parental templates are exposed to the DNA-cleaving DNAse. In StEP recombination, this is controlled by timing the annealing/extension cycles. The relationship between fragment size f and crossover probability P_{c} can be expressed as: P_{c}=(f−1)/N, where N is either the number of amino acid residues (when calculating recombinant mutants based on a protein sequence), or the number of nucleotides (when calculating the recombinants based on the DNA sequence).

The terms “crossover location” and “cut-point” are synonymous. The term refers to the location on a biopolymer sequence where recombination occurs. A cut point is a specific position at which a polymer sequence is broken in recombination.

The term “crossover region” refers to the area surrounding the crossover location, for example within a range of residues on either side of a cut point. In certain experiments and recombination methods the precise location of a cut point is uncertain or cannot be determined or experimentally resolved. For example, when two parents share sequence identity, it may not be possible to determine from the sequence of the recombinant offspring precisely where within an aligned or surrounding region the cut point (crossover) occurred. The range of possible cut points, each of which could have produced the observed recombination results, can be called the crossover region. Once a region of sequence identity (a crossover region) has been identified, the specific placement of the cut point is not critical.

The term “fitness” is used to denote the level or degree to which a particular property or combination of properties for a polymer (e.g. a biopolymer such as a protein or a nucleic acid) is optimized. In directed evolution methods of the invention, the fitness of a polymer is preferably determined by properties which are identified for improvement. For example, the fitness of a protein may refer to the protein's stability (e.g. at different temperatures or in different solvents), its biological activity or efficiency (e.g. catalytic function), its binding affinity or selectivity (e.g. enantioselectivity), its solubility (e.g. in aqueous or organic solvent), and the like.

Fitness can be determined or evaluated experimentally or theoretically, e.g. computationally. Other examples of fitness properties include enantioselectivity, activity towards non-natural substrates, and alternative catalytic mechanisms. Coupling interactions can be modeled as a way of evaluating or predicting fitness.

Preferably, the fitness is quantitated so that each polymer (e.g., each amino acid or nucleotide sequence) will have a particular “fitness value”. For example, the fitness of a protein may be the rate at which the polymer catalyzes a particular chemical reaction, or the protein's binding affinity for a ligand. In another embodiment, the fitness of a polymer refers to the conformational energy of the polymer and is calculated, e.g., using any method known in the art.

Generally, the fitness of a polymer is quantitated so that the fitness value increases as the property or combination of properties is optimized. For example, where the thermal stability of a polymer is to be optimized (conformational energy is preferably decreased), the fitness value may be the negative conformational energy; i.e., F=−E.

Such techniques are found in the following exemplary references: Brooks B. R., Bruccoleri R E, Olafson, B D, States D J, Swarninathan S & Karplus M, “CHARMM: A Program for Macromolecular Energy, Minimization, and Dynamics Calculations,” J. Comp. Chem., 4: 187-217 (1983); Mayo S L, Olafson B D & Goddard W A G, “DREIDING: A Generic Force Field for Molecular Simulations,” J. Phys. Chem., 94: 8897-8909 (1990); Pabo C O & Suchanek E G, “Computer-Aided Model-Building Strategies for Protein Design,” Biochemistry, 25: 5987-5991 (1986); Lazar G A, Desjarlais J R & Handel T M, “De Novo Design of the Hydrophobic Core of Ubiquitin,” Protein Science, 6: 1167-1178 (1997); Lee C & Levitt M, “Accurate Prediction of the Stability and Activity Effects of Site-Directed Mutagenesis on a Protein Core,” Nature, 352: 448-451 (1991); Colombo G & Merz K M, “Stability and Activity of Mesophilic Subtilisin E and Its Thermophilic Homolog: Insights from Molecular Dynamics Simulations,” J. Am. Chem. Soc., 121:6895-6903 (1999); Weiner S J, Kollman P A, Case D A, Singh U C, Ghio C, Alagona G, Profeta S J, Weiner P, “A new force field for molecular mechanical simulation of nucleic acids and proteins,” J. Am. Chem. Soc., 106: 765-784 (1984).

The term “fitness landscape” is used to describe the set of all fitness values belonging to all polymer sequences in a sequence space. Thus, for example, referring again to the sequence space for proteins 300 amino acid residues in length (i.e., the group consisting of all sequences of 300 amino acid residues), each polypeptide in the sequence space will have a particular fitness value that may (at least in theory) be calculated or measured (e.g., by screening each polypeptide to determine its fitness). The set of these fitness values is therefore the fitness landscape of the sequence space for proteins 300 amino acid residues in length. In many embodiments fitness values may vary considerably among individual sequences in a given sequence space. The fitness value for a given sequence may be higher or lower than other, similar sequences in the sequence space. These fitness values are therefore referred to as “local maxima” (or “local optima”) and “local minima”, respectively. Such a fitness landscape is described as “rugged” when it contains many local maxima and/or local minima in the fitness values. In the all representations of the fitness landscape, there is a “global optimum,” representing the sequence with the highest fitness. If the highest fitness is degenerate (multiple sequence have the same fitness), then more than a single sequence can be the global optimum. An objective of directed evolution and computational design methods is to generate sequences having fitness values greater than the fitness value(s) of the starting (e.g. parent) sequence or sequences. In a preferred embodiment of the invention, the directed evolution and computational design methods generate sequences having fitness values as close to the global optimum as is possible.

The “fitness contribution” of a polymer residue refers to the level or extent f(iₐ) to which the residue iₐ, having an identity a, contributes to the total fitness of the polymer. Thus, for example, if changing or mutating a particular polymer residue will greatly decrease the polymer's fitness, that residue is said to have a high fitness contribution to the polymer. By contrast, typically some residues iₐ in a polymer may have a variety of possible identities a without affecting the polymer's fitness. Such residues, therefore have a low contribution to the polymer fitness.

5.2 General Methods

In accordance with the invention, there may be employed conventional molecular biology, microbiology and recombinant DNA techniques within the skill of the art. Such techniques are explained fully in the literature. See, for example, Sambrook, Fitsch & Maniatis, Molecular Cloning: A Laboratory Manual, Second Edition (1989) Cold Spring Harbor Laboratory Press, Cold Spring Harbor, N.Y. (referred to herein as “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins, eds. 1984); Animal Cell Culture (R. I. Freshney, ed. 1986); Immobilized Cells and Enzymes (IRL Press, 1986); B. E. Perbal, A Practical Guide to Molecular Cloning (1984); F. M. Ausubel et al. (eds.), Current Protocols in Molecular Biology, John Wiley & Sons, Inc. (1994).

The invention pertains to a computational method for identifying cut points or locations in proteins that will permit crossovers in in vitro recombination experiments, while retaining structural stability (and consequently, desirable properties) in the offspring hybrid proteins. The invention can be applied to protein sequences of any or no sequence similarity. Sequence and tertiary structural information at the protein level for at least one of the starting parental sequences is used to identify structural domains or coupled residues and calculate their disruption.

5.3 Overview of Modeling Techniques

Disruption Profiles.

According to the invention, recombination modeling calculations are applied to determine the disruption of a biopolymer fragment (e.g. a schema disruption profile and/or a crossover disruption profile) relative to the remainder of the structure. In other words, what structural changes produced by recombination are compatible with a parent or a functional “starting” or “reference” structure? In this way, recombinations (and recombinants) that are predicted to disrupt schema (or coupling interactions) can be eliminated in favor of a smaller library of recombinants predicted to preserve them. This library is more likely to contain offspring which retain essential and/or beneficial properties(such as activity and stability) and can be searched for other or improved properties relative to their parents. The techniques for determining disruption profiles include: (a) calculation of crossover disruption, e.g. using distance-based or energy based criteria for coupling; (b) calculation of domains in the protein structure; and (c) calculating the disruption (e.g. a disruption profile) based on a crossover disruption, domain disruption, or both. A schema disruption based on a combination of the domain and crossover disruption is preferred. Distance-based criteria for crossover disruption of coupling is also preferred.

Recombination Modeling.

Calculations are made to model possible parental fragments for recombination based on: (a) a requirement of sequence identity between parents (for sequence-identity-dependent experimental protocols, such as DNA shuffling); (b) a constraint on the number and location of crossovers (for example, the ITCHY protocol allows a single cut point, which very substantially reduces the number ofpossible fragments; (c) other specified constraints, e.g., exon shuffling; and/or (d) a protocol without constraints (used to determine the optimal crossover).

5.4 Schema Disruption Model

Interactions among residues of a biopolymer can be modeled as schema, which in turn can be evaluated (e.g. in a schema disruption profile) to determine optimum crossover locations for recombining two or more parent molecules. Schema can be based on coupling interactions between residues, e.g. based on conformational energy and/or interatomic distances. According to the invention, crossover locations that do not disrupt coupling interactions or schema are preferred.

Principles of crossover disruption of coupling interactions according to the invention are illustrated in FIG. 2. A “Protein Z” having amino acid residues (shown as circles) at positions 1 through 12 is shown in cartoon form. In part A of FIG. 2, Protein Z is shown in a folded cartoon at the left, and in a two-dimensional representation of its folded three- dimensional conformation at the right. These drawings indicate the relative location or position in space of each residue with respect to the other residues. The black line represents peptide bonds between the residues 1-12. The grey dotted lines represent coupling interactions between amino acid side chains. For example, residue 3 is joined to residues 2 and 4 bypeptide bonds (solid lines). Residue 3 is coupled to residues 11 and 12 by coupling interactions (dotted lines), which may be associated with any molecular forces other than the peptide bonds of the protein's primary structure.

The coupling interactions can be mapped to a coupling matrix, as shown for example in part B of FIG. 2, In this view of the matrix, the primary amino acid sequence 1-12 is shown in linear form, with each superimposed line indicating a coupling interaction. The number of interactions affecting each residue is conveniently shown. These lines also show which residues are coupled to each other.

According to the invention it is desirable for recombination to minimize disruption of coupling interactions. This can be achieved, for example, by cutting the sequence for recombination at locations selected so that the least number of interactions are separated onto different fragments. Desirable or optimum cut points can be identified with the aid of a crossover interaction profile, or of a crossover disruption (E_{c}), as shown graphically in part C of FIG. 2. The graph shows the crossover disruption E_{c}, or the number of coupling interactions that are broken (y-axis), for each residue of the protein (x-axis), when a single cut is located before each residue. (A cut point can be named for the residue it follows or proceeds. In this example, each cut point occurs before the named residue.) For example, if Protein Z is cut at residue 3 in a recombination experiment, i.e. the cut is between residues 2 and 3, the resulting fragments for recombination (e.g. from two different parent proteins) are a fragment 1-2 and a fragment 3-12. (Note that in the example recombination actually occurs at the genetic level, at a corresponding cut point in a nucleotide sequence that encodes Protein Z.) The graph C of FIG. 2, line the diagram B, show that for this hypothetical protein Z, a cut point at residue 3 will disrupt seven coupling interactions. Crossover disruption can be calculated by computer, using known programming methods.

For the simple structure of Protein Z, the graph shows that the crossover disruption is greatest if a cut is made in the center of the gene, e.g. at a nucleotide triplet or codon corresponding to one of the amino acid residues 4-8. According to the invention, cut points are selected to minimize crossover disruption, so there is a bias in this example toward selecting cut points at the ends or termini of Protein Z. For example, a cut point at residue 11 (e.g. parent A donates residues 1-10 and parent B donates residues 11-12) will produce mutants having less crossover disruption than a cut point at residue 6 (parent A donates residues 1-5 and parent B donates residues 6-12). Mutants with less crossover disruption are more likely to be functional and retain desirable properties from one or both parents. When such parents are used in directed evolution experiments, the probability of adding or improving desirable properties, without loss of stability, utility or functionality, is increased. In this way the methods of the invention are not random. Cut points for recombination are not obtained only as a random consequence of known directed evolution methods, such as error-prone PCR, family shuffling or StEP. Rather, more favorable or promising cut points are identified and preselected according to an evaluation of coupling interactions and crossover disruption, as illustrated in the coupling matrix of FIG. 2.

The invention is not limited to the use of a single cut point. More than one cut point may be used to provided a plurality of fragments from two or more parents for recombination. For example, two cut points can be selected for hypothetical Protein Z, indicated by scissor icons in part B of FIG. 2. When the residues between these cut points come from parent A (residues 4-7) and the terminal fragments come from parent B (residues 1-3 and 8-12) the crossover disruption is reduced to zero. According to the invention, these cut points and the resulting parental fragments would be preferred for recombination experiments, e.g. where mutants obtained from such recombinations are screened for desirable properties, including new or modified properties, or the loss or reduction of one or more undesirable properties.

Calculation of Coupling Interactions from a Crystal Structure

As shown by FIG. 1B, a structure file of a parent polymer is obtained, such as a data file representing the three-dimensional structure of a gene or a protein. Databases of this kind are known in the art. Coupling interactions between the building blocks of the polymer are then identified from the structural data, using the methods described herein. From the identified coupling interactions, structural domains, or compact units of structure, can be identified and represented as a schema for the polymer. For example, when the polymer is a protein, and the schema building blocks are amino acid residues, the set of residues contributing to each domain of the three-dimensional protein structure can be determined. Because a protein is folded, the residues which interact and participate in a domain may and often are not adjacent to each other in the linear or primary sequence of the protein. This is shown, for example, in the cartoon for “Protein Z” in FIG. 2A, where amino acid residues 1 and 8 are close to each other in the three dimensional structure.

Domains, for example folding domains, can be identified by testing for residues which interfere with structural stability, and which form groups of residues that are considered essential or important to stability, based on threshold criteria as described herein (e.g. conformational energy or atomic distance thresholds). Groups of residues which, if altered, would significantly impair structural stability are identified as domains. Crossover disruptions can be calculated for the residues, using the methods described herein, to identify domains and generate schema profile. See e.g., the accompanying Examples, and especially Example 6.3, for domain identification, and schema and crossover disruption based on distance criteria.

Once the domains and sets of interacting building blocks are identified and a schema is determined, a crossover disruption E_{c} is determined for each domain. The results for all domains of the polymer can be plotted as a schema disruption profile, as described herein, and in a manner similar to a crossover disruption profile. To determine the crossover disruption and generate a profile, a threshold disruption value is set. The contribution of each residue of each domain to the structural integrity or fitness of the polymer is evaluated, based on the degree to which it interacts with each other residue of each other domain. This is compared to the threshold crossover disruption, which is determined empirically or is modeled as a probability as described above for E_{c} in a DNA shuffling recombination context. Domains which exhibit a low crossover disruption compared to the threshold are “rejected”, meaning they can be substituted without disrupting the structure. Domains which exhibit a high crossover disruption are “accepted”, meaning that they are schema which should be preserved in the offspring. This follows from the principles described above. Domains which are essential or important to the structural integrity or shape of the polymer (which have a high crossover disruption) should not be disrupted by recombination, in favor of crossovers in domains that are less essential or important to the structural integrity or shape of the polymer (they have a low crossover disruption). It should be noted however, that the terms “accept” and “reject” (FIG. 1B) are relative, and could be interchanged, depending on the desire point of view. Thus, domains with a low crossover disruption could be “accepted” as candidates for crossover recombination. Domains with a high crossover disruption would be “rejected” for crossover recombination, so that those domains can be protected or preserved.

The process of accepting and rejecting domains to generate a schema disruption profile can be performed iteratively, until all residues of all domains are identified and their relative contribution to the structure of the polymer is determined. When this is “Done” (FIG. 1B), the data is used to mark all domains that are disruptive, so that they will be preserved—crossover recombinations in these domains will not be modeled or performed. From the remaining domains, optimal crossovers can be identified. These are the sets of possible crossovers within the low disruption domains that are calculated to perturb the polymer the least, while offering the best chances for new or improved properties.

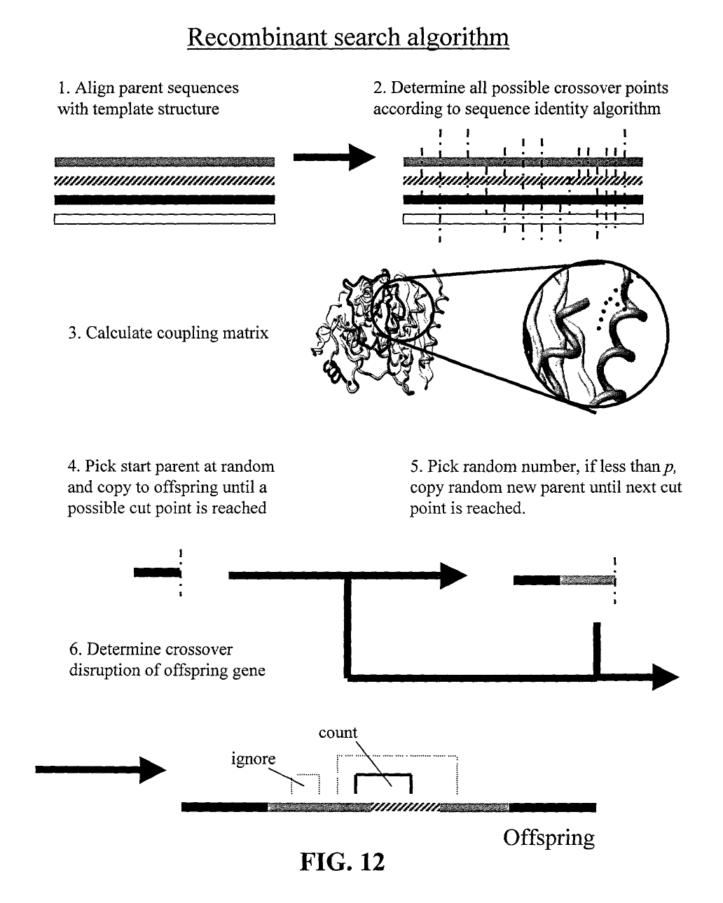

The last two steps of FIG. 1B are optional. If a recombination protocol is to be used for directed evolution experiments, the protocol may have restrictions on the crossover locations which are accessible to the method, or the number and manner in which crossovers occur. Using a cut point or fragment file which identifies and represents these restrictions, the sequence space of optimal crossovers from the previous steps can be further limited or reduced, to those which also satisfy the restrictions of the experimental protocol. For example, protocols based on homologous recombination, sequence identity or alignments, e.g. as depicted in FIG. 1A and FIG. 12, may be used in combination with the non-homologous methods described here and by reference to FIG. 1B.

Conceptually, a set of possible parents is selected based on structural similarity. In one embodiment, the parents can be identified based on regions of sequence identity. Using the computational methods described herein, a set of all possible cut points for these parents can be generated. These computations are independent of any constraints on recombination, for example limitations which may be posed by particular protocols for directed evolution. The set of optimum cut points can then be determined from the set of all possible cut points, using the methods of the invention. More particularly, cut points are selected to minimize the disruption of coupling interactions in the three-dimensional structure of the protein. Recombination or evolution methods can then be selected and adapted to cut and recombine the parents at the selected cut points.

In preferred methods of the invention, once the parental sequences are aligned and candidate cut points identified, the structure or conformation of one of the parent sequences is also obtained or otherwise provided (FIG. 1A). The preferred method of the invention requires the structure or conformation of a parental amino acid be obtained or otherwise provided. In many preferred embodiments, and particularly in embodiments where the parent sequence is the sequence for a known protein or nucleic acid, the structure or conformation of the parent sequence will be known and can be obtained from any of a variety of resources (for a review, see Hogue et al., Methods Biochem. Anal. 1998, 39:46-73). For example, and not by way of limitation, the Protein Data Bank (PDB) (Berman et al., Nucl. Acids Res. 2000, 28:235-242) is a public repository of three-dimensional structures for a large number of macromolecules, including the structures of many proteins, nucleic acids and other biopolymers.

Alternatively, in many embodiments the structure of a polymer (e.g., protein) sequence that is similar or homologous to the parent sequence will be known. In such instances, it is expected that the conformation of the parent sequence will be similar to the known structure of the homologous polymer. The known structure may, therefore, be used as the structure for the parent sequence or, more preferably, may be used to predict the structure of the parent sequence (i.e., in “homology modeling”). As a particular example, the Molecular Modeling Database (MMDB) (see, Wang et al., Nucl. Acids Res. 2000, 28:243-245; Marchler-Bauer et al., Nucl. Acids Res. 1999, 27:240-243) provides search engines that may be used to identify proteins and/or nucleic acids that are similar or homologous to a parent sequence (referred to as “neighboring” sequences in the MMDB), including neighboring sequences whose three-dimensional structures are known. The database further provides links to the known structures along with alignment and visualization tools whereby the homologous and parent sequences may be compared and a structure may be obtained for the parent sequence based on such sequence alignments and known structures.

In other embodiments, where the structure for a particular parent sequence may not be known or available, it is typically possible to determine the structure using routine experimental techniques (for example, X-ray crystallography and Nuclear Magnetic Resonance (NMR) spectroscopy) and without undue experimentation. See, e.g., NMR of Macromolecules: A Practical Approach, G. C. K. Roberts, Ed., Oxford University Press Inc., New York (1993). Alternatively, and in less preferable embodiments, the three-dimensional structure of a parent sequence may be calculated from the sequence itself and using ab initio molecular modeling techniques already known in the art. Three-dimensional structures obtained from ab initio modeling are typically less reliable than structures obtained using empirical (e.g., NMR spectroscopy or X-ray crystallography) or semi-empirical (e.g., homology modeling) techniques. However, such structures will generally be of sufficient quality, although less preferred, for use in the methods of this invention.

Calculation of a Schema Disruption Profile

Once the three dimensional amino structure of one of the parental sequences is determined, the method of the invention provides for the determination of coupling interactions between pairwise amino acid side chains. In a preferred embodiment the coupling interactions are represented by the use of a coupling matrix, as described infra. A matrix can be presented diagrammatically, or its members can be described in numerical or binary fashion. For example, if residues 3 and 8 of a structure are the only coupled residues, then the (3,8) and (8,3) members or cells of the N×N matrix can be set to 1, and all other cells are set to 0.

The coupling interactions can be defined by the determination of conformational energy between residues, or based on distance parameters such as interatomic distances (the distances between atoms in residues of the polymer). Calculations based on distances are preferred. An energy or distance measure that is outside a certain threshold between residues can be used to determine that the residues are considered to be uncoupled. For example, in embodiments based on conformational energy or distance, only those residues that exhibited a stabilization or conformational energy below a defined threshold, or within a threshold interaction distance, are considered to be coupled. For example, in a preferred conformational energy embodiment, the threshold was defined as 0.25 kcal/mol.

5.5 Modeling Recombination Based on Fragment Restrictions

According to the invention, recombination protocols that limit or restrict the fragments which can recombine can be modeled, and optimal crossovers from a set or subset of fragments can be determined.

Sequence Identity Based Recombination

In this example, the invention is used to model the recombination of DNA sequences using methods that rely or depend on sequence identity. FIG. 1A provides a flow diagram illustrating a general, exemplary embodiment of the methods used in this invention. A skilled artisan can readily appreciate that certain steps may be omitted and the order of the steps may be changed. In particular, the flow diagram in FIG. 1A as well as other examples presented in Section 6, infra, describe preferred embodiments where the methods were used in directed evolution of a protein or other polypeptide. Those skilled in the art can readily appreciate, however, that the methods illustrated by these examples and throughout this specification may be used to modify any polymer or biopolymer, including any amino acid or nucleotide sequence, or any DNA or RNA molecule.

Parent Sequences.

The method shown in FIG. 1A begins with the selection of “parent” polymer sequences. For example, the parent sequences may be any amino acid sequence and may or may not correspond to a naturally occurring polypeptide. Each protein sequence is preferably associated with a nucleic acid sequence (e.g., a gene encoding the protein). A preferred embodiment utilizes homologous amino acid sequences. Another preferred embodiment utilizes non-homologous amino acid sequences. Preferably, the parent sequence is also the sequence for a protein that has some level or degree of activity or function (e.g., catalytic activity, binding affinity, solubility, thermal stability, etc.) to be optimized. The methods of the invention may then be used, e.g., to optimize the activity or function of the parent sequence and/or to optimize the activity in altered conditions. For example, in one embodiment the parent sequence may be a protein having a particular catalytic or other activity, and the methods of the invention may be used to identify sequences having the same activity but under different (generally more extreme) conditions such as conditions of temperature or of solvent (including, for example, solvent polarity, salt conditions, acidity, alkalinity, etc.). In another embodiment, the parent sequence may have a particular level or amount of activity (e.g., catalytic activity, binding affinity, etc.), and the directed evolution methods of the invention may be used to identify sequences having improved levels or amounts of that same activity (e.g., higher binding affinity or increased catalytic rate).

Align Polymer Sequences.

Once the parental sequences are selected, the sequences are aligned (FIG. 1A). The invention contemplates alignment of parental sequences in either nucleic acid or amino acid forms. In a preferred embodiment, homologous (evolutionarily related) amino acid parental sequences are aligned based upon sequence identity, sequence similarity, or a combination of both parameters. The various parameters associated with alignment of amino acid sequences is well known in the art. In another preferred embodiment, the parental sequences are aligned as nucleic acid sequences. In a preferred embodiment, the nucleic acid sequences are aligned based upon regions of sequence identity.

Alignment of parental sequences can be accomplished visually or with the use of algorithm. The invention encompasses the use of, but is not limited to, the following alignment programs: GAP, BLAST, FASTA, DNA Strider, CLUSTAL, and GCG. The invention includes the use of default parameters and standard parameters of the computer programs. It preferably includes the use of alignment parameters routinely employed in the art. A preferred embodiment of the invention utilizes BLAST amino acid alignment program to align homologous sequences. Each parent sequence is aligned with the structure sequence using a BLAST algorithm for comparing two sequences. Tatusova, T. A. & Madden T. L., FEMS Microbiol Lett. 174:247-250(1999). The BLOSUM62 matrix is used to score similar amino acids and the open gap and extension gap penalties are 11 and 1, respectively.

Determination of Possible Crossover Locations Based on Hybridization

The invention encompasses a computational “in silico” simulation of in vitro and in vivo recombination. The types of in vitro recombination that are simulated include, but are not limited to various forms of recombination methods such as, DNA shuffling, StEP, random-priming recombination, and DNAse restriction enzymes.

Crossover locations for recombination can be determined based on hybridization between parents. When parental sequences contain areas of sequence identity, aligned sequences can be examined for areas of identity based upon a predetermined subset or number of sequential identical amino acids or nucleotides in two aligned parental sequences (FIG. 1A). A preferred number of sequential identical amino acids related to the required length of the DNA for hybridization to occur in a particular recombination experiment. A preferred embodiment is to search for regions of four identical amino acids, or six identical nucleotides shared by the parents. After identification of the areas that meet the selected parameters of sequence identity a cut point in the identified area of sequence identity on the parental sequence is selected as a crossover location. The placement of the cut point within a crossover regions is not critical. As one example, the cut point may be selected at any location within the identified region of sequence identity.

In one particular embodiment of the invention, a computational algorithm was utilized to mimic DNA shuffling recombination. Starting with the aligned parental DNA sequences and their respective possible crossover locations (i.e., all possible cut points), a randomly selected parental DNA sequence served as the initial template and was copied to mutant offspring. When an identified candidate crossover location was reached in the copying process the parental template was switched to a randomly selected different parental template under specified conditions. In a preferred embodiment, the specified conditions were set as follows: (1) a randomly chosen number between 0 and 1 was less than a threshold of P_{c} (e.g. 0.03) and (2) a minimum of eight amino acids between identified crossover locations where crossovers actually occurred. The value P_{c} represents the average number of fragments that each parent gene is cut into. For example, in DNA shuffling experiments, this parameter is related to the time that the parent template DNA is exposed to the DNA- cleaving enzyme DNAse. The expression to obtain P_{c} from the average number of fragments f is P_{c}=(f−1)/N, where N is the size of the gene. The value 0.03 was set to model the fragment size reported by Stemmer, supra. for the beta-lactamase shuffling experiment.

Determining Crossover Disruption.

The computational method of the invention predicts locations on parental sequences where recombination should be most successful due to minimal disruption of tertiary amino acid interactions in a crossover mutant. A crossover disruption E_{c} for each mutant is determined. In one embodiment of the invention, coupling interactions are considered disrupted if one of the amino acid pairs of an interacting pairs is replaced with an amino acid from a different parent sequence in the hybrid mutant protein. The crossover disruption for a particular mutant is determined by the summation of all coupled interactions that are considered disrupted.

Election of a Crossover Mutant with Minimal Crossover Disruption.

Once the crossover disruption for a pool of mutant biopolymers is determined, a threshold is applied to screen the mutant biopolymers for those mutants that exhibit minimal amounts of crossover disruption. Non-limiting examples of selection parameters include the following: (1) an application of a threshold, (2) selection of 10% of the mutant pool that exhibited the least amount of crossover disruption, (3) selection of the 10 mutants that exhibited the least amount of disruption, (4) selection of crossover mutants exhibiting a crossover disruption below an average value, (5) selection of crossover mutants exhibiting crossover disruption below a first standard deviation or more. In a preferred embodiment of the method, a threshold is applied such that 1% of the total mutant pool is allowed by the threshold. In another embodiment of the invention, a more stringent threshold is utilized, whereby only 0.001% of the pool is allows by the threshold.

A variation of this method, as depicted in the flow chart of FIG. 1A is shown diagrammatically in FIG. 12.

Non-sequence Identity Based Recombination

Recombination that is not dependent on sequence identity can be also be modeled according to the invention. This can be called “non-homologous” recombination. Schema based on structural features of parent polymers are identified, such as three-dimensional domains of a protein, and accordingly, it is not necessary to align parent polymers in this approach.

Other recombination methods limit the number of fragments and the locations for crossovers between the parents. For example, the ITCHY protocol limits recombination to one crossover point. Other known protocols use restriction enzymes to cut at very specific locations in the gene, based on a stretch of DNA sequence 3-5 nucleotides long. If restriction enzymes are used to fragment the parents, then crossovers occur based on the set of restriction enzymes chosen by the researcher. For example, if a restriction enzyme is chosen that only cuts at ATGG, then crossovers can only occur where ATGG appears in the parental DNA sequence.

Further, methods based on limiting the fragments to the recombination of exons can be employed. (Exons are naturally occurring fragments of the gene that precede the splicing step of transcription). By restricting the fragments, the potential locations of crossovers are restricted. The restrictions that result from these methods can be included in the calculations and computation described here, for example by noting the potential crossover points and either reconstructing possible chimeric mutants, as described infra, or by noting the location of these crossover points with respect to the disruption of schema.

The schema disruption calculation provides a guide for both the restriction-enzyme-based and exon-based recombination methods. From a starting database of exons or restriction enzymes, a subset can be chosen that generate crossover locations that minimize the schema disruption. This subset has a higher likelihood of generating chimeric mutants that are structurally stable, thus generating libraries where improvement in the desired properties are more likely.

5.6 Directed Evolution Methods to Target Optimal Crossover Locations

The methods described above are particularly useful for directed evolution experiments, e.g., to obtain proteins, nucleic acids or other polymers having one or more desirable properties. For example, the computational models and protein design algorithms can be used with directed evolution techniques to target mutants or hybrids within a subset of the total sequence space, and particularly within a sequence space corresponding to higher fitness probabilities. Accordingly, the invention provides genetic engineering methods, including methods of directed evolution, for obtaining polymers that have one or more improved properties. The improved properties include any property or combination of properties that can be detected by a user and include, for example, properties of catalytic activity (for example, increased rates of catalysis), properties of stability (for example, increased thermal stability) or properties of binding affinity (for example, increased affinity for a particular ligand or increased affinity for a substrate) to name a few. Preferably the desirable property is a property that can be detected in a screening assay.

Mutagenesis and Recombination

In general, directed evolution methods comprise selecting at least one polymer sequence. The polymer sequence is preferably the sequence for a biopolymer (e.g., a nucleic acid or a polypeptide) that has a particular property or properties of interest. For example, the particular property of the parent may be a particular catalytic activity, binding to a particular substrate or ligand, thermal stability or a combination thereof. Preferably the property is one that can be readily determine or evaluated by a screening assay, e.g. a high throughput screen. One or more residues of the parent polymer sequence is then selected or targeted for mutation. In traditional methods for directed evolution, selection is random. For example, all or a large fraction of the residues are available and/or are selected, e.g., by error prone PCR or DNA shuffling. However, in the directed evolution methods of the invention, specific residues in the parent sequence are identified as candidate crossover locations. The crossover locations may be identified, for example, according to the analytical methods described above.

One or more, and preferably a plurality of mutant polymer sequences may then be generated based on the parent sequence. In particular, the directed evolution methods of the invention preferably generate a plurality of mutants which are identical to the parent sequence except that one or more structurally tolerant residues are mutated. Polymers having the mutant sequences may then be generated using polymer synthesis and or recombinant technologies well known in the art, and the polymers having these mutant sequences are then preferably screened for the one or more properties of interest. In particular, methods of directed evolution typically have, as their goal, the selection and/or identification of polymers (in particular, modified polymers) wherein one or more particular properties of interest are altered, and are preferably improved. For example, a directed evolution method may have, as its goal, the selection of polymers that have improved catalytic activity (e.g., a higher rate of catalysis), improved (e.g., stronger) binding to a particular ligand or substrate, or greater thermal stability. Therefore, in preferred embodiments one or more of the mutant polymers are selected where one or more of the properties of interest are different from the parent sequence. Preferably, the one or more properties of interest are improved in the selected polymer sequences.

In preferred embodiments, methods of directed evolution may be repeated to generate and identify polymers where one or more properties of interest progressively improve with each iteration. Accordingly, in a preferred embodiment, one or more of the selected polymers may be selected as a new parent sequence, for use in a next round of iteration in the directed evolution method. Crossover locations in the new parent sequence may then be identified and selected, and a second generation of mutants can be generated and screened as described above. Improved mutants may also be recombined if desired, using conventional genetic engineering techniques, to obtain further variations and improvements. These processes may be repeated as desired, to obtain successive generations of mutants.

Polymer Evolution Techniques

Methods for the directed evolution of polymers such as nucleic acids and polypeptides are well known in the art. See, for example, Dube et al., Gene, 137:41 (1993); Moore & Arnold, Nature Biotechnology, 14:458 (1996); Joo et al., Nature, 399:670 (1999); Zhao & Arnold, Protein Engineering, 12:47(1999); Skandalis et al., Chem. Biol., 4:889-898 (1997); Nikolova et al., Proc. Natl. Acad. Sci. USA., 95:14675 (1998); Miyazaki & Arnold, J. Molecular Evolution, 49:716 (1999). See, also, U.S. Pat. Nos. 5,741,691 and 5,811,238; International Patent Applications WO 98/42832, WO 95/22625, WO 97/20078, WO 95/41653, and U.S. Pat. Nos. 5,605,793 and 5,830,721. Generally, such methods work by selecting a parent sequence, typically a particular protein, and generating large numbers of mutants, for example by error prone PCR of a gene encoding the selected protein. The mutants are then tested, preferably in a screening assay, to identify mutants that actually have an improved property detected in the assay (for example, increased catalytic activity, or stronger binding to a ligand or substrate). These mutants are selected and again mutated, and the second generation of mutants is again tested to identify new mutants where the property is further improved. Thus, traditional directed evolution methods randomly search through the sequence space of a polymer one residue at a time to identify mutants with an increased fitness.

Such traditional methods are limited, however, by the finite capacity of existing assays to screen mutants. Existing screening assays may observe and/or select from between about 10³ or 10¹² mutants, depending on the particular method. However, for a typical protein of 300 amino acid residues the number of possible amino acid combinations is about 1×10³⁰. Thus, screening assays can only observe a small fraction of sequences in the sequence space of a given parent.

Using the analytical methods described above, a user can improve upon such existing methods by identifying locations on polymers that allow crossovers to occur while maintaining their function and specifically selecting those locations for mutation in the iterative step of a directed evolution experiment. In preferred embodiments a user may identify and target residues that have crossover locations that exhibit crossover disruption below a certain value in in vitro experiments.

The invention encompasses, but is not limited to, the following examples of in vitro techniques: (1) fragmentation and reassembly techniques (e.g. the Stemmer DNA shuffling method, Stemmer, Nature 1994, 370:389; (2) staggered extension process (StEP)(Zhao et al., Nature Biotechnology 1997, 49:290);(3) synthesis techniques, and (4) PCR based targeting. It will be understood by practitioners that these and other methods can be used in the invention, and that these methods may be applied to any number of parents and cut points. The recombination techniques of the invention include in vitro and in vivo recombination, as well as methods which combine both approaches, and firther, recombinants can be cloned and/or expressed by host cells according to known techniques.

Fragmentation and reassembly techniques utilize a restriction enzyme or set of restriction enzymes at specific concentrations to selectively cut biopolymer strands at identified locations. The choice and concentration of enzyme(s) are determined based upon the identified optimal crossover locations determined by the method of the invention. The method can be applied to homologous and non-homologous nucleic acid sequences. The resulting DNA fragments, produced by the restriction enzyme digest, can be reassembled by techniques known in the art, thereby creating hybrid parental DNA strands that can be used as templates for the production of proteins. The invention also encompasses the fragmentation and reassembly of amino acid sequences. The fragmentation and reassembly may be accomplished, for example, by the use of chemical methods or enzymes for homologous or non-homologous amino acid sequences.

Alternatively, the StEP method (Zhao et al., Nature Biotechnology 1997, 49:290) biases the creation of mutant hybrid proteins towards mutations at desired crossover locations. A set of DNA primers are synthesized to hybridize with equal probability to all parental strands at desired crossover recombination locations. The desired hybrid DNA sequence can be created by chemically synthesizing the desired DNA sequence or ligating synthesized fragments of the desired DNA sequence. One method is to synthesize fragments based upon optimal crossover locations from all the parents and randomly anneal the fragments to produce a recombinant library. A related method reduces the need to synthesize each full length parental gene by encompassing the use of overlap extension, a DNA polymerase, and partial synthesis of the genes of interest to create the full length gene of interest.

StEP Recombination.

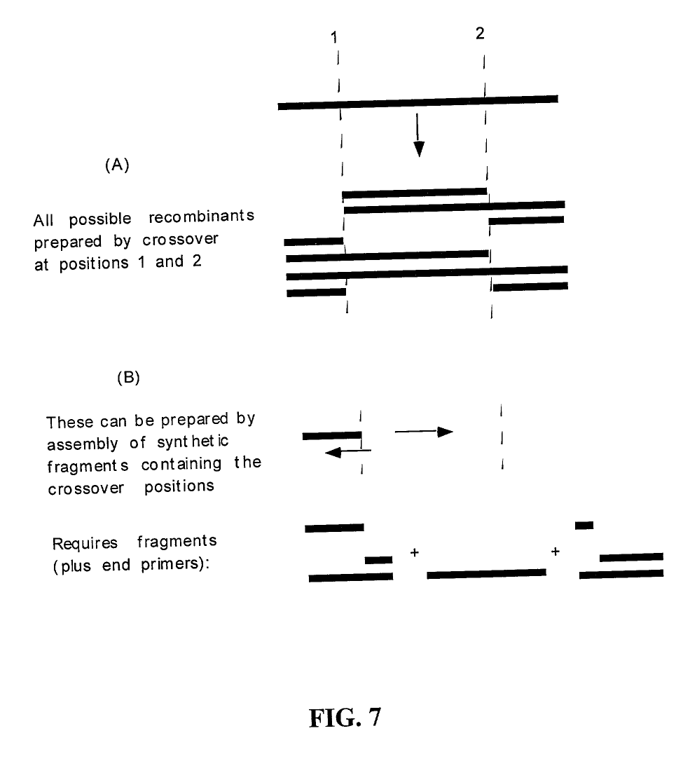

This approach is illustrated in FIG. 7 for two crossovers and two parental genes. Split pool synthesis can be used to minimize the synthesis burden. The method of Volkov et al., Nucl. Acids Res., 27:18 (1999) may be used. As shown in FIG. 7, a “grey” parent and a “black” parent are each cut at positions 1 and 2. Crossover recombination atthese cut points or crossover regions generates eight possible recombinants, including two that are identical to one of the parents. The remaining six recombinants have mutant sequences with contributions from each parent that cross over to a contribution from the other parent at one or both cut points. See, FIG. 7, part (A). Each of these recombinants can be made by assembly of synthetic fragments that contain the cut points or crossover locations, i.e. at least one of each pair of fragments to be joined contains residues from one or the other parent that extend past the cut point, as shown in FIG. 7, part (B). In this example, the terminal fragments have end primers that include a cut point, resulting in four possible fragments on the left, four on the right, and two (one from each parent) in the middle. These fragments can be reassembled in eight different sets of three, to produce each of the eight recombinants in FIG. 7, part (A).

In Vitro—In Vivo Recombination.

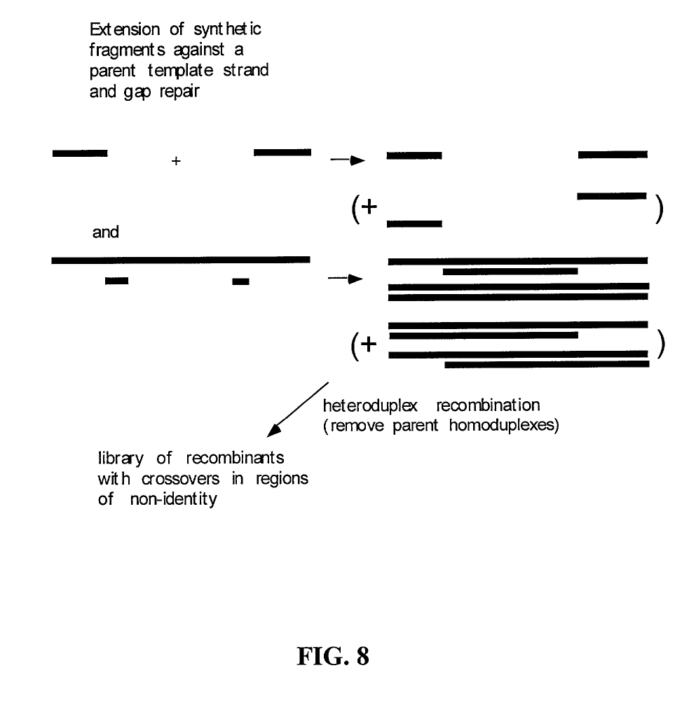

A hybrid in vitro-in vivo recombination method is outlined in FIG. 8. In FIG. 8, the method pertains to the shuffling of two parental genes. The method encompasses gene assembly using synthetic fragments and overlap extension with fragments followed by gap repair, which creates double stranded sequences containing mismatched regions. The mismatches are then repaired randomly in vivo when inserted into an appropriate host cell in the form of a heteroduplex plasmid. This method removes parental homoduplexes and results in a library of random crossovers near the mismatched sites for each of the two reactions. Further complexity (more crossovers) can be added easily by adding fragments corresponding to desired crossover points.

In FIG. 8, a “grey” parent and a “black” parent represent polymers (e.g. genes), to be cut and reassembled at two cut points. Synthetic fragments from each parent are extended at a cut point to correspond with the sequence of the other parent, by using the other parent as a template. For example, fragments derived from the black parent are extended at designated cut points with sequences from the grey parent, using the grey parent as a template. Fragments derived from the grey parent are likewise extended using the black parent as a template. This produces polymer duplexes, e.g. double strands of nucleic acid residues, representing the different possible combinations of fragments.

In the example of FIG. 8, with two cut points, two sets of four different duplexes are possible, for a total of eight duplexes. These represent the eight possible recombinations of sequences from the two parents by crossovers at the two cut points. Two of these duplexes are homoduplexes, meaning that the sequences of both polymers are identical to each other. They are also each identical to one of the parent polymers. The remaining six duplexes are heteroduplexes, meaning that the sequences of each polymer in the duplex pair are different. One member of each heteroduplex has a sequence identical to one of the parents. The other member of each heteroduplex pair is a crossover recombinant, with a sequence that crosses over from one parent to the other at one or more of the cut points. In this example, with two cut points, a crossover can occur at one or both cut points, resulting in two sets of three recombinant sequences that differ from parent sequences. As shown in FIG. 8, these six crossover recombinants are (black-grey-black), (grey-grey-black), (black-grey-grey), and the “reverse” set of recombinants (grey-black-grey), (black-black-grey), and (grey-black-black).

The duplexes produced by this method can be introduced to an appropriate host cell for heteroduplex recombination, which serves to remove the parent homoduplexes. The result is a library of crossover recombinants having sequences contributed by both parents.

It will be understood that this discussion and FIG. 8 is an illustration of a general technique that is applicable to the inventions. For example, more than two parents ad/or more than two cut points can be used.

PCR Amplification.

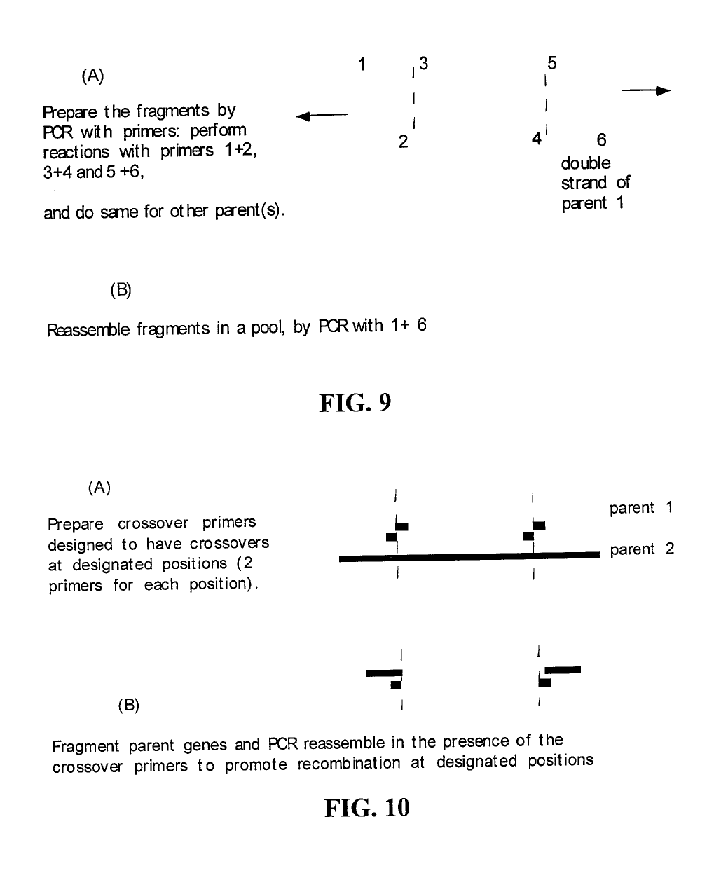

Another method is outlined in FIG. 9. Gene fragments for reassembly can be prepared by PCR with primers directed for crossovers. The primers can be designed such that a single primer will hybridize equally to all parent strands at the desired positions at crossover locations. The fragments prepared by these reactions are pooled and reassembled by PCR with flanking primers, e.g. 1+6 in the example. The resulting PCR products will have crossovers directed to locations of the primers.

As shown in FIG. 9, several sets of primers are made for each parent polymer. One set of primers corresponds to the terminal ends of the polymer. In this examples there is one primer for each of the 3′ and 5′ ends of a polynucleotide, designated 1 and 6 in FIG. 9. Each remaining set of primers corresponds to each cut point, and in this example there are two primers for each cut point. These are designated 2 and 3 for the cut point at the left, and 4 and 5 for the cut point at the right in FIG. 9. Similar sets of primers are prepared for each other parent. PCR amplification is performed using pairs of primers that flank adjacent regions of the polymer, e.g. primers 1 and 2, primers 3 and 4, and primers 5 and 6. All of the possible fragments from all of the parents are reassembled in a pool, using PCR reactions starting with primers 1 and 6.

Family Shuffling.

Another method is outlined in FIG. 10, which is a DNA shuffling method as described e.g. by the 1994 Stemmer references. The recombination is directed to specific sites utilizing “crossover” primers. The crossover primers are synthesized to contain crossover sequences and are used during the reassembly reaction. The concentration of the primer can be varied and can be much higher than that of the parental genes.

In this approach, sets of primer pairs are prepared. Each primer of each pair has sequences from two parents which span and include a designated crossover location. FIG. 10, part (A). The parent genes are fragmented, and fragments are reassembled in the presence of the primers using PCR amplification. The primers promote reassembly and amplification at the crossover locations they span, to produce complementary recombinants with sequences from more than one parent. FIG. 10, part (B). Two parents and two cut points are shown in this example, but more may be used. In the figure, a partially reassembled sequence for one recombinant is shown, with terminal sequences coming from one patent (black) and the middle or intervening sequences coming from another parent (grey).

The methods described above and illustrated by FIGS. 7-10 are novel methods for targeting optimal crossover locations, in particular based on the techniques calculations described herein, e.g. in Sec. 5.4 above.

Screening Hybrids With Protected Schema

According to the invention, crossovers at locations that minimally disrupt coupling interactions with other residues are more likely to lead to functional proteins. By focusing the crossovers in a directed evolution experiment to residues having crossover locations that minimize the disruption of coupling interactions or domains, the number of sequences that must be tested or screened is considerably reduced.

Referring specifically to embodiments where the parent sequence is a protein or other polypeptide sequence, the parent sequence (and mutants thereof) may be expressed in facile gene expression systems to obtain libraries of mutant proteins. Any source of nucleic acid in purified form can be utilized as the starting nucleic acid. Thus, the process may employ DNA or RNA, including messenger RNA. The DNA or RNA may be either single or double stranded. In addition, DNA-RNA hybrids which contain one strand of each may be utilized. The nucleic acid sequence may also be of various lengths depending on the size of the sequence to be mutated. Preferably, the specific nucleic acid sequence is from 50 to 50,000 base pairs. It is contemplated that entire vectors containing the nucleic acid encoding the protein of interest may be used in these methods.

Once the evolved polynucleotide molecules are generated they can be cloned into a suitable vector selected by the skilled artisan according to methods well known in the art. If a mixed population of the specific nucleic acid sequence is cloned into a vector it can be clonally amplified by inserting each vector into a host cell and allowing the host cell to amplify the vector. The mixed population may be tested to identify the desired recombinant nucleic acid fragment. The method of selection will depend on the DNA fragment desired. For example, in this invention a DNA fragment which encodes for a protein with improved properties can be determined by tests for functional activity and/or stability of the protein. Such tests are well known in the art.

Using the methods of directed evolution, the invention provides a novel means for producing functional, and soluble proteins with improved activity toward one or more substrates. The mutants can be expressed in conventional or facile expression systems such as E. coli. Conventional tests can be used to determine whether a protein of interest produced from an expression system has improved expression, folding and/or functional properties. For example, to determine whether a polynucleotide subjected to directed evolution and expressed in a foreign host cell produces a protein with improved activity, one skilled in the art can perform experiments designed to test the functional activity of the protein. Briefly, the evolved protein can be rapidly screened, and is readily isolated and purified from the expression system or media if secreted. It can then be subjected to assays designed to test functional activity of the particular protein.

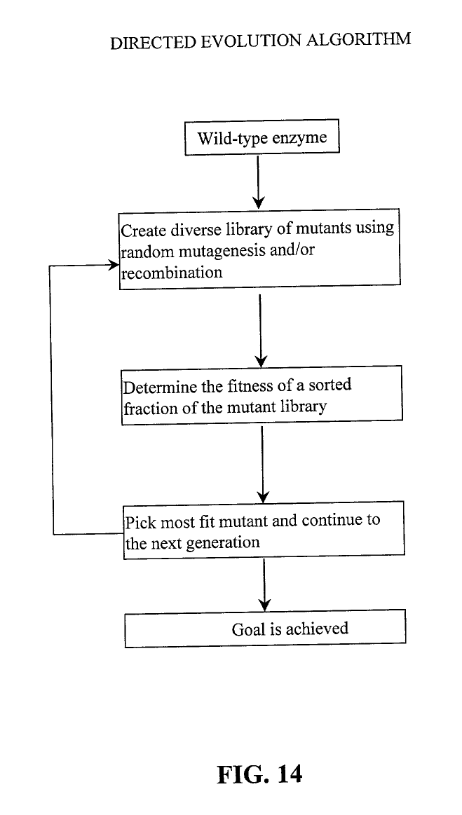

A flow chart of an exemplary directed evolution algorithm is illustrated in FIG. 14. A library of mutants can be made by any of the methods described herein. The library can be sorted or restricted using the computational methods of the invention to identify the most promising subset of “fit” mutants. These can be screened to pick the most fit mutant. This process can be repeated in successive generations, until no further changes are observed, a set goal is achieved, or the process is ended at any desired step.

5.8 Implementation Systems and Methods

Computer System.

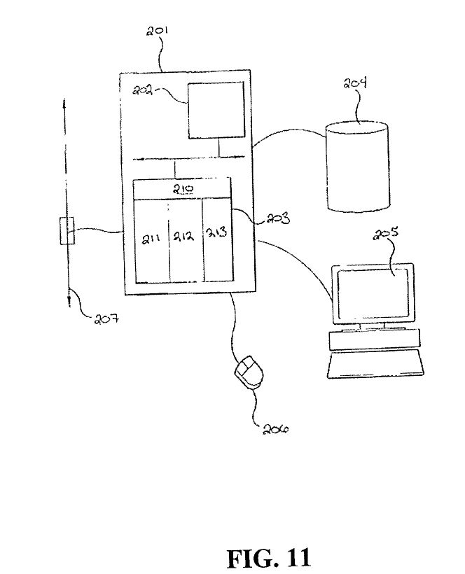

The analytical methods described in the previous subsections may preferably be implemented by the use of one or more computer systems, such as those described herein. Accordingly, FIG. 11 schematically illustrates an exemplary computer system suitable for implementation of the analytical methods of this invention. Computer 201 is illustrated here as comprising internal components linked to external components. However, a skilled artisan will readily appreciate that one or more of the components described herein as “internal” may, in alternative embodiments, be external. Likewise, one or more of the “external” components described here may also be internal. The internal components of this computer system include processor element 202 interconnected with a main memory 203. For example, in one preferred embodiment computer system 201 may be a Silicon Graphics R10000 Processor running at 195 MHz or greater and with 2 gigabytes or more of physical memory. In another, less preferable, exemplary embodiment, computer system 201 may be an Intel Pentium based processor of 150 MHz or greater clock rate and the 32 megabytes or more of main memory.

The external components may include a mass storage 204. This mass storage may be one or more hard disks which are typically packaged together with the processor and memory. Such hard disks are typically of at least 1 gigabyte storage capacity, and more preferably have at least 5 gigabytes or at least 10 gigabtyes of storage capacity. The mass storage may also comprise, for example, a removable medium such as, a CD-ROM drive, a DVD drive, a floppy disk drive (including a Zip™ drive), or a DAT drive or other. Other external components include a user interface device 205, which can be, for example, a monitor and a keyboard. In preferred embodiments the user interface is also coupled with a pointing device 206 which may be, for example, a “mouse” or other graphical input device (not illustrated). Typically, computer system 201 is also linked to a network link 207, which can be part of an Ethernet or other link to one or more other, local computer systems (e.g., as part of a local area network or LAN), or the network link may be a link to a wide area communication network (WAN) such as the Internet. This network link allows computer system 201 to communicate with one or more other computer systems.

Typically, one or more software components are loaded into main memory 203 during operation of computer system 201. These software components may include both components that are standard in the art and special to the invention, and the components collectively cause the computer system to function according to the analytical methods of the invention. Typically, the software components are stored on mass storage 204 (e.g., on a hard drive or on removable storage media such as on one or more CD-ROMs, RW-CDs, DVDs, floppy disks or DATs). Software component 210 represents an operating system, which is responsible for managing computer system 201 and its network interconnections. This operating is typically an operating system routinely used in the art and may be, for example, a UNIX operating system or, less preferably, a member of the Microsoft Windows™ family of operating systems (for example, Windows 2000, Windows Me, Windows 98, Windows 95 or Windows NT) or a Macintosh operating system.

Software component 211 represents common languages and functions conveniently present in the system to assist programs implementing the methods specific to the invention. Languages that may be used include, for example, FORTRAN, C, C++ and less preferably JAVA.

The analytical methods of the invention may also be programmed in mathematical software packages which allow symbolic entry of equations and high-level specification of processing, including algorithms to be used, thereby freeing a user of the need to procedurally program individual equations and algorithms. Examples of such packages include Matlab from Mathworks (Natick, Mass.), Mathematica from Wolfram Research (Champaign, Ill.) and S-Plus from Math Soft (Seattle, Wash.). Accordingly, software component 212 represents the analytic methods of the invention as programmed in a procedural language or symbolic package.

The memory 203 may, optionally, further comprise software components 213 which cause the processor to calculate or determine a three-dimensional structure for a macromolecule and, in particular, for a given polymer sequence such as a protein or nucleic acid sequence. Such programs are well known in the art, and numerous software packages are available. This software includes Swiss-PdbViewer (Glaxo Wellcome Experimental Research); Biograf (Molecular Simulations, Inc); 0 (generally used for crystallography); Explorer (MSI); Quenta, CHARMM; and Sybil (Tripos). The memory may also comprise one or more other software components, such as one or more other files representing, e.g., one or more sequences of polymer residues including, for example, a parent sequence and/or other sequences (for example, mutant sequences). The memory 203 may also comprise one or more files representing the three-dimensional structures of one or more sequences, including a file representing the three-dimensional structure of a parent sequence, such as a parent protein or nucleic acid.

Computer Program Products.

The invention also provides computer program products which can be used, e.g., to program or configure a computer system for implementation of analytical methods of the invention. A computer program product of the invention comprises a computer readable medium such as one or more compact disks (i.e., one or more “CDs”, which may be CD-ROMs or a RW-CDs), one or more DVDs, one or more floppy disks (including, for example, one or more ZIP™ disks) or one or more DATs to name a few. The computer readable medium has encoded thereon, in computer readable form, one or more of the software components 212 (FIG. 11) that, when loaded into memory 203 of a computer system 201, cause the computer system to implement analytic methods of the invention. The computer readable medium may also have other software components encoded thereon in computer readable form. Such other software components may include, for example, functional languages 211 or an operating system 210. The other software components may also include one or more files or databases including, for example, files or databases representing one or more polymer sequences (e.g. protein or nucleic acid sequences) and/or files or databases representing one or more three-dimensional structures for particular polymer sequences (e.g., three-dimensional structures for proteins and nucleic acids.

System Implementation.

In an exemplary implementation, to practice the methods of the invention a parent sequence may first be loaded into the computer system 201 (FIG. 11). For example, the parent sequence may be directly entered by a user from monitor and keyboard 205 and by directly typing a sequence of code of symbols representing different residues (e.g., different amino acid or nucleotide residues). Alternatively, a user may specify parent sequences, e.g., by selecting a sequence from a menu of candidate sequences presented on the monitor or by entering an accession number for a sequence in a database (for example, the GenBank or SWISPROT database) and the computer system may access the selected parent sequence from the database, e.g., by accessing a database in memory 203 or by accessing the sequence from a database over the network connection, e.g., over the internet.

The programs may then cause the computer system to obtain a three-dimensional structure of the parent sequence. For example, the three-dimensional structure for the parent sequence may also be accessed from a file (for example, a database of structures) in the memory 203 or mass storage 204. Alternatively, the three-dimensional structure may also be retrieved through the computer network (e.g., over the network) from a database of structures such as the PDB database. In yet other embodiments, the software components may, themselves, calculate a three-dimensional structure using the molecular modeling software components. Such software components may calculate or determine a three-dimensional structure, e.g., ab initio or may use empirical or experimental data such as X-ray crystallography or NMR data that may also be entered by a user of loaded into the memory 203 (e.g., from one or more files on the mass storage 204 or over the computer network 207). The software components may further cause the computer system to calculate a conformational energy for the parent sequence using the three-dimensional structure.

Finally, the software components of the computer system, when loaded into memory 203, preferably also cause the computer system to determine a coupling matrix or, in the alternative, a parameter related to or correlating with coupling interactions according to the methods described herein. For example, the software components may cause the computer system to generate one or more mutant sequences of the parent and, using the conformation determined or obtained for the parent sequence, determine coupling interactions and well as disrupted coupling interactions.

Upon implementing these analytic methods, the computer system preferably then outputs, e.g., the coupling constants of the parent sequence or the disruption profile of the mutant pool. For instance, the coupling interactions may be output to the monitor, printed on a printer (not shown) and/or written on mass storage 204. In preferred embodiments, the software components may also cause the computer system to select and identify one or more particular crossover locations in the parent sequence for mutation, e.g., in a directed evolution experiment. For example, the computer system may identify residues of the parent sequence having as crossover locations that minimally disrupt coupling interactions. These residues could be identified, for a user, as ones which, if mutated, are most likely to improve properties of the polymer in a directed evolution experiment while retaining function.

Alternative systems and methods for implementing the analytic methods of this invention are also intended to be comprehended within the accompanying claims. In particular, the accompanying claims are intended to include the alternative program structures for implementing the methods of this invention that will be readily apparent to those skilled in the relevant art(s).

### 6. EXAMPLES

The present invention is also described by means of particular examples. However, the use of such examples anywhere in the specification is illustrative only and in no way limits the scope and meaning of the invention or of any exemplified term. Likewise, the invention is not limited to any particular preferred embodiments described herein. Indeed, many modifications and variations of the invention will be apparent to those skilled in the art upon reading this specification and can be made without departing form its spirit and scope. The invention is therefore to be limited only by the terms of the appended claims along with the full scope of equivalents to which the claims are entitled.

6.1 Computational Determination of Structural Schema

Structural schema of a biopolymer, e.g. a gene or protein, can be identified, and crossover disruption profiles of identified schema can be calculated. These calculations can be used to predict optimal crossover locations and resulting recombinant offspring that are more likely to be stable, and exhibit new or improved properties. Schema disruption profiles can be based on energy or distance calculations, or both. A preferred method, for its relative computational efficiency, is based on interatomic distances.

Crossover Disruption Based on Interatomic Distances

Computing the distances between atoms, rather than a detailed energy calculation, can significantly accelerate the calculation of coupling interactions between residues. To perform this calculation, a structure file (such as a Protein Databank PDB or Biograf BGF file) is read that contains the coordinates for each atom of this structure. The distances between all atoms are calculated with the equation,

dᵢⱼ=\sqrt{(xᵢ−xⱼ)²+(yᵢ−yⱼ)²+(zᵢ−zⱼ)²} (1)

where dᵢⱼ is the distance between atoms i and j, and (xᵢ,yᵢ,zᵢ) are the three-dimensional coordinates of atom i. Two residues are considered coupled if any of their atoms (both side chain and main chain, excluding hydrogens) are within a cutoff distance d_{c}. The parameter d_{c} is set such that the average number of coupling interactions per residue is between 4 and 12. The preferred value for d_{c} is 4.0 angstroms, corresponding to approximately 7-8 interactions per residues. A two-dimensional coupling matrix c is used to keep track of the coupled residues. An element of this matrix cᵢⱼ is equal to one if residues i and j are within distance d_{c} and is zero otherwise.

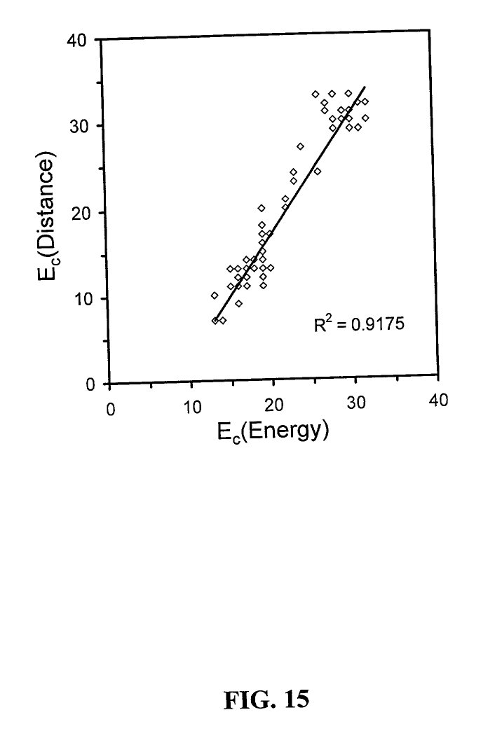

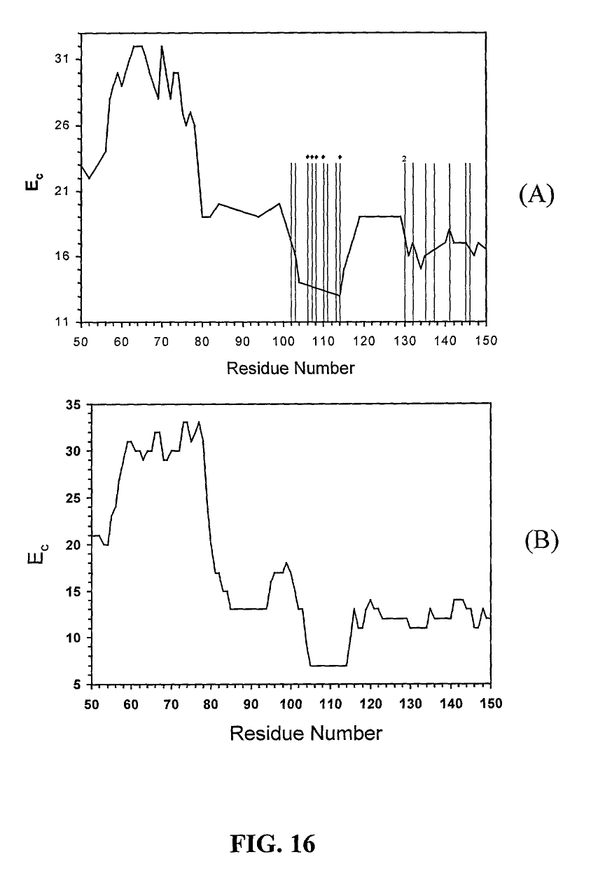

Despite the beneficial reduction in computation complexity, the disruption results based on distance rather than energy calculations are not significantly altered. FIG. 15 compares the calculation of the single-cut-point crossover disruption for transformylase based on the distance (top) and energy (bottom) definitions of coupling. The qualitative shape of both plots is similar and a quantitative comparison of both measures (yields R²−0.91. FIG. 16(A) shows a plot based on energy, and FIG. 16(B) shows a plot based on distance. Due to the significant improvement in calculation time, the distance-based definition of coupling is a preferred mode for the disruption calculations.

The crossover disruption of a fragment can then be calculated using the equation

$${E}_{c , α} = {\sum }_{i ∉ α}^{{N}_{T}} {\sum }_{j ∈ α}^{{N}_{d}} {c}_{ij} {P}_{i} {P}_{j} \qquad ( 2 )$$

where iα indicates the summation over the residues not in the fragment α and iεα indicates the summation over the residues in fragment α. N_{T} is the total number of residues, N_{d} is the number of residues in fragment α, cᵢⱼ is the coupling matrix and Pᵢ is the probability that two parents have different amino acid identities at residue i. The probabilities Pᵢ and Pⱼ are determined by examining a sequence alignment of the parents and counting the number of times that the parents share an amino acid identity at that residue according to:

$${P}_{i} = ( \begin{matrix}{n}_{p} \\ 2\end{matrix} ) {\sum }_{j = 1}^{{n}_{p}} {\sum }_{k > i}^{{n}_{p}} {s}_{jk} \qquad ( 3 )$$

where s^{jk}=1 if parent j and k have the same amino acid at residue i, and nₚ is the total number of parents. When the sequences of the parents are unknown, or if it is desirable to count disruption at positions where the amino acid identities are identical, Pᵢ can be set to unity for all i. Further, Equation (3) could be modified to reflect physio-chemical similarities (such as charge, hydrophobicity, size) between amino acids, thus weighing crossover disruption more heavily when comparing dissimilar amino acids.

Disrupting Folding Domains

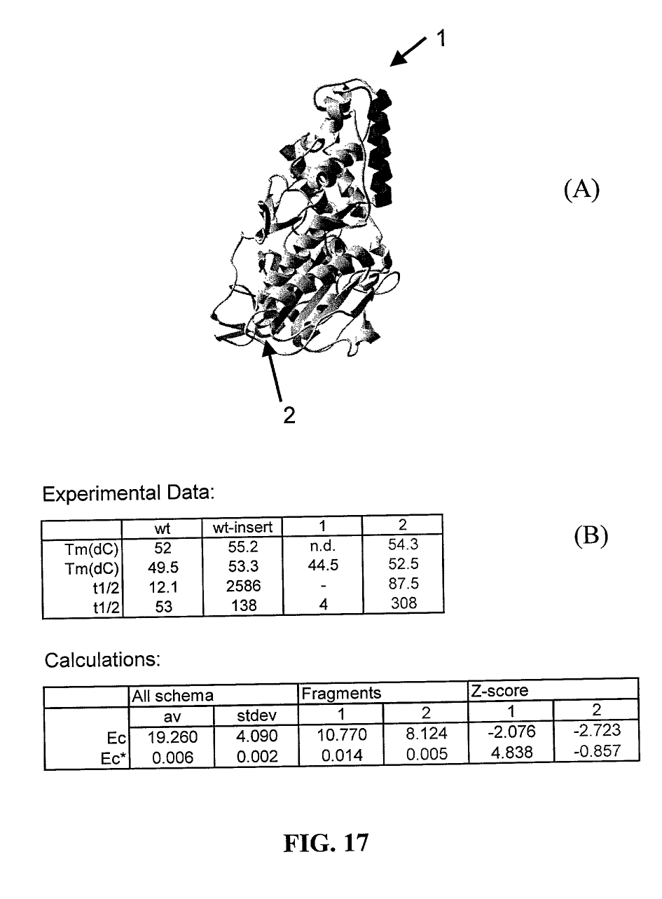

A data set generated by the experimental engineered shuffling of a thermostable phytase with a mesostable phytase yields further insight into the disruption caused by domain substitution. Jermutus, et al., Structure-based chimeric enzymes as an alternative to directed enzyme evolution: phytase as a test case, J. Biotech., 85: 15-24 (2001). In this experiment, two chimeric proteins were created by extracting small domains (1, 2) from the thermophilic (A. niger) phytase and inserting them into the less-stable (A. terreus) phytase. FIG. 17(A) One chimera (HyA) was created by inserting a surface helix (residues 66-82), FIG. 17(1). The second chimera (HyB) was created by inserting a buried beta-strand (residues 48-58), (FIG. 17(1). HyA2 was stabilized when compared to the A. terreus wild-type and HyB1 was significantly destabilized.

This is shown by the comparison of melting temperatures (Tₘ) in degrees C for HyA (mutant 2) and HyB (mutant 1) reported in the Experimental Data of FIG. 17. The melting temperature of HyA (2) is higher than for HyB (1), meaning it is relatively more stable (more energy, i.e. a higher temperature, is needed to cause unfolding. Similarly, the temperatures at which 50% of the HyA and HyB proteins are unfolded (t_{½}) show that HyA is more stable and HyB is less stable. FIG. 17 also shows comparison data for thermodynamic properties of the wild type A. terreus phytase enzyme (wt) and the wild type thermophilic A. niger phytase (wt-insert).

To determine the disruptiveness of the two domains, the crossover disruption was calculated for each domain insertion and statistically compared to the disruptiveness of all fragments in phytase. The crossover disruption E_{c} of the HyA mutant is 8.12 and HyB is 10.77 (FIG. 17, Calculations). While HyA is less disruptive, both compare well to the average crossover disruption 19.26 (standard deviation 4.09), calculated by determining the disruptiveness of all possible fragments. To emphasize this trend, the Z-score was calculated for each chimera, where the Z-score Zᵢ of fragment i is defined as:

$${Z}_{i} = \frac{{E}_{c , i} - < {E}_{c} >}{σ ( {E}_{c} )} \qquad ( 4 )$$

where E_{ci} is the crossover disruption of fragment i, <E_{c}> is the average crossover disruption of all fragments, and s(E_{c}) is the standard deviation of the crossover disruption of all fragments. The Z-score of HyA is −2.72 and HyB is −2.08 (FIG. 17), indicating that while HyA is predicted to be a more acceptable substitution than HyB, both have a very low disruption when compared to the average.

Both chimeras have relatively low crossover disruption values because they are both small fragments. Normalizing the crossover disruption measure by the number of residues in the fragment N_{d} and the total number of residues N_{T} overcomes this effect; given by:

$${E}_{c}^{*} = \frac{{E}_{c}}{{N}_{d} ( {N}_{T} - {N}_{d} )} , \qquad ( 5 )$$

where E_{c}* is the normalized disruption measure. Other possibilities include normalizing the crossover disruption by the number of residues in the domain alone,

$${E}_{c}^{*} = \frac{{E}_{c}}{{N}_{d}} \qquad ( 6 )$$

and normalizing the square-root of the crossover disruption by the number of residues in the domain,

$${E}_{c}^{*} = \frac{\sqrt{{E}_{c}}}{{N}_{d}} . \qquad ( 7 )$$

When equation 3 is used to calculate the disruptiveness of the substitutions into phytase, HyA has a disruption of 0.005 and HyB has a disruption of 0.014 (FIG. 17). The average disruption for all possible fragments is 0.006 (standard deviation is 0.002). The Z-scores of the HyA substitution is −0.86 and the HyB substitution is 4.838, indicating that by these measures, the HyB substitution is far more likely to be destabilizing, as was found experimentally (Jermutus et. al, 2001). In general, Equation (6) is the preferred mode of the calculation due to the lack of dependence on the total number of residues.

The normalized value for crossover disruption (Equation 6) can be used to determine the compatibility of isolated fragments when substituted into the remaining structure. As an example, the crossover disruption was calculated for fragments that appeared in the DNA shuffling experiment with beta-lactamase (Crameri, 1998). Each fragment independently exhibits a low crossover disruption. When a list of possible fragments is known before an experiment, this type of calculation could be used to computationally separate a subgroup of fragments that are more likely to produce folded chimeras, based on their disruptiveness of the structure. This approach could be applied to methods of “exon shuffling,” whereby parent genes are fragmented and recombined at crossover points based on their natural intron-exon structure on the gene level. Kolkman & Stemmer, Nature Biotechnology, 19: 423-428 (2000). The computational method is able to determine the sets of exons that are least likely to be disruptive when substituted into the structure.

Recombination can cause disruption on two levels of the hierarchical protein folding process. First, the internal energy can be disturbed by the substitution of a parental fragment that disturbs the interactions that stabilize the structure (the crossover disruption). Second, if a fragment causes a highly concentrated region of crossover disruption, then this region is unlikely to fold. Even if the remainder of the structure has a low internal energy (few broken coupling interactions), the locally misfolded region would be severely destabilizing. Combined, the phytase and beta-lactamase data sets support this view of disruption. In both experiments, crossovers that distributed the disruption throughout the gene, rather than localized regions of high crossover disruption generated stable chimeras. Practically, this implies that it is better to have a large absolute crossover disruption (large total E_{c}) that is well distributed across the gene (low E_{c}* for all the fragments), than have a small absolute crossover disruption (low total E_{c}) that is very localized (large E_{c}* for one fragment).

Calculating Compact Units of Structure

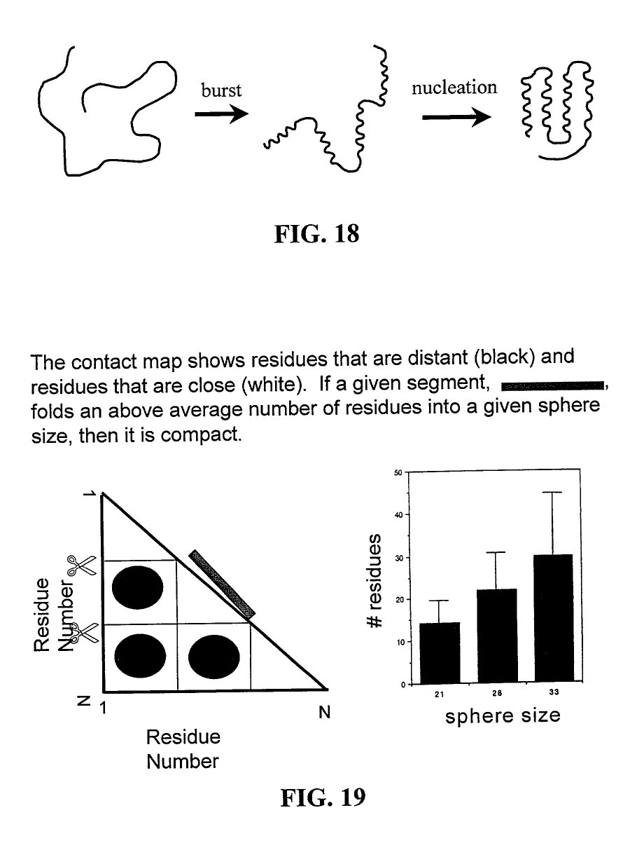

The current view of protein folding is that the process is hierarchical. First, a very fast “burst” phase occurs where the unfolded polypeptide rapidly collapses into highly- compact units, such as alpha-helixes. Next, the substructures condense into the tertiary arrangement of the native structure (FIG. 18). The experimental observation that folding is hierarchal has led to the “building block” theory that proteins have subunits that fold and then assist higher-level rearrangements. Tsai, C -J., et al., Anatomy of protein structures: visualizing how a one-dimensional protein folds into a three-dimensional shape, Proc. Natl. Acad. Sci. USA, 97: 12038-12043 (2000). According to the invention, crossovers that do not disrupt these building blocks will be more likely to lead to functional chimeras.

A useful tool to visualize local units of condensed structure (“building blocks”) is the contact map. Rossman, M. G, & Lilj as, A., J. Molec. Biol., 85: 177-181. The contact map is constructed by measuring the distance between all alpha-carbons in the three-dimensional structure (Equation 1) and then generating a two-dimensional matrix where residues that are within a cutoff distance dᵣₒₛₛ are marked as white whereas residues that lie outside this cutoff distance are marked as black. Domains that occur on the level of the one-dimensional polypeptide chain can be identified as triangles that can be drawn on the diagonal that do not contain any black regions (FIG. 19). Effectively, this identifies fragments of the structure that fold into a sphere of diameter dᵣₒₛₛ.

Several algorithms have been proposed to divide the contact map into regions, thus identifying domains in the structure. See e.g., De Souza, et al., Intron positions correlate with module boundaries in ancient proteins, Proc. Natl. Acad. Sci. USA, 93: 14632-14636 (1996); Gilbert, et al., Origin of Genes, Proc. Natl. Acad. Sci. USA, 94: 7698-7703 (1997); Go, M., Correlation of DNA exonic regions with protein structural units in haemoglobin, Nature, 291: 90-92 (1981); Go, M., Modular structural units, exons, and function in chicken lysozyme, Proc. Natl. Acad. Sci. USA, 80: 1964-1968 (1983).

Go originally proposed that lines should be drawn that cross through the largest white regions with the intent to separate the black regions. This fragments the structure into domains in a way that minimizes the interaction between the domains. While this algorithm was crudely successful in demonstrating the correlation between exons and subdomains, it often fails on complicated structures that do not have an obvious domain structure. Measuring the number of interactions at each site can quantitate this algorithm,

$${R}_{i} = {\sum }_{i = 1}^{N} {Δ}_{ij} \qquad ( 8 )$$

where Δᵢⱼ=0 if residues i and j are closer than dᵣₒₛₛ and Δᵢⱼ=1 if residues i and j are farther than dᵣₒₛₛ. According to Go, residues that minimize Rᵢ are more likely to be regions between domains.

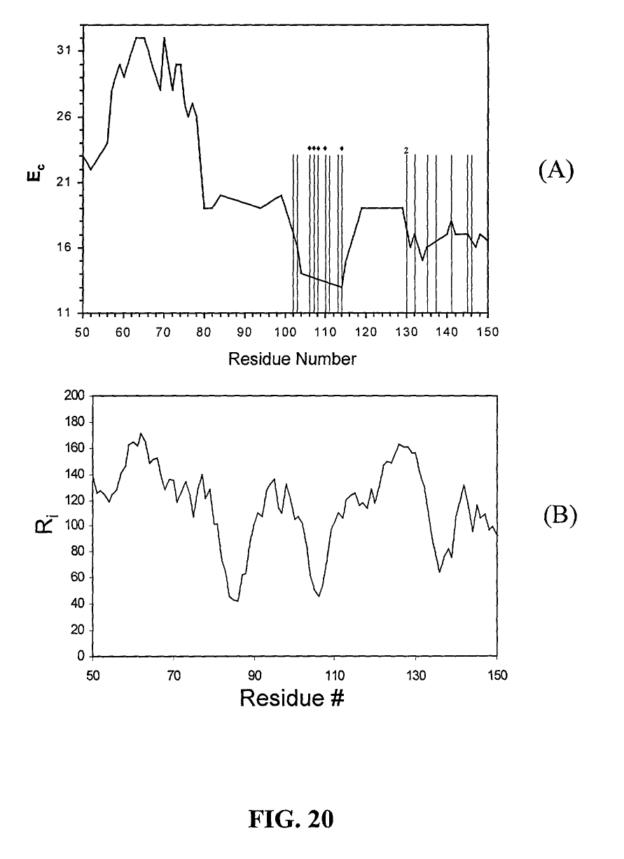

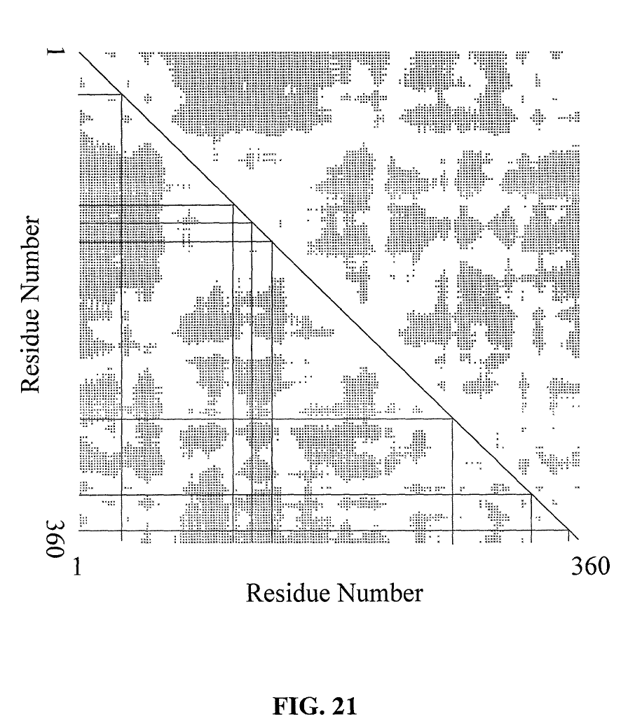

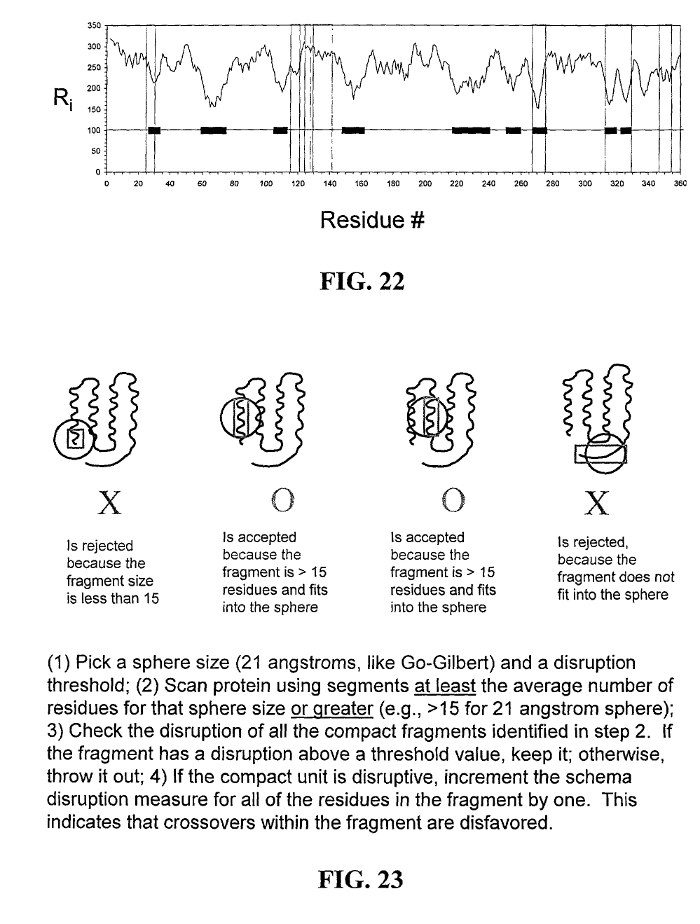

In this example a plot of Rᵢ for transformylase was generated. FIG. 20(B). This algorithm predicts that there are three domain-forming regions in the protein structure (three valleys), whereas two were sampled in the in vitro recombination experiment (FIG. 20A). This indicates that, while crossovers in this region could form a domain, too many coupling interactions are disrupted between the fragments, thus leading to destabilized structures. Further, a calculated contact map (FIG. 21) and a plot of Rᵢ (FIG. 22) for beta-lactamase show that, while some crossovers occurred in regions that are predicted to separate domains, this algorithm was relatively weak for predicting crossover locations. Other domain-separating algorithms based on analyzing the contact map have been proposed, but are not reliably consistent when analyzing the locations of crossovers in recombination experiments (De Souza et al, 1996; Gilbert et al, 1997).

SCHEMA: Schema-based Hybrid Protein Optimization

The present method identifies domains (“building blocks”) in proteins based on analyzing the contact map to optimize recombinants based on schema. FIG. 23. This algorithm is based on searching the protein structure for regions that are compact, based on comparing the length of a fragment with the size of the sphere into which the fragment folds. Gilbert and co-workers found that, for a domain diameter of dᵣₒₛₛ=21 angstroms, the average fragment that can fold into this sphere is 15 residues long with a standard deviation of 5 residues (De Souza et al, 1996). In other words, if a fragment of 20 residues folds such that all the residues are within a sphere of 21 angstroms, then this fragment can be considered as being highly compact. Further, if a fragment of 15 residues folds into a sphere of 21 angstrom, then the compactness of this unit is statistically average. This observation is utilized here by choosing a minimum fragment length nₘᵢₙ that, if a fragment of this size or greater is folded into a sphere of diameter dᵣₒₛₛ, then this fragment is considered to be compact. Schema theory predicts that these compact units (“building blocks”) should not be disrupted by crossovers.

To determine the regions that are compact, the entire protein structure is scanned with fragments of size nₘᵢₙ and greater (FIG. 23). Each fragment is checked for whether it can fold into a sphere of dᵣₒₛₛ by inspecting the contact map for any regions of black (residues that are separated by more than dᵣₒₛₛ angstroms) in the triangle that defines the fragment. If there is no back in the triangle, then a compact unit is defined and crossovers are disfavored along the fragment because this would disrupt a structural building block. To demark this, a schema disruption profile is defined where higher values indicate a more disruptive event. The profile is defined by

$${S}_{i} = {\sum }_{j = 1}^{{N}_{T}} {\sum }_{{k = j + {n}_{min}}_{i ∈ ( j , k )}}^{{N}_{T}} δ ( {\sum }_{m = j}^{k} {\sum }_{n = j}^{k} {CM}_{mn} ) \qquad ( 9 )$$

where CMₘₙ is the element of the contact matrix corresponding to residues m and n, and d(f) is a function that is equal to 1 iff=0 and equal to 0 iff>0. Effectively, Equation (9) counts the number of times that residue i is involved in a compact unit. A residue that has a large Sᵢ value is involved in a more compact unit than a residue that has a low Sᵢ value.

Making crossovers in building blocks that interact with structure is more disruptive than making crossovers in building blocks that are isolated from the remainder of the structure. Following this idea, the algorithm combines the crossover disruption measure (based on the disturbance of coupling interactions) with the domain-based disruption measure to identify the compact units that are nucleating in folding (FIG. 23). To do this, we add a term to Equation 9 such that fragments that fold into a compact unit, but are not interacting with the remainder of the structure are not counted in the schema disruption profile. The modified equation is

$${S}_{i} = {\sum }_{j = 1}^{{N}_{T}} {\sum }_{{k = j + {n}_{min}}_{i ∈ ( j , k )}}^{{N}_{T}} δ ( {\sum }_{m = j}^{k} {\sum }_{n = j}^{k} {CM}_{mn} ) g ( {E}_{c}^{*} , {E}_{c , thresh} ) \qquad ( 10 )$$

where the function g(x,y) is equal to 1 if x>y and 0, otherwise. The schema disruption profile generated by Equation (10) identifies the regions of the protein that are involved in a compact unit that significantly contributes to the stability of the protein (many coupling interactions). If a crossover occurs in these regions, then it is more likely to have a destabilizing effect on the structure.

In Vitro Recombination Results: Beta-lactamase, Transformylase, P450

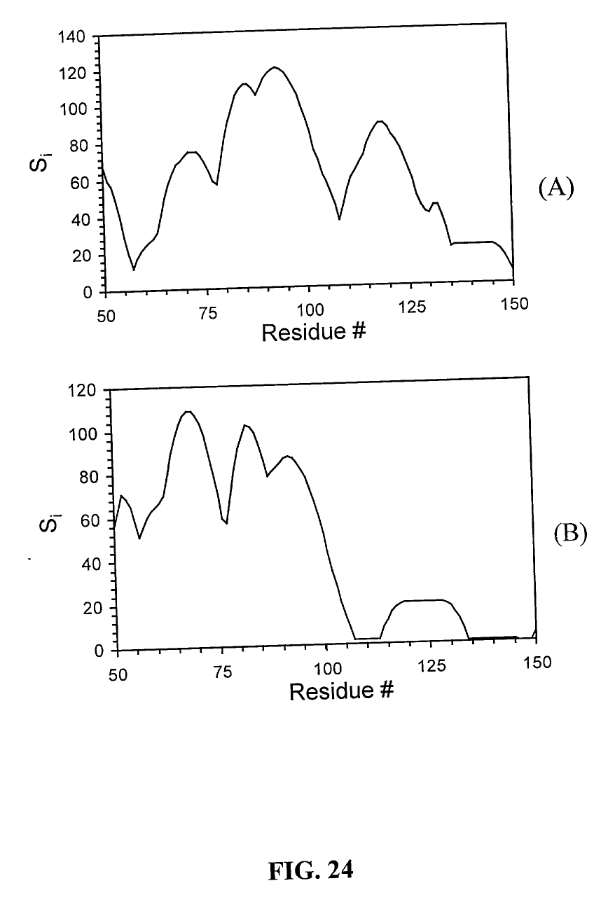

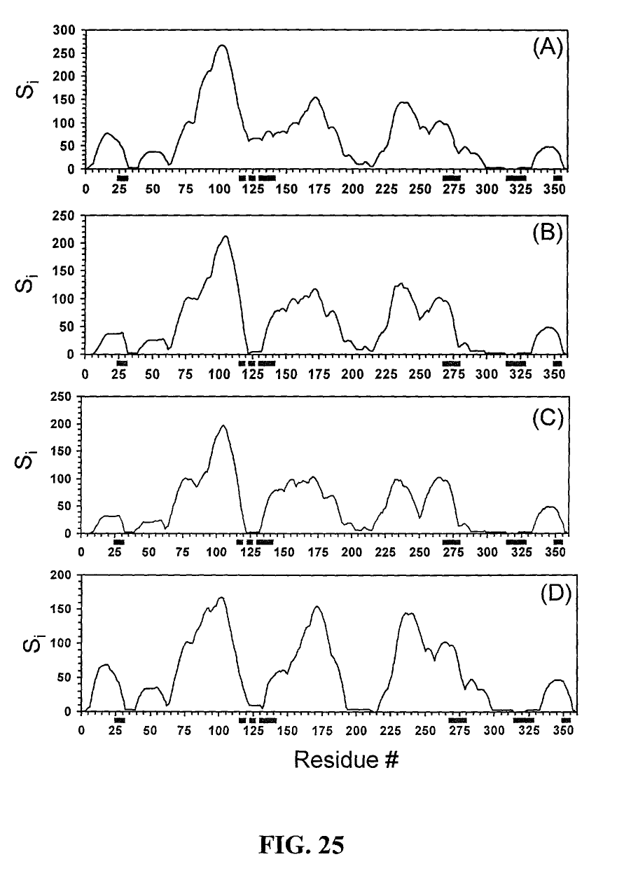

The results of the SCHEMA calculation on the transformylase and beta-lactamase data sets using the schema-based algorithm are shown in FIGS. 24 and 25, respectively. The algorithm rapidly locates the regions in which crossovers are disruptive. The advantages of the schema calculation over the alignment-based algorithm are threefold. First, the calculation is deterministic and does not rely on sampling or the method of computational hybridization that is used to reconstruct chimeric genes in silico. Second, the SCHEMA calculation only requires the structure file and does not rely on the accuracy of an alignment algorithm. Finally, the minima in the schema disruption profile are the optimal cut points, whereas the maxima in the stochastic algorithm are the statistically most likely cut points.

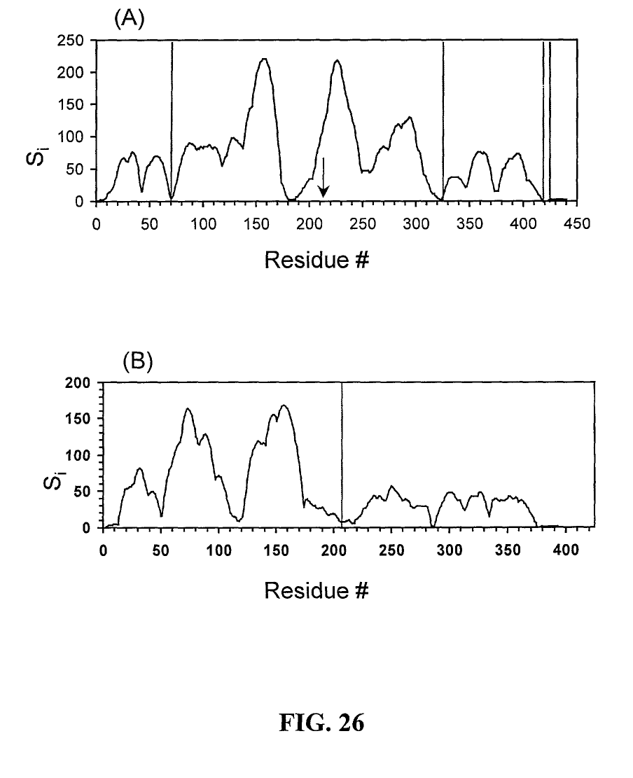

The algorithm predictions were compared with an in vitro evolution experiment that recombined low-sequence identity (25%) P450scc and P450c27 genes. Pikuleva, et al., Studies of distant members of the P450 superfamily (450scc and 450c27) by random chimeragenesis, Archives of Biochem. And Biophys., 334: 183-192 (1996). In this experiment, several chimeras were generated that folded into the native structure. While the structures of scc and c27 are unknown, the structure of a mammalian P450 (2C5) was recently solved. The schema disruption profile for the 2C5 structure was calculated (FIG. 26A) and was compared to the crossovers that resulted in folded chimeric sequences. The equivalent locations for the crossovers were determined by running a BLAST alignment of the local region around the crossovers as reported by Waterman and co-workers. Pikuleva, Bjorkhem & Waterman, M. R., Archives of Biochem. And Biophys., 334: 183-192 (1996). These crossovers are in regions that are predicted to be the least disruptive. In another experiment, a bacterial P450cam and human P450 2C9 were recombined at a single cut point. Shimoji, M., et al., Design of a novel P450: a functional bacterial-human cytochrome P450 chimera, Biochemistry, 37: 8848-8852 (1998). The chimera that resulted from this rationally-designed cut point folded successfully. The crossover occurred at a location that is minimally disruptive in the P450cam structure and near a minimum in the 2C5 structure (FIG. 26B. Together, these recombination experiments lend further support to the disruption calculations. See also, Hennecke, et al., Random circular permutation of DsbA reveals segments that are essential for protein folding and stability, J. Mol. Biol., 286: 1197-1215 (1999); Pachenko, et al., Foldons, protein structural modules, and exons, Proc. Natl. Acad. Sci. USA, 93: 2008-2013 (1996).

The optimal parents for experimental methods that restrict the fragmentation (such as DNA shuffling, restriction enzyme approaches, exon shuffling) can be determined by analyzing the schema disruption profile. The parents, exons, or restriction enzymes can be chosen such that the cut points occur at locations in the gene that minimize the schema disruption.

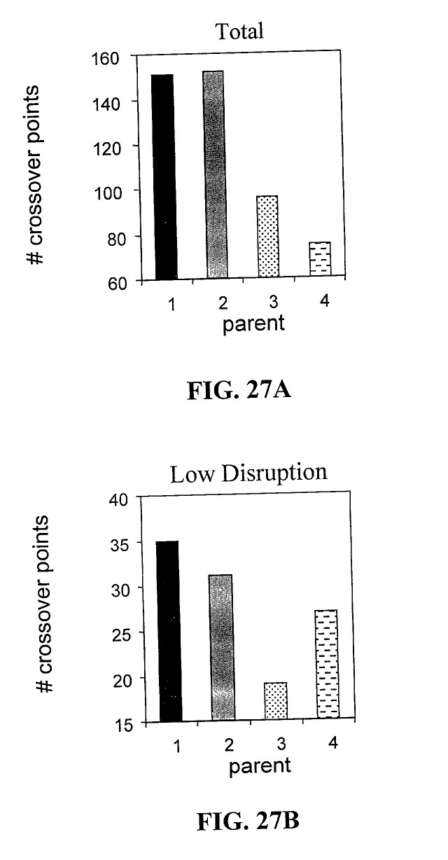

FIG. 27A shows the total number of possible crossover locations for each parent based on a minimum of six nucleotide overlap between parents. The differences in the total number of crossovers correlates with the sequence identity shared between parents. For example, parent 1 shares the most sequence identity with parents 2,3, and 4 and parent 4 shares the least sequence identity with parents 1, 2, and 3. FIG. 27B shows the number of crossover points that are consistent with generating a low schema disruption (<30, values from FIG. 25D). Even though the total number of crossover points is greater for parent 3, parent 4 has more potential crossover locations that are consistent with preserving the schema disruption. This provides an explanation and possible mechanism for the experimentally-observed absence of parent 3 in the improved chimeras previously reported by Crameri et al., 1998. Thus, calculations and comparisons of this kind can be used to predict optimal sets of parents for crossover recombination. In this calculation example, parent 3 (Yersinia enterocolitica) would not be used, because it contributes a relatively high crossover disruption in the schema disruption profile, in favor of the other parents, which exhibit less crossover disruption.

6.2 Crossover Recombination of β-Lactamase-like Genes by DNA Shuffling

This example describes experiments wherein the methods of the invention were used to evaluate a crossover probability distribution for a family shuffling experiment wherein four different β-lactamase-like genes (also referred to as cephalosporinase genes) were recombined. (See, Crameri et al. Nature, 391:288 (1998).

The three-dimensional structure for the backbone and side chain of the cephalosporinase protein expressed by Enterbacter cloacae was retrieved from that protein's high resolution crystal structure. Lobkovsky et al., Proc. Natl. Acad. Sci. USA., 90:11257-11261 (1993). Additional sequence information for the protein was retrieved from the TrEMBL database. (Bairoch & Apweiler, Nucl. Acids Res., 28:45-48 (2000) (Accession No. P05364). Sequences for homologous proteins expressed by other organisms were also retrieved from the SWISPROT database (Bairoch & Apweiler, supra), including sequences for cephalosporinase proteins expressed by Citrobacter freundii (Accession No. P05193), Klebsiella pneumonia (Accession No. P048437) and Yershinia entercolitica (Accession No. P45460).

Alignment of Parental Sequences.

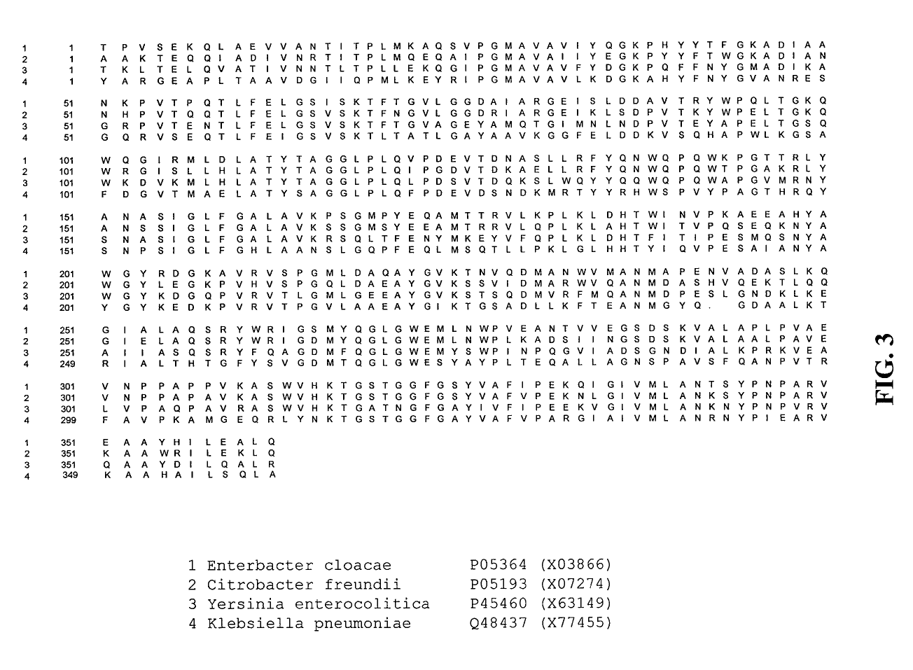

FIG. 3 is a gene alignment, using GAP, for four β-lactamase-like genes: (1) Enterobacter cloacae, (2) Citrobacter freundii, (3) Yersinia enterocolitica and (4) Klebsiella pneumonia. SWISPROT or TrEMBL accession numbers for the protein sequences and GenBank accession numbers for the DNA sequences are given. DNA sequences were retrieved from the GenBank database (Accession Nos. X03966, X07274, X63149 and X77455, respectively). These nucleotide sequences were also aligned, using the polypeptide sequence alignment shown in FIG. 3 to align codons of the DNA sequences that encoded aligned amino acid residues.

Generation of Crossover Mutants.

A library of possible recombinant mutants was generated in silico from the protein alignments using all possible “crossover locations” or “cut points” determined for the nucleic acid and protein alignments. Specifically, regions of four sequential amino acids in a first aligned sequence that were identical at the same positions in another aligned DNA sequence were identified as candidate crossover regions for the affected parents.

In this example, the parameter of four amino acids relates to a minimum required DNA identity shared between parents for DNA hybridization to occur. On the DNA level, six nucleotides of shared identity are required for hybridization to occur. The practical reason that the DNA limit (6) is lower than the amino acid limit (4×3=12 nucleotides) is because multiple codons can encode a single amino acid. This requires that a higher threshold be used when calculating the possible crossover points based on an amino acid alignment. Another approach would be to calculate the thermodynamic energy of hybridization based on the specific base pairs on each parent. See Moore et al., Predicting crossover generation in DNA shuffling, PNAS 98:6, 3226-3231 (2001). Also, melting temperature for the denaturation of the DNA overlap can be calculated based on the G-C, and A-T content. In this example, alignments are used to determine where sequences can reanneal. Alignments are not necessary for the calculations of Examples 6.1 and 6.3.

Two exemplary in silico methods were used to generate candidate hybrids or crossover mutants, based on the set of possible cut points determined from the alignment algorithm. In both methods, parental fragments are cut at the crossover locations which satisfy a predetermined crossover probability and are randomly recombined at those crossover points to produce a pool of recombined or hybrid proteins.

Method 1 (Random Probability Model of Fragment Extension).

To generate a candidate crossover mutant, a parent sequence was selected at random from the four cephalosporinase sequences. This sequence was written to the candidate mutant sequence up to a possible cut point. Upon reaching the possible cut point, a random number between 0 and 1 was chosen, and if the number was below a predetermined crossover probability P_{c}, then a second parent was randomly chosen. (Note that because each parent template is randomly selected for extension at a crossover point, the second parent could in some cases be the same as the first parent.) The mutant sequence was then extended from the cut point using the sequence of the second parent as a template, up until a next cut point was reached. Then, a random number between 0 and 1 was again chosen. If this number was below a predetermined crossover probability P_{c}, then the mutant sequence was extended from the cut point using another randomly selected parent as a template, up to the next cut point. In each case that the random number was not below a predetermined crossover probability P_{c}, the mutant sequence was extended to the next cut point by continuing with the same parent, i.e. without crossing over to another parent sequence. The probability PC can be the same or different for each cut point. These steps were repeated until the sequence was complete, e.g. a full-length hybrid protein was generated, comprising fragments of different parents recombined at selected cut points.

This process was repeated many times, each time with a randomly selected parent, until between about 10⁴ to 10⁶ full length cephalosporinase crossover mutants were generated.

The crossover probability using Method 1 was based roughly on fragment size and in this example was selected to P_{c}=0.30. In addition, a further instruction was imposed where each polypeptide fragment must be at least eight amino acid residues in length before another crossover was allowed to occur. The minimum fragment size of eight amino acids reflects a lower experimental bound relevant to the Stemmer protocol when the beta-lactamase genes were shuffled. In the DNA shuffling protocol, very small DNA fragments get “lost” in the reaction mixture and cannot become part of a recombinant mutant. Thus, this parameter is only relevant for Stemmer-like shuffling experiments and is not important for other methods (e.g., StEP has no minimum fragment size). This rule is not connected with disruption theory. Using these parameters, the average number of fragments per recombinant mutant was 13.4, corresponding to an average of 80-100 nucleotides per fragment. This was set to model results that were previously reported in actual directed evolution experiments. See, FIG. 1B, FIG. 12 and Crameri et al., Nature, 391:288 (1998).

Method 2 (Random Probability Model to Generate and Anneal Parent Fragments)

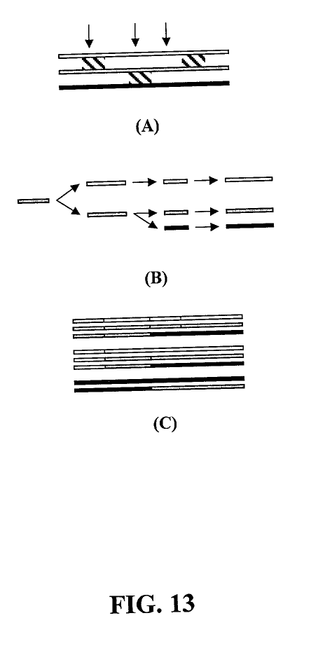

An alternative method, Method 2, was also used to generate candidate crossover mutants by DNA shuffling. This method is represented diagrammatically by FIG. 13. As shown by the arrows in FIG. 13A, parental strands are fragmented by randomly distributing cut points with probability P_{c}. In the figure, the arrows mark cut points and the thatched lines represent regions of sequence similarity between parents. In FIG. 13B, a parent is chosen at random to determine the first parental fragment. The next fragment is chosen amongst the parents that share adequate sequence identity (including the parent of the previous fragment) with equal probability. If the cut point at the end of the parent fragment corresponds to an identified crossover location based upon sequence identity, as described above, the next fragment is chosen from the pool of eligible parents, including the parent of the previous parent. This process is repeated until an entire offspring is created. The complete library of recombinant mutants that can be generated by the cut pattern shown. FIG. 13C. When this method was utilized to generate crossover mutants, the crossover probability in this example, based on fragment size, was set at P_{c}=0.15. As in Method 1, a further restriction was imposed so that each fragment must be at least eight amino acids in length. See also, FIG. 1A.

In this experiment, the number of fragments per mutant was 7, and the average fragment size was 80-100 nucleotides per fragment. As in Method 1, this is approximately the same number that has been previously reported. See, Crameri et al., Nature, 391:288 (1998).

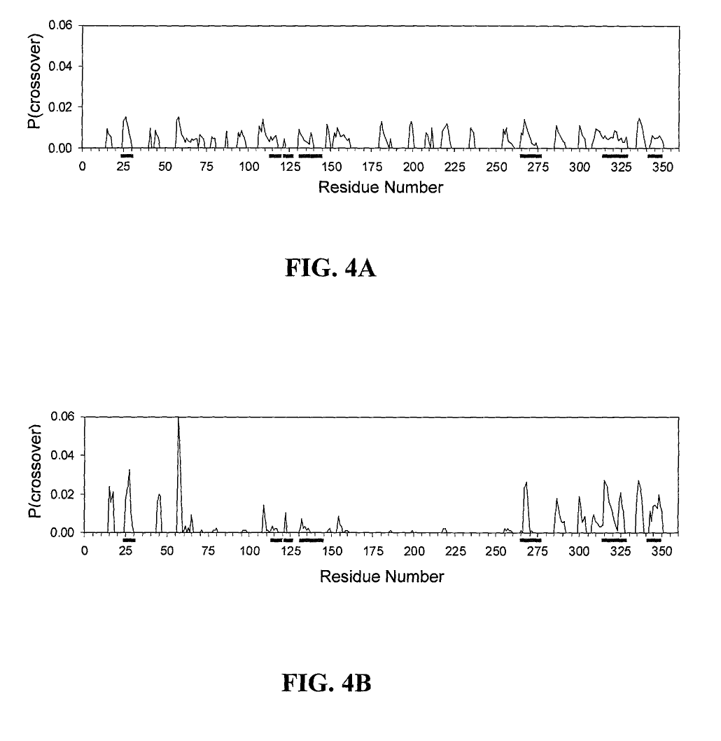

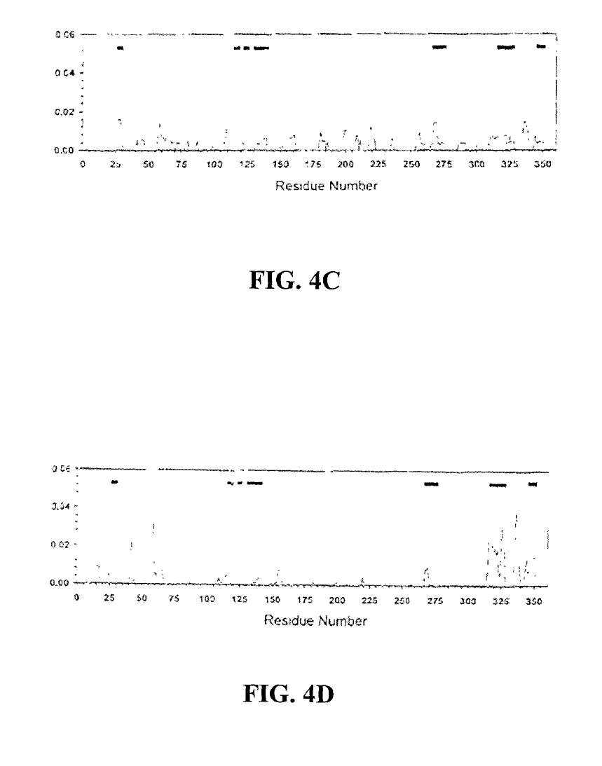

The distribution of crossover locations in the resulting library of crossover mutants is shown in FIGS. 4A and 4B using Method 1, and FIGS. 4C and 4D using Method 2. The graphs provided in these figures indicate the probability, P_{c}, that a mutant randomly selected from the library has a crossover point at a given amino acid residue (horizontal axis). The solid bars beneath the horizontal axis in this graph indicate residues where crossover points occurred in actual, functional mutants previously identified. Crameri et al. (1998).

In this exemplary model, the crossover probability P_{c} is related to fragment size and is the same at every residue. A residue is picked at random and a random number is chosen between 0 and 1. If this number is less than P_{c} then a crossover is “marked” on the sequence. This is repeated N times, where N is the number of residues. This effectively fragments the parent sequences for modeling purposes. The cut points that do not correspond to regions of adequate sequence identity are thrown out (FIG. 13) and the remaining crossovers are used to create all the possible recombinant mutants. A probability distribution for crossovers along the gene can be calculated by taking all the recombinant mutants generated by the algorithm and keeping track of where the crossovers occur. The number of times a crossover is observed is normalized by the total number of chimeric mutants generated to obtain a probability in silico.

Calculating Coupling Interactions.

Using the high resolution crystal structure obtained for the cephasporinase protein expressed by Enterobacter cloacae (Lobkovsky, supra.), coupling interactions were identified for all pairwise combinations of amino acid side chains. Specifically, the coupling interactions were delineated for each pair of amino acid residues, i and j, as a stability parameter e(i,j), with stability corresponding to the calculated energies of hydrogen bonds, electrostatic interactions and van der Waals interactions between side chains. To calculate such pairwise interactions, the DREIDING force field (Mayo et al., J. Phys. Chem., 94:8897 (1990)) was used in a modified form that included a hydrogen bonding parameter as previously described (Dahiyat et al., Science , 6:1333 (1997)). Pair-wise contributions of residues exhibiting interaction energies whose absolute value is greater than 0.25 kcal/mol were considered coupled for these examples.

Pair-wise interactions were also calculated using ORBIT protein design software that included parameters for hydrogen bonds, electrostatic interactions and van der Waals interactions between side chains.

Determining Crossover Disruption of Mutants.

Having thus identified pair-wise interactions between residues of the parent Enterobacter cloacae cephalosporinase proteins, each of the recombination mutants generated in silico was examined to identify coupling interactions that were originally present in the parent sequence(s), but had been disrupted in the crossover mutant. This demonstrates that Methods 1 and 2 generate random pools of mutants in silico that model the random pools generated in actual shuffling experiments. According to the invention, rules based on coupling interactions are used, as described, to eliminate many of the randomly generated candidates from consideration; i.e. the model focuses on optimum candidates which are more likely to exhibit desirable properties.

The average crossover disruption for the pool of crossover mutants shown in FIG. 4A was E_{c}407. The average crossover disruption for the pool of crossover mutants shown in FIG. 4C was E_{c}=44.

Screening Mutant Libraries.

The in silico crossover mutants were separated into two logical “bins”. The first bin included all crossover mutants generated. The second bin contained those mutants that exhibited a lower level of crossover disruption. In particular, the crossover mutants in the second bin had a crossover disruption level that fell below a preselected threshold (Eₜₕᵣₑₛₕ). The subpools of FIGS. 4B and 4D represent those chimeras that have crossover disruptions below the respective thresholds. The average crossover disruption is compared with a threshold. For instance, the threshold of 75 (FIG. 4B) was applied to the larger pool where the average disruption was 407 (FIG. 4A). The disruption threshold of 18 (FIG. 4D) was taken from the larger pool where the average disruption was 44 (FIG. 4C). In the first case, the smaller pool represents 1% of the larger pool and in the second case, the smaller pool represents 7.5% of the larger pool. The differences in the average disruption of the two large pools (407 versus 44) reflect several differences in the algorithm that was used to generate them. The difference is primarily due to the fact that amino acids that are identical in all of the parents were scored as disruptive when generating the crossover disruption of the chimeric mutants in FIGS. 4A and 4B (Pᵢ=Pⱼ=1.0 in Equation 2).

The distribution of crossover locations that produces minimum amounts of crossover disruption is shown in FIGS. 4B and 4D. The calculated probability P_{c} of a crossover event occurred at a nucleotide corresponding to each amino acid residue of the protein. The crossover probability is determined by counting the number of times a cut point occurs as a certain residue in a pool. The pool is generated by sequence identity alone or the pool the combines sequence identity with disruption. The number of times a cut point is observed is divided by the total number of chimeras in the pool. For example, if P_{c} (or P_{(croSS)}) of residue 25 is 0.02, this means that 2% of all chimeric mutants had a crossovers at residue 25. While the unscreened pool of mutants has an even distribution of crossover points in areas of sequence identity between the parental strands (FIGS. 4A and 4C), the screened pool has an uneven distribution of crossover points that are concentrated at the sequence termini.

Comparison with Previous Experiments.

FIGS. 4A and 4C show the probability distribution for cut points in β-lactamase calculated using DNA shuffling methods based upon sequence similarity. The variance in the maximum probability at each crossover is caused by the number of parents that share sequence identity at that point. The grey bars beneath the horizontal axis indicate actual crossovers that were observed in prior experiments (Crameri et al., Nature, 391;288 (1998)). The width of these bars is due to the inability to resolve the cut point to a single residue, due to the sequence similarity between parents. FIGS. 4B and 4D show the probability distribution when the additional constraint of low crossover disruption is imposed (Eₜₕᵣₑₛₕ).

As can be readily determined from FIGS. 4A and 4C, the unscreened pools of mutant hybrids show even distribution of crossover points in areas of sequence identity between the parental strands. However, as can be determined from FIGS. 4B and 4D, once the total pool is screened for mutants with minimal crossover disruption, the areas of parental sequence homology do not exhibit an even distribution of crossover points. For the proteins in these examples, computationally determined favorable crossover locations are found mainly at the termini of the sequences. These findings paralleled empirical observations (e.g. Crameri, supra.) The few crossover locations not located at the termini of the sequence correlated well with data from experiments by Stemmer. See, Stemmer, Proc. Natl. Acad. Sci. U.S.A., 91:10747 (1994); and Stemmer, Nature, 370:389 (1994).

The crossover probability distribution for the family shuffling experiment, in silico, starting with Citrobacter freundi, Klebseilla pneumoniae, Enterobacter cloacae, and Yersinia enterocolitica also corresponded well to previous in vivo experimental data where Yersinia enterocolitica was not observed in a pool of mutant offspring. Crameri, supra.

For example, FIG. 6 shows the crossover probability distribution for the family shuffling experiment with and without Yersinia enterocolitica as a starting parent. The dashed gray line represents the probability distribution for combinations of Citrobacter freundii, Klebseilla pneumoniae, Enterobacter cloacae. The solid black line is for the mutants containing the Yersinia enterocolitica sequence. The inclusion of Yersinia enterocolitica to the set of starting parents leads to the creation of a pool of recombinant mutant offspring with an increased probability of greater disruption of coupling interactions than the pool of recombinant mutant offspring without Yersinia enterocolitica. The generation of crossover disruption profiles, also called schema profiles, provides a mechanism by which the optimal parents can be determined.

Determination of Optimal Parents Based on an In Silico Chimera Library

Given the explosive growth of the gene databases due to the exhaustive sequencing of large numbers of organisms, sequences of homologous genes are easily accessible. Currently, the choice of starting parents for family shuffling is arbitrary or is made with minimal information (e.g., availability, sequence similarity). To date, there is no rigorous method to quantitatively use the information in the sequence databases to identify optimal starting parents. For example, in the beta-lactamase experiment discussed above, Stemmer and co-workers shuffled four genes, but only three of these genes were found in the improved recombinants (Crameri et al., 1998). The Yersinia enterocolitica gene (parent 3—FIG. 27) was not observed in the top mutants.

To understand this effect, the methods of the invention were applied to calculate all of the possible recombination locations between parents (1) Enterobacter cloacae, (2) Citrobacter freundii, (3) Yersinia enterocolitica, and (4) Klebsiella pneumoniae. FIG. 27. A potential crossover was recorded for every region shared between two parents that had six nucleotides in common. For instance, the total number of potential crossovers for parent 1 is the sum of the number of potential crossovers for parents 1-2, 1-3, and 1-4. The differences in the total number of crossovers for each parent reflects the sequence identity shared between parents. Because parent 3 shares more sequence identity with parents 1 and 2, than parent 4 does with parents 1 and 2, the total number of potential crossovers is greater. However, when the additional constraint of having a low schema disruption is imposed, parent 4 has more potential crossovers than parent 3. This provides a mechanism by which parent 4 was observed in the improved chimeras, but not parent 3.

6.3 Single Cut Point Recombination

The invention can also be applied to sets of parent biopolymers that do not share sequence identity, and using recombination methods that do not rely on sequence identity. At least two methods are known in the art for producing recombinant gene libraries having cross-overs at any position, regardless of sequence identity. See, e.g., Ostermier et al., Nature Biotechnology, 17:1205-1209 (1999); Ostermeier et al., Bioorg. Med. Chem., 7:2139-2144(1999); Sieber et al., Nature Biotechnology, 19,456-460(2000). In particular, these methods allow genes (and their corresponding polypeptides) that have diverged nucleotide sequences to be recombined. However, in the experimental implementation described here only two parent sequences are recombined with only a single cut point (crossover).

A method of the invention was used to simulate the recombination of PurN and GART glycinamide ribonucleotide transformylase (Ostermier et al., Nature Biotechnology, 17:1205-1209 (1999)). A coupling matrix was calculated using the three-dimensional structure of PurN previously described by Almassy et al. (Proc. Natl. Acad. Sci. USA., 89:6114 (1992)). The crossover disruption was then calculated for each possible single crossover mutant. FIG. 5 provides a plot showing the crossover disruption calculated for each mutant, indicated by the amino acid residue of the crossover location.

The range of amino acid sequences shown in FIG. 5 (i.e., amino acid residues 50-150 of the aligned glycinamid ribonucleotide transformylase proteins) correspond to crossover regions where non-homologous recombinations were previously constructed by Benkovic et al. Nature Biotechnology, 17:1205-1209 (1999). Crossover locations for functional crossover mutants that were identified in these previous experiments are indicated on the graph in FIG. 5 by horizontal lines. The vertical lines show the positions where single crossovers occurred and led to functional enzymes. The “2” indicates that this crossover was sampled twice in the library. The diamonds show where homologous recombination (DNA shuffling with single cut points) experiments produced crossovers. The calculated crossover disruption decreases rapidly outside of 50-150 amino acid sequence region indicating that, as expected, crossovers would be strongly biased towards the—and C-termini of the parents. Local crossover disruption minima are also present in the region between amino acid residues 50-150 shown in FIG. 5. These minima reflect the fact that glycinamid ribonucleotide transformylase proteins comprise at least two topologically separate domains. Thus, the local minima in crossover disruption reflect crossover points which occur at the intersection of such separate domains.

It will be appreciated by persons of ordinary skill in the art that the examples herein are illustrative only, and do not limit the scope of the invention or the accompanying claims.

We claim:

1. A method for selecting a crossover location in a first biopolymer having a first polymer sequence, for recombination with one or more second biopolymers each having its own second polymer sequence, which method comprises:
identifying coupling interactions between pairs of residues in the first polymer sequence;
generating a plurality of data structures, each data structure representing a crossover mutant comprising a recombination of the first and a second polymer sequence wherein each recombination has a different crossover location;
determining, for each data structure, a crossover disruption related to the number of coupling interactions disrupted in the crossover mutant represented by the data structure; and
identifying, among the plurality of data structures, a particular data structure having a crossover disruption below a threshold,
wherein the crossover location of the crossover mutant represented by the particular data structure is the identified crossover location.

2. A method of claim 1, wherein the particular polymer sequence comprises a sequence of amino acid residues.

3. A method of claim 1, wherein the particular polymer sequence comprises a sequence of nucleotide residues.

4. A method of claim 1, wherein coupling interactions are identified by use of a coupling matrix.

5. A method of claim 1, wherein the coupling matrix is the summation of all the coupling interactions of the first polymer sequence.

6. A method of claim 1, wherein coupling interactions are identified by a determination of a conformational energy between residues.

7. A method of claim 1, wherein coupling interactions are identified by a determination of interatomic distances between residues.

8. A method of claim 6, wherein conformational energies for each of the first and second polymer sequences are determined from a three-dimensional structure for at least one of the first and second polymer sequences.

9. A method of claim 7, wherein interatomic distances for each of the first and second polymer sequences are determined from a three-dimensional structure for at least one of the first and second polymer sequences.

10. A method of claim 2, wherein coupling interactions are identified by a conformational energy between residues above a threshold.

11. A method of claim 1, wherein a coupling interaction between a pair of residues in the first polymer sequence is disrupted in a crossover mutant wherein a coupling interaction between a pair of residues is disrupted in a crossover mutant if the identity of both residues participating in the coupling interaction is different than that which exists in any of the parents.

12. A method of claim 8, wherein a coupling interaction between a pair of residues in the first polymer sequence is disrupted in a crossover mutant wherein a coupling interaction between a pair of residues is disrupted in a crossover mutant if the identity of both residues participating in the coupling interaction is different than that which exists in any of the parents.

13. A method of claim 1, wherein the crossover disruption is the summation of all coupled interactions in the parent that are considered disrupted in the data structure representing the crossover mutant.

14. A method of claim 1, wherein the threshold is an average level of crossover disruption for the plurality of data structures.

15. A method of claim 1, wherein the threshold is at least one standard deviation below the average level for the plurality of data structures.

16. A method of claim 1, wherein the threshold is set so that approximately 7.5% of the total number of generated data structures is below the threshold.

17. A method of claim 1, wherein the threshold is set so that approximately 1% of the total number of generated data structures is below the threshold.

18. A method of claim 1, wherein the threshold is set so that approximately 0.001 % of the total number of generated data structures is below the threshold.

19. A method of claim 1, wherein the generation of crossover mutants comprises:
the sequence alignment of a plurality of biopolymers;
the identification of possible cut points in the biopolymer based upon regions of sequence identity identified by the sequence alignment; and
the generation of single crossover mutants based upon the identified possible cut points.

20. A method of claim 19, wherein the regions of sequence identity must contain at least 4 residues.

21. A method of claim 19, there must be at least eight residues between crossovers.

22. A method of claim 1, wherein the generation of the plurality of data structures comprises:
the sequence alignment of a plurality of biopolymers using simulated annealing with non-homologous parents;
selecting crossover locations based upon the minimization of crossover disruption, fragment size, starting number of parents; and
the generation of a plurality of data structures based upon the identified possible crossover locations.

23. A method of claim 1, wherein the generation of the plurality of data structures comprises:
choosing one of the biopolymers from the plurality of biopolymers at random;
copying the biopolymer until a possible crossover location is reached;
choosing a random number between 0 and 1;
choosing a new biopolymer from the plurality of biopolymers to copy to the offspring if the random number is below a crossover probability (P_{c}); and
repeating the above process until the data structure representing the crossover mutant is the desired length.

24. A method of claim 19, wherein the generation of the plurality of data structures based upon identified cut points comprises:
cutting the biopolymers in into biopolymer fragments by randomly assigning cut points with a set probability;
randomly choosing one of the biopolymer fragments as a starting parent;
randomly identifying another biopolymer fragment from the total pool of the biopolymer fragments;
ligating the identified biopolymer fragment to the parent fragment, if the identified fragment has a sequence identity cut-point at the end of the fragment; and
repeating the randomly identifying step until the data structure, representing the crossover mutant is the desired length.

25. A method for directed evolution of a polymer, which method comprises steps of:
providing a plurality of parent polymer sequences;
identifying crossover locations in the parent polymer sequences for recombination according to claim 1;
generating one or more mutant polymer sequences utilizing recombinatory techniques targeted at the identified crossover locations on the parent polymer sequences;
screening the one or more mutant sequences for the one or more properties of interest; and
selecting at least one mutant sequence where one or more properties of interest are identified.

26. A method according to claim 25, wherein the method is iteratively repeated, and wherein at least one mutant sequence selected in a first iteration is a parent sequence in a second iteration.

27. A method of claim 25, wherein the recombination techniques are selected from the group consisting of: DNA shuffling, StEP method, fragmentation and reassembly, synthesis, and random-priming recombination.

28. A computer system for analyzing a polymer sequence, which computer system comprises:
memory and a processor interconnected with the memory and having one or more software components loaded therein, wherein the one or more software components cause the processor to execute steps of a method according to claim 1.

29. A computer system of claim 28, wherein the software components comprise a database of polymer sequences.

30. A computer system of claim 28, wherein the software components comprise a database of three-dimensional structures for polymer sequences.

31. A computer program comprising a computer readable medium having one or more software components encoded in computer readable form, wherein the one or more software components may be loaded into a memory of a computer system and cause a processor interconnected with the memory to execute steps of a method according to claim 1.

32. A computer program according to claim 30, wherein the computer readable medium further has, encoded thereon in computer readable form, a database of polymer sequences.

33. A computer program according to claim 30, wherein the computer readable medium further has, encoded thereon in computer readable form, a database of three-dimensional structures for polymer sequences.

34. A computer system for analyzing a polymer sequence, which computer system comprises:
memory and a processor interconnected with the memory and having one or more software components loaded therein, wherein the one or more software components cause the processor to execute steps of a method according to claim 19.

35. A computer program comprising a computer readable medium having one or more software components encoded in computer readable form, wherein the one or more software components may be loaded into a memory of a computer system and cause a processor interconnected with the memory to execute steps of a method according to claim 19.

36. A computer system for analyzing a polymer sequence, which computer system comprises:
memory and a processor interconnected with the memory and having one or more software components loaded therein, wherein the one or more software components cause the processor to execute steps of a method according to claim 23.

37. A computer program comprising a computer readable medium having one or more software components encoded in computer readable form, wherein the one or more software components may be loaded into a memory of a computer system and cause a processor interconnected with the memory to execute steps of a method according to claim 23.

38. A computer system for analyzing a polymer sequence, which computer system comprises:
memory and a processor interconnected with the memory and having one or more software components loaded therein, wherein the one or more software components cause the processor to execute steps of a method according to claim 24.

39. A computer program comprising a computer readable medium having one or more software components encoded in computer readable form, wherein the one or more software components may be loaded into a memory of a computer system and cause a processor interconnected with the memory to execute steps of a method according to claim 24.

40. A computer system for analyzing a polymer sequence, which computer system comprises:
memory and a processor interconnected with the memory and having one or more software components loaded therein, wherein the one or more software components cause the processor to execute steps of a method according to claim 25.

41. A method for producing hybrid polymers from two or more parent polymers comprising the steps of:
identifying structural domains of at least one parent polymer;
organizing identified domains into schema;
calculating a schema disruption profile;
selecting at least one crossover location based on the schema disruption profile; and
recombining two or more parent polymers at one or more selected crossover locations to produce at least one hybrid polymer.

42. A method of claim 41, wherein parent polymers are recombined in silico, in vitro, in vivo, or in any combination thereof.

43. A method of claim 41, wherein parent polymers are recombined in silico to produce at least one candidate hybrid polymer.

44. A method of claim 43, wherein parent polymers are physically recombined at one or more crossover locations, including at least one selected crossover location, to produce at least one hybrid polymer corresponding to a candidate hybrid polymer.

45. A method of claim 44, wherein parent polymers are physically recombined in vitro.

46. A method of claim 44, wherein parent polymers are physically recombined in vivo.

47. A method of claim 41, wherein each parent polymer comprises a polypeptide.

48. A method of claim 44, wherein each parent polymer comprises a polypeptide.

49. A method of claim 4 1, wherein each parent polymer comprises an oligonucleotide.

50. A method of claim 44, wherein each parent polymer comprises an oligonucleotide.

51. A method of claim 44, wherein the parent polymers are one of polypeptides and oligonucleotides, and wherein parent polymers are recombined in a directed evolution experiment.

52. A method of claim 44, comprising the step of screening hybrid polymers for one or more properties.

53. A method of claim 51, comprising the step of screening hybrid polymers for one or more properties.

54. A method of claim 5 1, wherein the directed evolution experiment includes at least one protocol selected from the group consisting of fragmentation and reassembly, family shuffling, exon shuffling, StEP, ITCHY, synthesis techniques, and PCR-based techniques.

55. A method of claim 44, wherein hybrid polymers are expressed by host cells.

56. A method of claim 41, wherein hybrid polymers are expressed by host cells.

57. A method of claim 53, wherein hybrid polymers are expressed by host cells.

58. A method of claim 41, wherein crossover locations are selected from a schema disruption profile based on a prediction that the selected crossovers will tend to produce relatively less schema disruption than other crossover locations.

59. A method of claim 44, wherein crossover locations are selected from a schema disruption profile based on a prediction that the selected crossovers will tend to produce relatively less schema disruption than other crossover locations.

60. A method of claim 41, wherein crossover locations are selected based on a schema disruption threshold.

61. A method of claim 44, wherein crossover locations are selected based on a schema disruption threshold.

62. A method of claim 51, wherein crossover locations are selected based on a schema disruption threshold.

63. A method of claim 41, wherein crossover locations are selected to preserve schema from at least one parent polymer.

64. A method of claim 41, wherein crossover locations are selected to preserve schema from a plurality of parent polymers.

65. A method of claim 44, wherein crossover locations are selected to preserve schema from at least one parent polymer.

66. A method of claim 44, wherein crossover locations are selected to preserve schema from a plurality of parent polymers.

67. A method of claim 51, wherein crossover locations are selected to preserve schema from at least one parent polymer.

68. A method of claim 51, wherein crossover locations are selected to preserve schema from a plurality of parent polymers.

69. A method of claim 44, wherein a library of candidate hybrid polymers is compared with a library of physically recombined hybrid polymers.

70. A method of claim 51, wherein the sequence space of a directed evolution experiment is reduced based on a library of in silico candidate hybrid candidate sequences.

71. A method for producing a library of hybrid polymers comprising the steps of:
choosing two or more parent polymers;
identifying structural domains of at least one parent polymer;
organizing identified domains into schema;
calculating a schema disruption profile;
selecting crossover locations based on the schema disruption profile;
recombining two or more parent polymers at one or more selected crossover locations to produce a set of hybrid polymers;
repeating at least the choosing and recombining steps to produce at least one additional set of hybrid polymers; and
generating a library of hybrid polymers from the sets of hybrid polymers.

72. A method of claim 71, wherein the repeated steps comprise choosing at least one hybrid polymer as a parent polymer.

73. A method of claim 71, wherein recombining steps are performed in silico.

74. A method of claim 73, further comprising physically recombining parent polymers at selected crossover locations to produce hybrids in the library.

75. A method of claim 71, wherein schema are common to at least two parents.

76. A method of claim 74, wherein schema are common to at least two parents.

77. A method of claim 71, wherein a schema disruption profile is calculated based on one or both of conformational energy and interatomic distances.

78. A method of claim 75, wherein a schema disruption profile is calculated based on one or both of conformational energy and interatomic distances.

79. A method of claim 73, wherein parent polymers are physically recombined in a directed evolution experiment.

80. A method of claim 79, wherein the directed evolution experiment includes at least one protocol selected from the group consisting of fragmentation and reassembly, family shuffling, exon shuffling, StEP, ITCHY, synthesis techniques, and PCR-based techniques.

81. A method of claim 74, further comprising screening hybrids in the library for one or more properties.

82. A method of claim 73, further comprising physically recombining parent polymers at selected crossover locations to produce hybrids in the library and screening hybrids in the library for one or more properties; and wherein the repeated steps comprise choosing at least one hybrid polymer as a parent polymer based on screening results.

83. A method of claim 41, wherein schema comprise domains identified according to sequence alignments between two or more parent polymers.

84. A method of claim 71, wherein schema comprise domains identified according to sequence alignments between two or more parent polymers.

85. A method of claim 41, wherein the crossover location comprises a crossover region.

86. A method of claim 71, wherein the crossover location comprises a crossover region.

87. A method of claim 41, wherein the schema disruption profile comprises fitness contributions of polymer residues of one or more parent polymers.

88. A method of claim 71, wherein the schema disruption profile comprises fitness contributions of polymer residues of one or more parent polymers.

89. A method of claim 41, further comprising the step of calculating a crossover disruption profile.

90. A method of claim 71, further comprising the step of calculating a crossover disruption profile.

91. A method of claim 41, further comprising restricting the selection of crossover locations based on at least one predetermined constraint.

92. A method of claim 91, wherein the predetermined constraint is based on a protocol for physically recombining the polymers.

93. A method of claim 92, wherein the predetermined constraint comprises at least one of a requirement of sequence identity between parents, a constraint on the number of crossovers, and a constraint on the location of crossovers.

94. A method of claim 41, further comprising the steps of generating a coupling matrix and using the matrix in at least one of the identifying, organizing, calculating, and selecting steps.

95. A method of claim 71, further comprising the steps of generating a coupling matrix and using the matrix in at least one of the identifying, organizing, calculating, and selecting steps.

96. A method of claim 41, wherein domains are identified based on sequence information for at least one parent polymer.

97. A method of claim 71, wherein domains are identified based on sequence information for at least one parent polymer.

98. A method of claim 41, wherein domains are identified based on a crystal structure for at least one parent polymer.

99. A method of claim 71, wherein domains are identified based on a crystal structure for at least one parent polymer.

100. A method of claim 71, wherein crossover locations are selected from a schema disruption profile based on a threshold disruption value.

101. A method for modeling the recombination of two or more parent polymers comprising the steps of:
obtaining structural information for at least one parent polymer;
evaluating coupling interactions between polymer residues based on the structural information;
identifying domains based on the determined coupling interactions;
calculating the crossover disruption of the identified domains to produce a disruption profile;
applying a predetermined threshold disruption to each domain of the disruption profile;
at least one of, accepting domains which satisfy the threshold and rejecting domains which do not satisfy the threshold;
repeating at least the identifying, calculating and applying steps until each identified domain is accepted or rejected;
designating the accepted or rejected domains as disruptive;
selecting crossover regions from domains that are not designated as disruptive; and
recombining parent polymers at selected crossover regions.

102. A method of claim 101, wherein the step of identifying domains comprises determining the polymer residues which belong to each domain, and the step of selecting crossover regions comprises specifying one or more residues within at least one non-disruptive domain.

103. A method of claim 101, wherein the threshold disruption represents a maximum allowable disruption, domains having a disruption above the threshold are accepted as disruptive and are preserved, domains having a disruption below the threshold are rejected as non-disruptive and may be altered, and crossover regions are selected from residues belonging to non-disruptive domains.

104. A method of claim 103, wherein domains having a disruption equal to the threshold are one of accepted as disruptive or rejected as non-disruptive.

105. A method of claim 102, wherein the selection of crossover regions is restricted according to one or more recombination constraints.

106. A method of claim 104, wherein the selection of crossover regions is restricted according to one or more recombination constraints.

107. A method of claim 105, wherein the constraint comprises at least one of a requirement of sequence identity between parents, a constraint on the number of crossovers, and a constraint on the location of crossovers.

108. A method of claim 106, wherein the constraint comprises at least one of a requirement of sequence identity between parents, a constraint on the number of crossovers, and a constraint on the location of crossovers.

109. A method of claim 105, wherein the constraint comprises a requirement of sequence identity between parents, and the method further comprises:
obtaining sequence information for the parent polymers;
aligning the obtained sequence information; and
identifying cut points within aligned regions of the parent sequences.

110. A method of claim 109, where the step of identifying cut points comprises selecting cut points having a relatively low crossover disruption, and the step of specifying a set of parental fragments for recombination based on selected cut points.

111. A method of claim 44, wherein parental polymers are genes, and the polymers are physically recombined by a staggered extension process (StEP) comprising the steps of:
specifying one or more selected crossover locations;
cutting each of two or more parent polymers within one or more crossover regions that each encompass one or more specified crossover locations to define a set of polymer fragments;
producing a set of defined polymer fragments, wherein each fragment has an end primer comprising a sequence with residues that extend past a specified crossover location; and
assembling at least one of pair of fragments having sequences which overlap an end primer of at least one fragment of the pair, to produce a recombinant polymer.

112. A method of claim 111, wherein the producing step comprises synthesizing two or more fragments.

113. A method of claim 112, wherein synthesizing fragments comprises split pool synthesis.

114. A method of claim 111, wherein fragments are assembled by extension from an end primer.

115. A method of claim 111, wherein the set of defined polymer fragments comprises all of the fragments arising from cutting all of the parent polymers within all of the crossover regions that encompass all of the specified crossover locations.

116. A method of claim 115, wherein all of the fragments are assembled in all of the possible combinations.

117. A method of claim 111, further comprising the step of screening one or more recombinant polymers for a property.

118. A method of claim 74, wherein parental polymers are genes, and the polymers are physically recombined by a staggered extension process (StEP) comprising the steps of:
specifying one or more selected crossover locations;
cutting each of two or more parent polymers within one or more crossover regions that each encompass one or more specified crossover locations to define a set of polymer fragments;
producing a set of defined polymer fragments, wherein each fragment has an end primer comprising a sequence with residues that extend past a specified crossover location;
assembling at least one of pair of fragments having sequences which overlap an end primer of at least one fragment of the pair, to produce a recombinant polymer.

119. A method of claim 118, wherein fragments are assembled by extension from an end primer.

120. A method of claim 111, wherein the set of defined polymer fragments comprises all of the fragments arising from cutting all of the parent polymers within all of the crossover regions that encompass all of the specified crossover locations; and wherein all of the fragments are assembled in all of the possible combinations.

121. A method of claim 44, wherein the parental polymers are genes, and the polymers are physically recombined by an in vitro-in vivo recombination method comprising the steps of:
shuffling at least two parent polymers to produce a set of parental fragments having selected crossover locations;
assembling fragments at crossover locations by overlap extension and gap repair, to provide double stranded sequences containing mismatched regions; and
repairing the mismatched regions in vivo by inserting the double-stranded sequences into a host cell to provide a library of crossover recombinants.

122. A method of claim 121, wherein the double stranded sequences are inserted into a host cell in the form of a heteroduplex plasmid.

123. A method of claim 121, wherein parental homoduplexes are removed.

124. A method of claim 44, wherein the parental polymers are genes, and the polymers are physically recombined by an in vitro-in vivo recombination method comprising the steps of:
specifying one or more selected cut points;
preparing synthetic polymer fragments having sequences corresponding to the sequences of parent polymers that are cut at specified cut points;
extending the sequence of each fragment at a cut point against a parental template to produce a set of polymer duplexes representing different combinations of fragments;
removing parent homoduplex polymers; and
providing a set of recombinants from the resulting heteroduplex polymers.

125. A method of claim 124, wherein parent homoduplexes are removed by inserting the polymer duplexes into a host cell.

126. A method of claim 125, wherein the polymer duplexes are inserted into a host cell in the form of a heteroduplex plasmid.

127. A method of claim 74, wherein the parental polymers are genes, and the polymers are physically recombined by an in vitro-in vivo recombination method comprising the steps of:
specifying one or more selected cut points;
providing polymer fragments having sequences corresponding to the sequences of parent polymers that are cut at specified cut points;
extending the sequence of each fragment at a cut point against a parental template to produce a set of polymer duplexes representing different combinations of fragments;
removing parent homoduplex polymers; and
providing a set of recombinants from the resulting heteroduplex polymers.

128. A method of claim 127, wherein parent homoduplexes are removed by inserting the polymer duplexes into a host cell.

129. A method of claim 128, wherein the polymer duplexes are inserted into a host cell in the form of a heteroduplex plasmid.

130. A method of claim 127, further comprising the step of screening one or more recombinant polymers for a property.

131. A method of claim 44, wherein the parental polymers are genes, and the polymers are physically recombined by a PCR amplification method comprising the steps of:
specifying one or more selected cut points;
defining polymer fragments having sequences corresponding to the sequences of parent polymers that are cut at specified cut points;
providing sets of primers, wherein each primer in a set hybridizes to all parent strands at a crossover region corresponding to a specified cut point;
producing a set of defined fragments from each parent polymer by PCR amplification with each set of primers; and
assembling fragments in a pool by PCR amplification.

132. A method of claim 131, wherein:
each set of primers is a pair of terminal primers or a pair of intervening primers;
each primer in a terminal pair of primers corresponds to at least one terminal end of one parent polymer; and
each primer in each intervening pair of primers corresponds to a specified cut point.

133. A method of claim 132, wherein PCR amplification is performed using a first primer selected a first pair of primers, and a second primer selected from a second pair of primers.

134. A method of claim 133, wherein the first and second primers flank the ends of a polymer fragment.

135. A method of claim 74, wherein the parental polymers are genes, and the polymers are physically recombined by a PCR amplification method comprising the steps of:
specifying one or more selected cut points;
defining polymer fragments having sequences corresponding to the sequences of parent polymers that are cut at specified cut points;
providing sets of primers, wherein each primer in a set hybridizes to all parent strands at a crossover region corresponding to a specified cut point;
producing a set of defined fragments from each parent polymer by PCR amplification with each set of primers; and
assembling fragments in a pool by PCR amplification.

136. A method of claim 135, wherein:
each set of primers is a pair of terminal primers or a pair of intervening primers;
each primer in a terminal pair of primers corresponds to at least one terminal end of one parent polymer; and
each primer in each intervening pair of primers corresponds to a specified cut point.

137. A method of claim 136, wherein PCR amplification is performed using a first primer selected a first pair of primers, and a second primer selected from a second pair of primers.

138. A method of claim 137, wherein the first and second primers flank the ends of a polymer fragment.

139. A method of claim 131, further comprising the step of screening one or more recombinant polymers for a property.

140. A method of claim 44, wherein the parental polymers are genes, and the polymers are physically recombined by a family shuffling method comprising the steps of:
specifying one or more selected crossover locations;
providing sets of primer pairs, wherein each primer of each pair comprises sequences from two parent polymers which span and include a specified crossover location;
producing fragments of the parent polymers;
reassembling the fragments in the presence of the primers using PCR amplification.

141. A method of claim 74, wherein the parental polymers are genes, and the polymers are physically recombined by a family shuffling method comprising the steps of:
specifying one or more selected crossover locations;
providing sets of primer pairs, wherein each primer of each pair comprises sequences from two parent polymers which span and include a specified crossover location;
producing fragments of the parent polymers;
reassembling the fragments in the presence of the primers using PCR amplification.

142. A method of producing recombinant oligonucleotides from two or more parent oligonucleotides by a staggered extension process comprising the steps of:
selecting one or more crossover locations for each parent oligonucleotide;
cutting each of two or more parents within one or more crossover regions that each encompass one or more specified crossover locations to define a set of fragments;
producing a set of defined fragments, wherein each fragment has an end primer comprising a sequence with residues that extend past a specified crossover location; and
assembling at least one of pair of fragments having sequences which overlap an end primer of at least one fragment of the pair, to produce a recombinant oligonucleotide.

143. A method of producing recombinant oligonucleotides from two or more parent oligonucleotides by an in vitro-in vivo recombination method comprising the steps of:
selecting one or more crossover locations for each parent oligonucleotide;
shuffling at least two parent oligonucleotides to produce a set of fragments having selected crossover locations;
assembling fragments at crossover locations by overlap extension and gap repair, to provide double stranded sequences containing mismatched regions; and
repairing the mismatched regions in vivo by inserting the double-stranded sequences into a host cell to provide a library of crossover recombinants.

144. A method of producing recombinant oligonucleotides from two or more parent oligonucleotides by an in vitro-in vivo recombination method comprising the steps of:
specifying one or more selected cut points for each parent oligonucleotide;
preparing synthetic polymer fragments having sequences corresponding to the sequences of parent oligonucleotides that are cut at specified cut points;
extending the sequence of each fragment at a cut point against a parental template to produce a set of oligonucleotide duplexes representing different combinations of fragments;
removing parent homoduplex oligonucleotides; and
providing a set of recombinants from the resulting heteroduplex oligonucleotides.

145. A method of claim 144, wherein the oligonucleotide duplexes are removed by inserting oligonucleotide duplexes into a host cell in the form of a heteroduplex plasmid.

146. A method of producing recombinant oligonucleotides from two or more parent oligonucleotides by a PCR amplification method comprising the steps of:
specifying one or more selected cut points for each parent oligonucleotide;
defining oligonucleotide fragments having sequences corresponding to the sequences of parent oligonucleotides that are cut at specified cut points;
providing sets of primers, wherein each primer in a set hybridizes to all parent strands at a crossover region corresponding to a specified cut point;
producing a set of defined fragments from each parent by PCR amplification with each set of primers; and
assembling fragments in a pool by PCR amplification.

147. A method of claim 146, wherein:
each set of primers is a pair of terminal primers or a pair of intervening primers;
each primer in a terminal pair of primers corresponds to at least one terminal end of one parent polymer; and
each primer in each intervening pair of primers corresponds to a specified cut point.

148. A method of claim 147, wherein PCR amplification is performed using a first primer selected a first pair of primers, and a second primer selected from a second pair of primers.

149. A method of claim 148, wherein first and second primers flank the ends of a fragment.

150. A method of producing recombinant oligonucleotides from two or more parent oligonucleotides by a family shuffling method comprising the steps of:
specifying one or more selected crossover locations for each parent oligonucleotide;
providing sets of primer pairs, wherein each primer of each pair comprises sequences from two parents which span and include a specified crossover location;
producing fragments of the parent polymers;
reassembling the fragments in the presence of the primers using PCR amplification.

151. A method of claim 1, wherein a coupling interaction between a pair of residues in the first polymer sequence is disrupted in a crossover mutant if the identity of a residue is different in the crossover mutant than in the first polymer sequence, and wherein a coupling interaction between a pair of residues is scaled by the probabilities that the identity and sequence position of the coupled residues are the same in both parents.
